(19) United States
(12) Patent Application Publication
(10) Pub. No.: US 2018/0356419 A1
(43) Pub. Date: Dec. 13, 2018

(54) BIOMARKERS FOR DETECTION OF TUBERCULOSIS RISK

(71) Applicants: SOMALOGIC, INC., Boulder, CO (US); UNIVERSITY OF CAPE TOWN, Cape Town (ZA); SEATTLE BIOMEDICAL RESEARCH INSTITUTE D/B/A THE CENTER FOR INFECTIOUS DISEASE RESEARCH, Seattle, WA (US)

(72) Inventors: Thomas HRAHA, Boulder, CO (US); David STERLING, Boulder, CO (US); Urs A. OCHSNER, Boulder, CO (US); Nebojsa JANJIC, Boulder, CO (US); Thomas Jens SCRIBA, Cape Town (ZA); Adam Garth PENN-NICHOLSON, Cape Town (ZA); Willem Albert HANEKOM, Cape Town (ZA); Daniel Edward ZAK, Seattle, WA (US); Ethan Greene THOMPSON, Seattle, WA (US)

(73) Assignees: SOMALOGIC, INC., Boulder, CO (US); UNIVERSITY OF CAPE TOWN, Cape Town (ZA); SEATTLE BIOMEDICAL RESEARCH INSTITUTE D/B/A THE CENTER FOR INFECTIOUS DISEASE RESEARCH, Seattle, WA (US)

(21) Appl. No.: 15/572,480

(22) PCT Filed: May 7, 2016
(86) PCT No.: PCT/US2016/031383

§ 371 Date: Nov. 7, 2017

Related U.S. Application Data
(60) Provisional application No. 62/159,011, filed on May 8, 2015.

(51) Int. Cl. G01N 33/569

(52) U.S. Cl. CPC G01N 33/5695; G01N 2800/52; G01N 2800/26; G01N 2333/70503; G01N 2333/4716; G01N 2333/4745; G01N 2333/70596

(57) ABSTRACT

The present application relates generally to biomarkers for tuberculosis (TB) infection and disease and methods of detection thereof. In various embodiments, the invention relates to one or more biomarkers, biomarker panels, methods, devices, reagents, systems, and kits for detecting and/or characterizing TB infection and/or disease.

This application claims the benefit of priority of U.S. Provisional Application No. 62/159,011, filed May 8, 2015, which is incorporated by reference herein in its entirety for any purpose.

### FIELD

The present application relates generally to biomarkers for determining the risk of a subject with latent tuberculosis (TB) infection developing active TB disease, and methods of use thereof. In various embodiments, the invention relates to one or more biomarkers, biomarker panels, methods, devices, reagents, systems, and/or kits for detecting and/or characterizing the risk of a subject with a latent TB infection developing active TB disease.

### BACKGROUND

Tuberculosis (TB) is a disease caused by Mycobacterium tuberculosis and other disease causing mycobacteria. The bacteria usually attack the lungs, but TB bacteria can attack any part of the body such as the kidney, spine, and brain. If not treated properly, TB disease can be fatal. Not everyone infected with TB bacteria becomes sick. As a result, two TB-related conditions exist: latent TB infection and active TB disease. Both latent TB infection and active TB disease can be treated.

### SUMMARY

In some embodiments, methods of determining the risk of a subject with latent tuberculosis (TB) infection developing active TB disease are provided.

In some embodiments, a method comprises detecting the presence or level of at least one, at least two, at least three, at least four, at least five, at least six, at least seven, or eight biomarkers selected from the biomarkers in Table 11 in a sample (e.g., plasma, serum, urine, saliva, etc.) from the subject. In some embodiments, the subject is identified as having a latent TB infection that is likely to transition into active TB disease if the level of at least one, at least two, at least three, at least four, at least five, at least six, at least seven, or eight of the biomarkers is altered relative to a control level of the respective biomarker. In some embodiments, a method comprises detecting the levels of a set of biomarkers from Table A, Table B, or Table C in a sample (e.g., plasma, serum, urine, saliva, etc.) from the subject. In some embodiments, a method comprises detecting the levels of a set of biomarkers from Table C in a sample (e.g., plasma, serum, urine, saliva, etc.) from the subject. In some embodiments, a method comprises detecting the levels of a set of biomarkers from Table C in a sample (e.g., plasma, serum, urine, saliva, etc.) from the subject. In some embodiments, a method comprises detecting at least one, at least two, at least three, at least four, or five biomarkers selected from C9, IGFBP-2, CD79A, MXRA7, and NR-CAM in a sample (e.g., plasma, serum, urine, saliva, etc.) from the subject. In some embodiments, a method comprises detecting the levels of C9, IGFBP-2, CD79A, MXRA7, and NR-CAM in a sample (e.g., plasma, serum, urine, saliva, etc.) from the subject. In some embodiments, the subject is identified as having a latent TB infection that is likely to transition into active TB disease if the level of at least one, at least two, at least three, at least four, at least five, at least six, at least seven, at least eight, or nine of the biomarkers is altered relative to a control level of the respective biomarker. In some embodiments, the subject is identified as having a latent TB infection that is likely to transition into active TB disease if the level of one or more of MMP-1, C9, D-dimer, IP-10, IGFBP-2, MIG, and/or NPS is higher than a control level of the respective biomarker; and/or the levels of one or more of CA2D3, MMP-2, CD79A, PKB a/b/g, PGCB, PABP3, MXRA7, CNTFR alpha, Nr-CAM, Ephrin-A3, CD36 Ag, NDUB4, PCI, BOC, PKB beta, and/or JKIP3 is lower than a control level of the respective biomarker.

In some embodiments, a method comprises detecting the level of C9 and optionally one or more of IGFBP-2, CD79A, MXRA7, and NR-CAM in a sample from the subject. In some embodiments, a method comprises detecting the level of IGFBP-2 and optionally one or more of C9, CD79A, MXRA7, and NR-CAM in a sample from the subject. In some embodiments, a method comprises detecting the level of CD79A and optionally one or more of C9, IGFBP-2, MXRA7, and NR-CAM in a sample from the subject. In some embodiments, a method comprises detecting the level of MXRA7 and optionally one or more of C9, IGFBP-2, CD79A, and NR-CAM in a sample from the subject. In some embodiments, a method comprises detecting the level of NR-CAM and optionally one or more of C9, IGFBP-2, CD79A, and MXRA7 in a sample from the subject.

In some embodiments, detection of a particular level of a biomarker from Table 11 in a sample (e.g., plasma, serum, urine, saliva, etc.) from the subject that is altered relative to a control level of the respective biomarker is indicative of and/or diagnostic for a latent TB infection that is likely to develop into active TB disease within 30 days, 45 days, 60 days, 90 days, 120 days, 180 days, 270 days, 360 days, 450 days, or 540 days transitioning to active TB infection. In some embodiments, a level of at least one biomarker from Table 11 that is altered relative to the level of the respective biomarker in a control sample indicates that a subject with latent TB infection is likely to develop active TB disease. In some embodiments, the subject is identified as having a latent TB infection that is likely to transition into active TB disease if the level of one or more of MMP-1, C9, D-dimer, IP-10, IGFBP-2, MIG, and/or NPS is higher than a control level of the respective biomarker; and/or the levels of one or more of CA2D3, MMP-2, CD79A, PKB a/b/g, PGCB, PABP3, MXRA7, CNTFR alpha, Nr-CAM, Ephrin-A3, CD36 Ag, NDUB4, PCI, BOC, PKB beta, and/or JKIP3 is lower than a control level of the respective biomarker.

In some embodiments, provided herein are methods of determining a likelihood of a latent tuberculosis (TB) infection in a subject transitioning to active TB disease, comprising detecting the level of at least one, at least two, at least three, at least four, at least five, at least six, at least seven, or at least eight, biomarkers selected from the biomarkers in Table 11 in a sample from the subject, wherein the subject is identified as having a latent TB infection that is likely to develop into active TB disease within 30 days, 45 days, 60 days, 90 days, 120 days, 180 days, 270 days, 360 days, 450 days, or 540 days if the level of the respective biomarker is altered relative to a control level of the respective biomarker. In some embodiments, methods further comprise detecting the level of one or more biomarkers that are indicative of one or more of: the presence of latent TB infection, the presence of active TB disease, the strain of TB, the antibiotic resistance/sensitivity of TB, and/or the presence of other diseases. In some embodiments, methods comprise detecting the levels of 2 to 20 biomarkers, or 2 to 10 biomarkers, or 2 to 9 biomarkers, or 3 to 20 biomarkers, or 3 to 10 biomarkers, or 3 to 9 biomarkers, or 4 to 20 biomarkers, or 4 to 10 biomarkers, or 4 to 9 biomarkers, or 5 to 20 biomarkers, or 5 to 10 biomarkers, or 5 to 9 biomarkers.

In some embodiments, provided herein are methods of determining a likelihood of a latent TB infection in a subject transitioning to active TB disease, comprising detecting the levels of a set of biomarkers from Table A, Table B, or Table C in a sample from the subject, wherein the subject is identified as having a latent TB infection that is likely to develop into active TB disease within 30 days, 45 days, 60 days, 90 days, 120 days, 180 days, 270 days, 360 days, 450 days, or 540 days if the level of the respective biomarker is altered relative to a control level of the respective biomarker. In some embodiments, methods further comprise detecting the level of one or more biomarkers that are indicative of one or more of: the presence of latent TB infection, the presence of active TB disease, the strain of TB, the antibiotic resistance/sensitivity of TB, and/or the presence of other diseases. In some embodiments, methods comprise detecting the levels of 2 to 20 biomarkers, or 2 to 10 biomarkers, or 2 to 9 biomarkers, or 3 to 20 biomarkers, or 3 to 10 biomarkers, or 3 to 9 biomarkers, or 4 to 20 biomarkers, or 4 to 10 biomarkers, or 4 to 9 biomarkers, or 5 to 20 biomarkers, or 5 to 10 biomarkers, or 5 to 9 biomarkers.

In some embodiments, provided herein are methods of determining a likelihood of a latent tuberculosis (TB) infection in a subject transitioning to active TB disease, comprising detecting the level of at least one, at least two, at least three, at least four, or five biomarkers selected from C9, IGFBP-2, CD79A, MXRA7, and NR-CAM in a sample from the subject, wherein the subject is identified as having a latent TB infection that is likely to develop into active TB disease within 30 days, 45 days, 60 days, 90 days, 120 days, 180 days, 270 days, 360 days, 450 days, or 540 days if the level of the respective biomarker is altered relative to a control level of the respective biomarker. In some embodiments, methods further comprise detecting the level of one or more biomarkers that are indicative of one or more of: the presence of latent TB infection, the presence of active TB disease, the strain of TB, the antibiotic resistance/sensitivity of TB, and/or the presence of other diseases. In some embodiments, methods comprise detecting the levels of 2 to 20 biomarkers, or 2 to 10 biomarkers, or 2 to 9 biomarkers, or 3 to 20 biomarkers, or 3 to 10 biomarkers, or 3 to 9 biomarkers, or 4 to 20 biomarkers, or 4 to 10 biomarkers, or 4 to 9 biomarkers, or 5 to 20 biomarkers, or 5 to 10 biomarkers, or 5 to 9 biomarkers.

In various embodiments, the subject is identified as having a latent TB infection that is likely to transition into active TB disease within 30 days, 45 days, 60 days, 90 days, 120 days, 180 days, 270 days, 360 days, 450 days, or 540 days if the level of one or more of MMP-1, C9, D-dimer, IP-10, IGFBP-2, MIG, and/or NPS is higher than a control level of the respective biomarker; and/or the levels of one or more of CA2D3, MMP-2, CD79A, PKB a/b/g, PGCB, PABP3, MXRA7, CNTFR alpha, Nr-CAM, Ephrin-A3, CD36 Ag, NDUB4, PCI, BOC, PKB beta, and/or JKIP3 is lower than a control level of the respective biomarker.

In some embodiments, provided herein are methods of monitoring a latent TB infection in a subject for the likelihood of the latent TB infection transitioning to active TB disease, comprising detecting the level of at least one, at least two, at least three, at least four, at least five, at least six, at least seven, or at least eight, biomarkers selected from the biomarkers in Table 11 in a sample from the patient at a first time point, and measuring the level of the at least one, at least two, at least three, at least four, at least five, at least six, at least seven, or eight biomarkers at a second time point. In some embodiments, if the level of the biomarkers is further from a control level at the second time point than the first time point, the likelihood of the latent TB infection transitioning to active TB disease has increased. In some embodiments, if the level of the biomarkers is further from a control level at the second time point than at the first time point, the likelihood of the latent TB infection transitioning to active TB disease has increased. In some embodiments, if the level of the biomarkers is closer to a control level at the second time point than at the first time point, the likelihood of the latent TB infection transitioning to active TB disease has decreased.

In some embodiments, provided herein are methods of monitoring a latent TB infection in a subject for the likelihood of the latent TB infection transitioning to active TB disease, comprising detecting the levels of a set of biomarkers from Table A, Table B, or Table C in a sample from the patient at a first time point, and measuring the level of the at least one, at least two, at least three, at least four, at least five, at least six, at least seven, at least eight, or nine biomarkers at a second time point. In some embodiments, if the level of the biomarkers is further from a control level at the second time point than the first time point, the likelihood of the latent TB infection transitioning to active TB disease within 30 days, 45 days, 60 days, 90 days, 120 days, 180 days, 270 days, 360 days, 450 days, or 540 days has increased.

In various embodiments, if the level of one or more of MMP-1, C9, D-dimer, IP-10, IGFBP-2, MIG, and/or NPS is higher at the second time point than at the first time point; and/or the level of one or more of CA2D3, MMP-2, CD79A, PKB a/b/g, PGCB, PABP3, MXRA7, CNTFR alpha, Nr-CAM, Ephrin-A3, CD36 Ag, NDUB4, PCI, BOC, PKB beta, and/or JKIP3 is lower at the second time point than at the first time point, the likelihood of the latent TB infection transitioning to active TB disease has increased. In some embodiments, if the level of one or more of MMP-1, C9, D-dimer, IP-10, IGFBP-2, MIG, and/or NPS is lower at the second time point than at the first time point; and/or the level of one or more of CA2D3, MMP-2, CD79A, PKB a/b/g, PGCB, PABP3, MXRA7, CNTFR alpha, Nr-CAM, Ephrin-A3, CD36 Ag, NDUB4, PCI, BOC, PKB beta, and/or JKIP3 is higher at the second time point than at the first time point, the likelihood of the latent TB infection transitioning to active TB disease has decreased.

In some embodiments, provided herein are methods of monitoring a latent TB infection in a subject for the likelihood of the latent TB infection transitioning to active TB disease, comprising detecting the level of at least one, at least two, at least three, at least four, or five biomarkers selected from C9, IGFBP-2, CD79A, MXRA7, and NR-CAM in a sample from the patient at a first time point, and measuring the level of the at least one, at least two, at least three, at least four, at least five, at least six, at least seven, at least eight, or nine biomarkers at a second time point. In some embodiments, if the level of the biomarkers is further from a control level at the second time point than the first time point, the likelihood of the latent TB infection transitioning to active TB disease within 30 days, 45 days, 60 days, 90 days, 120 days, 180 days, 270 days, 360 days, 450 days, or 540 days has increased. In some embodiments, if the level of C9 and/or IGFBP-2 is higher at the second time point than at the first time point, and/or the level of CD79A, MXRA7, and/or NR-CAM is lower at the second time point than at the first time point, the likelihood of the latent TB infection transitioning to active TB disease has increased. In some embodiments, if the level of the biomarkers is nearer to a control level at the second time point than the first time point, the likelihood of the latent TB infection transitioning to active TB disease has decreased. In some embodiments, if the level of C9 and/or IGFBP-2 is lower at the second time point than at the first time point, and/or the level of CD79A, MXRA7, and/or NR-CAM is higher at the second time point than at the first time point, the likelihood of the latent TB infection transitioning to active TB disease has decreased.

In some embodiments, provided herein are methods of monitoring treatment of a latent TB infection, comprising detecting the level of at least one, at least two, at least three, at least four, at least five, at least six, at least seven, or at least eight, biomarkers selected from the biomarkers in Table 11 in a sample from the patient at a first time point, administering at least one treatment for TB infection to the patient, and detecting the levels of the respective biomarkers in a sample from the patient at a second time point, wherein the treatment is effective at reducing the likelihood of the latent TB infection transitioning to active TB disease if the level of the biomarkers is nearer to a control level at the second time point compared to the first time point. In some embodiments, provided herein are methods of monitoring treatment of a latent TB infection, comprising detecting the levels of a set of biomarkers from Table A, Table B, or Table C in a sample from the patient at a first time point, administering at least one treatment for TB infection to the patient, and detecting the levels of the respective biomarkers in a sample from the patient at a second time point. In some embodiments, the treatment is effective at reducing the likelihood of the latent TB infection transitioning to active TB disease if the level of the biomarkers is nearer to a control level, or is not further from a control level than, at the second time point compared to the first time point. In some embodiments, provided herein are methods of monitoring treatment of a latent TB infection, comprising detecting the level of at least one, at least two, at least three, at least four, or five biomarkers selected from C9, IGFBP-2, CD79A, MXRA7, and NR-CAM in a sample from the patient at a first time point, administering at least one treatment for TB infection to the patient, and detecting the levels of the respective biomarkers in a sample from the patient at a second time point. In some embodiments, the treatment is effective at reducing the likelihood of the latent TB infection transitioning to active TB disease if the level of the biomarkers is nearer to a control level, or is not further from a control level than, at the second time point compared to the first time point.

In some embodiments, the treatment is effective at reducing the likelihood of the latent TB infection transitioning to active TB disease if the level of one or more of MMP-1, C9, D-dimer, IP-10, IGFBP-2, MIG, and/or NPS is lower at the second time point than at the first time point; and/or the level of one or more of CA2D3, MMP-2, CD79A, PKB a/b/g, PGCB, PABP3, MXRA7, CNTFR alpha, Nr-CAM, Ephrin-A3, CD36 Ag, NDUB4, PCI, BOC, PKB beta, and/or JKIP3 is higher at the second time point than at the first time point. In some embodiments, the at least one treatment for TB infection is selected from the group consisting of isoniazid (INH), rifampin (RIF), rifapentine (RPT), ethambutol (EMB), pyrazinamide (PZA), and/or another approved TB therapeutic to the subject.

In some embodiments, a control level is the level of the respective biomarker in a subject or population of subjects with latent TB infection who are known not to have developed active TB within a particular time period. In some embodiments, a control level is the level of the respective biomarker in a subject or population of subjects with latent TB infection who are known not to have developed active TB within 540 days of sample collection. In some embodiments, a control level is the level of the respective biomarker in a subject or population of subjects with latent TB infection who are known not to have developed active TB within 2 years of sample collection.

In some embodiments, methods further comprise performing one or more additional tests for TB infection. In some embodiments, additional tests for TB infection comprise chest x-ray.

In some methods described herein, each biomarker is a protein biomarker. In some embodiments, methods comprise contacting biomarkers of the sample from the subject or patient with a set of biomarker capture reagents, wherein each biomarker capture reagent of the set of biomarker capture reagents specifically binds to a different biomarker being detected. In some embodiments, each biomarker capture reagent is an antibody or an aptamer. In some embodiments, at least one aptamer is a slow off-rate aptamer. In some embodiments, at least one slow off-rate aptamer comprises at least one, at least two, at least three, at least four, at least five, at least six, at least seven, at least eight, at least nine, or at least 10 nucleotides with modifications. In some embodiments, each slow off-rate aptamer binds to its target protein with an off rate (t½) of ≥30 minutes, ≥60 minutes, ≥90 minutes, ≥120 minutes, ≥150 minutes, ≥180 minutes, ≥210 minutes, or ≥240 minutes.

In some embodiments, the sample is a blood sample. In some embodiments, the sample is a serum sample.

In some embodiments, a method for determining whether a latent TB infection is likely to advance into active TB disease in a subject within 30 days, 45 days, 60 days, 90 days, 120 days, 180 days, 270 days, 360 days, 450 days, or 540 days comprises (a) forming a biomarker panel having N biomarker proteins selected from the biomarkers in Table 11; and (b) detecting the level of each of the N biomarker proteins of the panel in a sample from the subject. In some embodiments, N is 1 to 9. In some embodiments, N is 2 to 9. In some embodiments, N is 3 to 9. In some embodiments, N is 4 to 9. In some embodiments, N is 5 to 9. In some embodiments, N is 6 to 9. In some embodiments, N is 7 to 9. In some embodiments, N is 8 to 9. In some embodiments, N is 9. In some embodiments, N is 2 to 8. In some embodiments, N is 3 to 7. In some embodiments, N is 4 to 6. In some embodiments, N is 1 to 8. In some embodiments, N is 2 to 8. In some embodiments, N is 3 to 8. In some embodiments, N is 4 to 8. In some embodiments, N is 4 to 6. In some embodiments, N is 4 to 5.

In some embodiments, a set of biomarker proteins with an AUC_0-180 value of 0.94 or greater, or 0.95 or greater, or 0.96 or greater, or 0.97 or greater, or 0.98 or greater is selected from Table A. In some embodiments, a set of biomarker proteins with an AUC_180-360 value of 0.76 or greater, or 0.77 or greater, or 0.78 or greater, or 0.79 or greater, or 0.80 or greater is selected from Table A. In some embodiments, a set of biomarker proteins with an AUC_0-180 value of 0.93 or greater, or 0.94 or greater, or 0.95 or greater, or 0.96 or greater, or 0.97 or greater, or 0.98 or greater is selected from Table B. In some embodiments, a set of biomarker proteins with an AUC180-360 value of 0.76 or greater, or 0.77 or greater, or 0.78 or greater, or 0.79 or greater, or 0.80 or greater, or 0.81 or greater, 0.82 or greater is selected from Table B. In some embodiments, a set of biomarker proteins with an AUC0-180 value of 0.93 or greater, or 0.94 or greater, or 0.95 or greater, or 0.96 or greater, or 0.97 or greater is selected from Table C. In some embodiments, a set of biomarker proteins with an AUC180-360 value of 0.80 or greater, or 0.81 or greater, 0.82 or greater is selected from Table C.

In any of the embodiments described herein, the method may comprise detecting the levels of C9 and at least one, at least two, or three biomarkers selected from PABP3, IGFBP-2, D-dimer, MXRA7, IP-10, CD79A, MMP-2, CA2D3, NDUB4, PKB beta, PKB a/b/g, CNTFR alphA, JKIP3 and Nr-CAM. In any of the embodiments described herein, the method may comprise detecting the levels of IP-10 and at least one, at least two, or three biomarkers selected from CA2D3, PKB a/b/g, CD79A, PABP3, MXRA7 and NDUB4. In any of the embodiments described herein, the method may comprise detecting the levels of D-dimer and at least one, at least two, or three biomarkers selected from CD79A, IP-10, IGFBP-2, CA2D3, MMP-2, MXRA7, PABP3, PKB a/b/g, NDUB4, PCI, JKIP3 and CD36 antigen.

In any of the embodiments described herein, the method may comprise detecting a set of biomarkers selected from D-dimer, CD79A, MXRA7 and NDUB4; D-dimer, IP-10, CA2D3 and MXRA7; C9, PABP3, MXRA7 and NDUB4; C9, IGFBP-2, CA2D3 and MXRA7; C9, D-dimer, IP-10 and PABP3; C9, MXRA7, NDUB4 and JKIP3; C9, D-dimer, PABP3 and MXRA7; D-dimer, CD79A, PABP3 and PCI; C9, D-dimer, MXRA7 and NDUB4; D-dimer, IP-10, IGFBP-2 and MXRA7; D-dimer, IP-10, IGFBP-2 and PABP3; C9, D-dimer, IP-10 and MXRA7; D-dimer, IP-10, MXRA7 and JKIP3; D-dimer, IGFBP-2, CA2D3 and MXRA7; C9, IP-10, MXRA7 and NDUB4; C9, D-dimer, MXRA7 and Nr-CAM; C9, IP-10, MXRA7 and JKIP3; and IP-10, CA2D3, MXRA7 and NDUB4.

In any of the embodiments described herein, the method may comprise detecting the levels of C9 and at least one, at least two, at least three, or four biomarkers selected from D-dimer, IP-10, MMP-2, PABP3, IGFBP-2, CA2D3, PKB a/b/g, NDUB4, CD79A, NPS, CD36 ANTIGEN, MXRA7, CNTFR alpha, JKIP3, Nr-CAM, PCI and BOC.

In any of the embodiments described herein, the method may comprise detecting the levels of IP-10 and at least one, at least two, at least three, or four biomarkers selected from CA2D3, IGFBP-2, PKB a/b/g, CD79A, MMP-2, NPS, PABP3, MXRA7, CNTFR alpha, NDUB4, Nr-CAM, CD36 ANTIGEN, PCI, PKB beta, Ephrin-A3 and JKIP3.

In any of the embodiments described herein, the method may comprise detecting the levels of D-dimer and at least one, at least two, at least three, or four biomarkers selected from IGFBP-2, IP-10, CD79A, CA2D3, MXRA7, PABP3, CD36 ANTIGEN, PKB a/b/g, MMP-2, NPS, CNTFR alpha, Nr-CAM, NDUB4, PGCB, JKIP3, PCI and BOC.

In any of the embodiments described herein, the method may comprise detecting the levels of MMP-1 and at least one, at least two, at least three, or four biomarkers selected from IP-10, C9, PKB a/b/g, IGFBP-2, PABP3, CA2D3, PGCB, NDUB4, CD79A, MXRA7, CD36 ANTIGEN, CNTFR alpha, BOC, PKB beta, JKIP3 and PCI.

In any of the embodiments described herein, the method may comprise detecting a set of biomarkers selected from C9, D-dimer, IGFBP-2, MMP-2 and MXRA7; C9, D-dimer, PABP3, MXRA7 and NDUB4; C9, D-dimer, IGFBP-2, CA2D3 and MXRA7; MMP-1, IP-10, IGFBP-2, PABP3 and MXRA7; C9, D-dimer, MMP-2, MXRA7 and NDUB4; C9, D-dimer, MMP-2, PABP3 and MXRA7; D-dimer, IGFBP-2, CA2D3, PABP3 and MXRA7; MMP-1, IP-10, IGFBP-2, MXRA7 and NDUB4; C9, D-dimer, MMP-2, MXRA7 and JKIP3; MMP-1, C9, IP-10, MXRA7 and BOC; C9, D-dimer, MXRA7, NDUB4 and JKIP3; MMP-1, C9, IP-10, MXRA7 and CNTFR alpha; D-dimer, IP-10, IGFBP-2, CA2D3 and PABP3; C9, IGFBP-2, PABP3, MXRA7 and Nr-CAM; C9, IP-10, MXRA7, NDUB4 and JKIP3; C9, D-dimer, MMP-2, PABP3 and NDUB4; MMP-1, IP-10, PABP3, MXRA7 and NDUB4; C9, D-dimer, PABP3, MXRA7 and Nr-CAM; MMP-1, IP-10, IGFBP-2, CA2D3 and MXRA7; C9, MMP-2, PABP3, MXRA7 and NDUB4; C9, D-dimer, IP-10, CA2D3 and MXRA7; MMP-1, IP-10, PKB a/b/g, PABP3 and MXRA7; MMP-1, IP-10, CA2D3, PABP3 and MXRA7; D-dimer, IGFBP-2, CA2D3, CD79A and MXRA7; MMP-1, IP-10, IGFBP-2, PABP3 and CD36 ANTIGEN; D-dimer, IP-10, IGFBP-2, PABP3 and CNTFR alpha; MMP-1, IP-10, IGFBP-2, MXRA7 and CNTFR alpha; C9, D-dimer, IP-10, PABP3 and Nr-CAM; C9, IP-10, CA2D3, PKB a/b/g and MXRA7; D-dimer, IP-10, IGFBP-2, PABP3 and CD36 ANTIGEN; C9, IP-10, PKB a/b/g, MXRA7 and NDUB4; D-dimer, IP-10, IGFBP-2, PABP3 and JKIP3; C9, D-dimer, IP-10, PKB a/b/g and MXRA7; D-dimer, IP-10, IGFBP-2, CA2D3 and JKIP3; MMP-1, IP-10, IGFBP-2, CD36 ANTIGEN and PKB beta; MMP-1, IP-10, IGFBP-2, PGCB and MXRA7; D-dimer, IP-10, IGFBP-2, PABP3 and NDUB4; C9, PABP3, NDUB4, PKB beta and JKIP3; C9, IGFBP-2, CD79A, PABP3 and MXRA7; C9, D-dimer, NPS, MMP-2 and MXRA7; C9, D-dimer, IGFBP-2, MXRA7 and Nr-CAM; C9, D-dimer, IP-10, CA2D3 and PKB a/b/g; C9, CA2D3, CD36 ANTIGEN, PKB beta and JKIP3; C9, D-dimer, CA2D3, PABP3 and JKIP3; C9, D-dimer, IP-10, PABP3 and JKIP3; C9, D-dimer, MMP-2, PKB a/b/g and MXRA7; MMP-1, IP-10, PABP3, MXRA7 and PCI; C9, D-dimer, IGFBP-2, PABP3 and MXRA7; MMP-1, IP-10, PABP3, MXRA7 and CNTFR alpha; C9, D-dimer, CA2D3, MXRA7 and JKIP3; C9, IP-10, PABP3, MXRA7 and NDUB4; MMP-1, IP-10, IGFBP-2, PKB a/b/g and MXRA7; MMP-1, IP-10, IGFBP-2, PABP3 and PCI; C9, CA2D3, MXRA7, NDUB4 and JKIP3; IP-10, CA2D3, PABP3, CNTFR alpha and NDUB4; C9, D-dimer, PABP3, MXRA7 and JKIP3; C9, D-dimer, IP-10, MXRA7 and JKIP3; C9, D-dimer, CA2D3, PKB a/b/g and MXRA7; MMP-1, IP-10, IGFBP-2, MXRA7 and PKB beta; MMP-1, IP-10, IGFBP-2, MXRA7 and PCI; MMP-1, IP-10, CA2D3, MXRA7 and NDUB4; C9, D-dimer, IGFBP-2, CNTFR alpha and BOC; D-dimer, IP-10, MXRA7, CD36 ANTIGEN and JKIP3; C9, D-dimer, IP-10, MMP-2 and NDUB4; C9, PABP3, MXRA7, NDUB4 and JKIP3; MMP-1, IP-10, IGFBP-2, PABP3 and NDUB4; C9, IGFBP-2, CA2D3, PABP3 and MXRA7; D-dimer, IP-10, CA2D3, MXRA7 and CD36 ANTIGEN; D-dimer, IP-10, IGFBP-2, CD79A and NDUB4; C9, D-dimer, PKB a/b/g, MXRA7 and NDUB4; C9, D-dimer, MXRA7, CNTFR alpha and CD36 ANTIGEN; MMP-1, IP-10, PGCB, MXRA7 and CNTFR alpha; IP-10, IGFBP-2, CD79A, PABP3 and CNTFR alpha; MMP-1, PKB a/b/g, NDUB4, BOC and PKB beta; C9, PABP3, MXRA7, CNTFR alpha and CD36 ANTIGEN; C9, D-dimer, IGFBP-2, MXRA7 and NDUB4; D-dimer, IP-10, IGFBP-2, MXRA7 and NDUB4; C9, D-dimer, IP-10, MXRA7 and NDUB4; C9, D-dimer, IGFBP-2, CD79A and CNTFR alpha; D-dimer, IP-10, CA2D3, PABP3 and CD36 ANTIGEN; C9, D-dimer, PABP3, NDUB4 and BOC; C9, D-dimer, CA2D3, MXRA7 and PKB beta; C9, IGFBP-2, CA2D3, CD79A and MXRA7; C9, D-dimer, CA2D3, MXRA7 and Nr-CAM; C9, D-dimer, IP-10, PABP3 and NDUB4; MMP-1, IP-10, PKB a/b/g, MXRA7 and PKB beta; IP-10, IGFBP-2, PABP3, CNTFR alpha and PCI; C9, IP-10, PABP3, MXRA7 and CNTFR alpha; C9, D-dimer, IGFBP-2, MXRA7 and BOC; C9, IP-10, CA2D3, MXRA7 and NDUB4; C9, D-dimer, PKB a/b/g, PABP3 and MXRA7; C9, IP-10, MMP-2, PABP3 and NDUB4; D-dimer, IP-10, PABP3, CNTFR alpha and CD36 ANTIGEN; IP-10, IGFBP-2, PABP3, NDUB4 and PCI; C9, IP-10, IGFBP-2, PABP3 and MXRA7; C9, D-dimer, IP-10, CD36 ANTIGEN and BOC; MMP-1, IP-10, IGFBP-2, PABP3 and CNTFR alpha; IP-10, IGFBP-2, CA2D3, PABP3 and MXRA7; C9, D-dimer, IP-10, CA2D3 and NDUB4; IP-10, PKB a/b/g, PABP3, Nr-CAM and PKB beta; IP-10, CD79A, PKB a/b/g, PABP3 and PKB beta; MMP-1, IP-10, PABP3, MXRA7 and JKIP3; MMP-1, IP-10, CD79A, PABP3 and MXRA7; MMP-1, C9, IP-10, PKB a/b/g and MXRA7; C9, D-dimer, IP-10, MXRA7 and Nr-CAM; C9, D-dimer, IP-10, IGFBP-2 and MXRA7; D-dimer, IP-10, CA2D3, MXRA7 and JKIP3; D-dimer, IGFBP-2, CA2D3, PABP3 and PCI; and C9, IP-10, IGFBP-2, PABP3 and Nr-CAM.

In any of the embodiments described herein, the method may comprise detecting a set of biomarkers selected from MMP-1, IP-10, IGFBP-2, PABP3 and MXRA7; C9, D-dimer, MMP-2, PABP3 and MXRA7; MMP-1, IP-10, IGFBP-2, MXRA7 and NDUB4; MMP-1, IP-10, PABP3, MXRA7 and NDUB4; MMP-1, IP-10, IGFBP-2, CA2D3 and MXRA7; MMP-1, IP-10, PKB a/b/g, PABP3 and MXRA7; MMP-1, IP-10, CA2D3, PABP3 and MXRA7; MMP-1, IP-10, IGFBP-2, PABP3 and CD36 ANTIGEN; MMP-1, IP-10, IGFBP-2, MXRA7 and CNTFR alpha; C9, IP-10, CA2D3, PKB a/b/g and MXRA7; MMP-1, IP-10, PABP3, MXRA7 and PCI; MMP-1, IP-10, PABP3, MXRA7 and CNTFR alpha; MMP-1, IP-10, IGFBP-2, PKB a/b/g and MXRA7; MMP-1, IP-10, IGFBP-2, PABP3 and PCI; MMP-1, IP-10, IGFBP-2, MXRA7 and PCI; MMP-1, IP-10, CA2D3, MXRA7 and NDUB4; MMP-1, IP-10, IGFBP-2, PABP3 and NDUB4; MMP-1, IP-10, PGCB, MXRA7, CNTFR alpha; IP-10, IGFBP-2, PABP3, NDUB4 and PCI; MMP-1, IP-10, PABP3, MXRA7 and JKIP3; MMP-1, IP-10, CD79A, PABP3 and MXRA7; and C9, D-dimer, IP-10, MXRA7 and Nr-CAM.

In some embodiments, one or more additional steps are taken upon identifying a subject as having a latent TB infection that is likely to transition into active TB disease within 30 days, 45 days, 60 days, 90 days, 120 days, 180 days, 270 days, 360 days, 450 days, or 540 days. In some embodiments, methods further comprise a subsequent step of treating said subject or patient for latent TB. In some embodiments, methods further comprise a subsequent step of treating said subject or patient for active TB disease. In some embodiments, methods further comprise a subsequent step of additional TB-diagnostic steps. In some embodiments, said additional TB-diagnostic steps comprise a chest x-ray. In some embodiments, methods further comprise generating a report indicating that said subject is likely to develop active TB disease within 30 days, 45 days, 60 days, 90 days, 120 days, 180 days, 270 days, 360 days, 450 days, or 540 days. In some embodiments, a subject with such a risk is treated for active TB disease before developing symptoms of active TB disease.

In any of the embodiments described herein, the each biomarker may be a protein biomarker. In any of the embodiments described herein, the method may comprise contacting biomarkers of the sample from the subject or patient with a set of biomarker detection reagents. In any of the embodiments described herein, the method may comprise contacting biomarkers of the sample from the subject or patient with a set of biomarker capture reagents, wherein each biomarker capture reagent of the set of biomarker capture reagents specifically binds to a biomarker being detected. In some embodiments, each biomarker capture reagent of the set of biomarker capture reagents specifically binds to a different biomarker being detected. In any of the embodiments described herein, each biomarker capture reagent may be an antibody or an aptamer. In any of the embodiments described herein, each biomarker capture reagent may be an aptamer. In any of the embodiments described herein, at least one aptamer may be a slow off-rate aptamer. In any of the embodiments described herein, at least one slow off-rate aptamer may comprise at least one, at least two, at least three, at least four, at least five, at least six, at least seven, at least eight, at least nine, or at least 10 nucleotides with modifications. In some embodiments, the modifications are hydrophobic modifications. In some embodiments, the modifications are hydrophobic base modifications. In some embodiments, one or more of the modifications may be selected from the modifications shown in FIG. 50. In some embodiments, each slow off-rate aptamer binds to its target protein with an off rate (t½) of ≥30 minutes, ≥60 minutes, ≥90 minutes, ≥120 minutes, ≥150 minutes, ≥180 minutes, ≥210 minutes, or ≥240 minutes.

In any of the embodiments described herein, the sample may be a blood sample. In some embodiments, the blood sample is selected from a serum sample and a plasma sample. In some embodiments, the sample is a body fluid selected from tracheal aspirate fluid, bronchoalveolar fluid, bronchoalveolar lavage sample, blood or portion thereof, serum, plasma, urine, semen, saliva, tears, etc.

In any of the embodiments described herein, a method may further comprise treating the subject or patient for TB infection or TB disease. In some embodiments, treating the subject or patient for TB infection or TB disease comprises a treatment regimen of administering one or more of: isoniazid (INH), rifampin (RIF), rifapentine (RPT), ethambutol (EMB), pyrazinamide (PZA), and/or another approved TB therapeutic to the subject or patient.

In some embodiments, kits are provided. In some embodiments, a kit comprises at least one, at least two, at least three, at least four, at least five, at least six, at least seven, at least eight, or nine aptamers, wherein each aptamer specifically binds to a target protein selected from the target proteins in Table 11. In some embodiments, the kit comprises a total of 2 to 9 aptamers, or 3 to 9 aptamers, or 4 to 9 aptamers, or 4 to 8 aptamers, or 4 to 7 aptamers, or 4 to 6 aptamers, or 4 to 5 aptamers, or 4 aptamers, or 5 aptamers, or 6 aptamers, or 7 aptamers, or 8 aptamers, or 9 aptamers. In some embodiments, aptamer specifically binds to a target protein of a set of target proteins from Table A, Table B, or Table C. In some embodiments, each aptamer specifically binds to a target protein of a set of target proteins from Table C. In some embodiments, each aptamer specifically binds to a target protein selected from C9, IGFBP-2, CD79A, MXRA7, and NR-CAM. In some embodiments, the kit comprises 5 aptamers, wherein each aptamer selectively binds a target protein selected from C9, IGFBP-2, CD79A, MXRA7, and NR-CAM.

In some embodiments, the kit comprises a total of 2 to 20 aptamers, or 2 to 10 aptamers, or 2 to 9 aptamers, or 3 to 20 aptamers, or 3 to 10 aptamers, or 3 to 9 aptamers, or 4 to 20 aptamers, or 4 to 10 aptamers, or 4 to 9 aptamers, or 5 to 20 aptamers, or 5 to 10 aptamers, or 5 to 9 aptamers. In some embodiments, a kit comprises X aptamers, wherein N aptamers specifically bind to a biomarker protein selected from Table 11. In some embodiments, X is less than 100 (e.g., <90, <80, <70, <60, <50, <40, <30, <20, <15). In some embodiments, X is 10 or more (e.g., >10, >11, >12, >13, >14, >15, >20, >30, >40, >50). In some embodiments, X is between 10 and 100, between 10 and 90, between 10 and 80, between 10 and 70, between 10 and 60, between 10 and 50, between 10 and 40, between 10 and 30, between 10 and 20, or between 10 and 15. In some embodiments, N is 1 to 9 (1, 2, 3, 4, 5, 6, 7, 8, 9). In some embodiments, N is 1 to 8 (1, 2, 3, 4, 5, 6, 7, 8).

In some embodiments, compositions are provided comprising proteins of a sample from a subject or patient and at least one, at least two, at least three, at least four, at least five, at least six, at least seven, at least eight, or nine aptamers, wherein each aptamer specifically binds to a target protein selected from the target proteins in Table 11. In some embodiments, the composition comprises a total of 2 to 9 aptamers, or 3 to 9 aptamers, or 4 to 9 aptamers, or 4 to 8 aptamers, or 4 to 7 aptamers, or 4 to 6 aptamers, or 4 to 5 aptamers, or 4 aptamers, or 5 aptamers, or 6 aptamers, or 7 aptamers, or 8 aptamers, or 9 aptamers. In some embodiments, each aptamer specifically binds to a target protein of a set of target proteins from Table A, Table B, or Table C. In some embodiments, each aptamer specifically binds to a target protein of a set of target proteins from Table C. In some embodiments, each aptamer specifically binds to a target protein selected from C9, IGFBP-2, CD79A, MXRA7, and NR-CAM. In some embodiments, the composition comprises 5 aptamers, wherein each aptamer selectively binds a target protein selected from C9, IGFBP-2, CD79A, MXRA7, and NR-CAM.

In any of the embodiments described herein, a kit or composition may comprise at least one aptamer that is a slow off-rate aptamer. In any of the embodiments described herein, each aptamer of a kit or composition may be a slow off-rate aptamer. In some embodiments, at least one slow off-rate aptamer comprises at least one, at least two, at least three, at least four, at least five, at least six, at least seven, at least eight, at least nine, or at least 10 nucleotides with modifications. In some embodiments, at least one nucleotide with a modification is a nucleotide with a hydrophobic base modification. In some embodiments, each nucleotide with a modification is a nucleotide with a hydrophobic base modification. In some embodiments, each hydrophobic base modification is independently selected from the modification in FIG. 50. In some embodiments, each slow off-rate aptamer in a kit binds to its target protein with an off rate (t½) of ≥30 minutes, ≥60 minutes, ≥90 minutes, ≥120 minutes, ≥150 minutes, ≥180 minutes, ≥210 minutes, or ≥240 minutes.

### BRIEF DESCRIPTION OF THE DRAWINGS

FIG. 1 shows (left) beeswarm plots of sampling time for all cases in the discovery and verification sets for all time points, and (right) empirical cumulative distribution functions of time to diagnosis in discovery and verification.

FIG. 2 shows boxplots of log2 transformed hybridization normalization scale factors for each plate (left), and cumulative distribution functions of raw normalization scale factors for each plate (right).

FIG. 3 shows boxplots of Log2 transformed median normalization scale factors.

FIG. 4 shows a subspace projection for each sample from a PCA performed using the top 50 ranked proteins which were observed to differentiate gender.

FIG. 5 shows a plot of the empirical CDF of age (top) and a demographic table (bottom) for all TB Cases and Controls, 0 to 950 to beginning of treatment.

FIG. 6 shows empirical CDFs for the top 9 ranked proteins comparing all TB case and all Control samples.

FIG. 7 shows a plot of KS distances with class randomization statistics for the top 100 features.

FIG. 8 shows a volcano plot of 3040 proteins from a univariate KS analysis comparing all TB Cases and all Controls.

FIG. 9 shows RFU trajectories of individual TB cases overlaid onto a ‘control band’ created by interpolating the median, IQR and range of the control data. The top axis corresponds to the controls and the bottom the TB cases. Time moves to the right.

FIG. 10 shows a heat map of t-statistics arranged by hierarchical clustering for the top 200 t-statistics ranked by the median across all bins.

FIG. 11 shows a heat map of t-statistics (left) and corresponding CDFs of cases and controls for subcluster A, which was selected based on inconsistencies in the 3TB/8Controls bin.

FIG. 12 shows a heat map of t-statistics (left) and corresponding CDFs of cases and controls for subcluster B, which was selected based on inconsistencies in the 4TB/6Controls bin.

FIG. 13 shows a heat map of t-statistics (left) and corresponding CDFs of cases and controls for subcluster C, which was selected because most proteins seemed to be homogenously higher in the TB cases.

FIG. 14 shows a heat map of t-statistics (left) and corresponding CDFs of cases and controls for subcluster D, which was selected based on most proteins being homogenously lower in the TB Cases.

FIG. 15 shows a heat map of t-statistics (left) and corresponding CDFs of cases and controls for subcluster E, which was selected based on inconsistencies in several bins.

FIG. 16 shows Linear fits for all TB cases are shown as a function of time to treatment. The dark band corresponds to the interquartile range (IQR), while the lighter shaded region corresponds to the whiskers, or the nearest data point that's within the upper/lower quartile+1.5*IQR. Data outside this range is considered an outlier.

FIG. 17 shows sample times for all TB subjects as a function of time to the beginning of treatment. Negative values are days on treatment.

FIG. 18 shows a plot of the empirical CDF of age (left) and a demographic table (right) for TB Cases 0-180 days to beginning of treatment, and matched Controls.

FIG. 19 shows empirical CDFs for the top 9 ranked proteins comparing non-TB vs. TB 0-180 days before treatment.

FIG. 20 shows a KS Plot of KS distances with class randomization statistics for the top 50 features comparing non-TB vs. TB 0-180 days before treatment (pvalue threshold=1.12e-3).

FIG. 21 shows a volcano plot of 3040 proteins from a univariate KS analysis comparing TB Cases 0-180 days pre-treatment to matched controls.

FIG. 22 shows RFU trajectories for the top markers found to distinguish non-TB vs. TB 0-180 days before treatment. Individual TB cases were overlaid onto a ‘control band’ created by interpolating the median, IQR and range of the control data. The top axis corresponds to the controls and the bottom to the TB cases. Time moves to the right.

FIG. 23 shows a plot of the empirical CDF of age (left) and a demographic table (right) for TB Cases 180-360 days to beginning of treatment and matched Controls

FIG. 24 shows empirical CDFs for the top 9 ranked proteins comparing non-TB vs. TB 180-360 days before treatment.

FIG. 25 shows a KS Plot of KS distances with class randomization statistics for the top 50 features comparing non-TB vs. TB 180-360 days before treatment (pvalue threshold=8.55e-3).

FIG. 26 shows a volcano plot of 3040 proteins from a univariate KS analysis comparing TB Cases 180-360 days pre-treatment to matched controls.

FIG. 27 shows RFU trajectories for the top markers found to distinguish non-TB vs. TB 180-360 days before treatment. Individual TB cases were overlaid onto a ‘control band’ created by interpolating the median, IQR and range of the control data. The top axis corresponds to the controls and the bottom the TB cases. Time moves to the right.

FIG. 28 shows a plot of the empirical CDF of age (left) and a demographic table (right) for TB Cases 360-540 days to beginning of treatment and matched Controls

FIG. 29 shows empirical CDFs for the top 9 ranked proteins comparing non-TB vs. TB 360-540 days before treatment.

FIG. 30 shows plot of KS distances with class randomization statistics for the top 50 features comparing non-TB vs. TB 360-540 days before treatment (pvalue threshold=3.84-3).

FIG. 31 shows a volcano plot of 3040 proteins from a univariate KS analysis comparing TB Cases 360-540 days pre-treatment to matched controls.

FIG. 32 shows RFU trajectories for the top markers found to distinguish non-TB vs. TB 360-540 days before treatment. Individual TB cases were overlaid onto a ‘control band’ created by interpolating the median, IQR and range of the control data. The top axis corresponds to the controls and the bottom the TB cases. Time moves to the right.

FIG. 33 shows a plot of the empirical CDF of age (left) and a demographic table (right) for TB Cases 540-700 days to beginning of treatment and matched Controls.

FIG. 34 shows empirical CDFs for the top 6 ranked proteins comparing non-TB vs. TB 540-700 days before treatment.

FIG. 35 shows a plot of KS distances with class randomization statistics for the top 50 features comparing non-TB vs. TB 540-700 days before treatment (pvalue threshold=2.5e-2).

FIG. 36 shows a volcano plot of 3040 proteins from a univariate KS analysis comparing TB Cases 540-700 days pre-treatment to matched controls.

FIG. 37 shows RFU trajectories for the top markers found to distinguish non-TB vs. TB 540-700 days before treatment. Individual TB cases were overlaid onto a ‘control band’ created by interpolating the median, IQR and range of the control data. The top axis corresponds to the controls and the bottom the TB cases. Time moves to the right.

FIG. 38 shows stability paths (top) and regularization paths (bottom) for non-TB Controls vs. TB Cases 0-180 days pre-treatment.

FIG. 39 shows empirical CDFs for the top 11 proteins whose maximum selection probability exceeded 50%.

FIG. 40 shows boxplots of log10 RFU versus binned time to treatment. The median and IQR of the controls are extended across the figure.

FIG. 41 shows performance 0-360 days before diagnosis versus binned time responsiveness.

FIG. 42 shows correlation maps of the 23 proteins from Table 11 in Progressors (left), and the same structure from the Progressors matrix applied to Control samples (right).

FIG. 43 shows log10 RFU levels for TB Progressors with a Loess fit and 95% bootstrap confidence bounds overlaid on to a control band.

FIG. 44 shows 2-dimensional representation of the directional Mahalanobis Distance model (left panel), as well as bootstrapped AUC for each time bin and the signed Mahalanobis distance per bin (right panel).

FIG. 45 shows cross-validated performance estimates for all top models at each value of k.

FIG. 46 shows signed Mahalanobis Distance (Dₘ) as a function of Time to Treatment for Progressors with 2+ serial samples for the top four most statistically significant models.

FIG. 47 shows performance plots for the TRM5 model. Bootstrapped AUC and signed MD by time bin (left), and ROC by time bin (right).

FIG. 48 shows bootstrapped ROC with operating points emphasizing specificity (left) and sensitivity (right). Shaded boxes represent 95% confidence intervals.

FIG. 49 shows TRM5 sensitivity and specificity as a function of the threshold signed MD decision boundary (left), and the resulting classifications (right).

FIG. 50 shows certain exemplary modified pyrimidines that may be incorporated into aptamers, such as slow off-rate aptamers.

FIG. 51 illustrates a non-limiting exemplary computer system for use with various computer-implemented methods described herein.

FIG. 52 illustrates a non-limiting exemplary aptamer assay that can be used to detect one or more biomarkers in a biological sample.

FIG. 53 shows the validation of the TRM5 model. FIG. 53A shows AUC of all prospective samples in a verification set. FIG. 53B shows TRM5 verification set stratified by time. FIG. 53C shows gates of TB risk in the verification set.

### DETAILED DESCRIPTION

While the invention will be described in conjunction with certain representative embodiments, it will be understood that the invention is defined by the claims, and is not limited to those embodiments.

One skilled in the art will recognize many methods and materials similar or equivalent to those described herein may be used in the practice of the present invention. The present invention is in no way limited to the methods and materials described.

Unless defined otherwise, technical and scientific terms used herein have the meaning commonly understood by one of ordinary skill in the art to which this invention belongs. Although any methods, devices, and materials similar or equivalent to those described herein can be used in the practice of the invention, certain methods, devices, and materials are described herein.

All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include the plural, unless the context clearly dictates otherwise, and may be used interchangeably with “at least one” and “one or more.” Thus, reference to “an aptamer” includes mixtures of aptamers; reference to “a probe” includes mixtures of probes, and the like.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements may include other elements not expressly listed.

The present application includes biomarkers, methods, devices, reagents, systems, and kits for detecting, characterizing, monitoring progression, and/or monitoring treatment of TB infection and/or TB disease.

As used herein, “tuberculosis infection” or “TB infection” refers to the infection of an individual with any of a variety of TB disease-causing mycobacteria (e.g., Mycobacterium tuberculosis). TB infection encompasses both “latent TB infection” (non-transmissible and without symptoms) and “active TB infection” (transmissible and symptomatic). Observable signs of active TB infection include, but are not limited to, chronic cough with blood-tinged sputum, fever, night sweats, and weight loss. As used herein, “individual” and “subject” and “patient” are used interchangeably to refer to a test subject or patient. The individual can be a mammal or a non-mammal. In various embodiments, the individual is a mammal. A mammalian individual can be a human or non-human. In various embodiments, the individual is a human. A “non-infected” individual is one which has not been infected with a TB disease-causing mycobacterium (e.g., Mycobacterium tuberculosis), does not have either latent TB infection or active TB disease, and/or for whom TB infection is not detectable by conventional diagnostic methods.

As used herein, a “subject at risk of TB infection” refers to a subject with or exposed to one or more risk factors for TB infection. Such risk factors include HIV infection, poverty, geographic location, chronic lung disease, poverty, diabetes, genetic susceptibility, imprisonment, etc.

In one aspect, one or more biomarkers are provided for use either alone or in various combinations to detect TB infection and/or disease, to differentiate latent TB infection from active TB disease, to identify subjects at risk of transition from latent to active TB infection, etc. Biomarkers and biomarker panels provided herein are particularly useful for distinguishing samples obtained from individuals with latent TB infection that will advance to active TB disease (or are at high risk of advancing to TB disease) from samples from individuals with latent TB infection that will not advance to active TB disease (or are at low risk of advancing to TB disease).

As described in detail herein, exemplary embodiments include one or more biomarkers selected from the biomarkers in Table 11, including the exemplary 4- and 5-biomarker panels shown in Tables A, B, and C.

Methods and kits are also described herein for grouping the above biomarkers with additional biomarkers described herein and/or with additional biomarkers not listed herein (e.g., biomarkers for diagnosis of TB infection/disease, biomarkers for identification of the strain of infection, biomarkers for identifying antibiotic resistant TB, etc.). In some embodiments, panels of at least two, at least three, at least four, at least five, or at least 6 biomarkers, at least 7 biomarkers, at least 8 biomarkers, at least 9 biomarkers, at least 10 biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, at least 20 biomarkers are provided.

In some embodiments, the number and identity of biomarkers in a panel are selected based on the sensitivity and specificity for the particular combination of biomarker values. The terms “sensitivity” and “specificity” are used herein with respect to the ability to correctly classify an individual, based on one or more biomarker levels detected in a biological sample. “Sensitivity” indicates the performance of the biomarker(s) with respect to correctly classifying individuals as, for example at risk (e.g., high risk or likely) of transitioning from latent TB infection to active TB disease. “Specificity” indicates the performance of the biomarker(s) with respect to correctly classifying individuals who have latent TB infection and are not at risk (e.g., low risk) of transitioning from latent TB infection to active TB disease. For example, 85% specificity and 90% sensitivity for a panel of markers used to test a set of control samples (such as samples from individuals with latent TB infections that did not advance to active TB disease) and test samples (such as samples from TB-infected individuals that developed active TB disease) indicates that 85% of the control samples were correctly classified as control samples by the panel, and 90% of the test samples were correctly classified as test samples by the panel.

In some embodiments, overall performance of a panel of one or more biomarkers is represented by the area-under-the-curve (AUC) value. The AUC value is derived from receiver operating characteristic (ROC) plots, which are exemplified herein. The ROC curve is the plot of the true positive rate (sensitivity) of a test against the false positive rate (1-specificity) of the test. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., low-risk vs. high risk individuals). ROC curves are useful for plotting the performance of a particular feature (e.g., any of the biomarkers described herein and/or any item of additional biomedical information) in distinguishing between two populations (e.g., cases in which subjects transitioned from latent to active TB vs. controls in which TB infection remained latent). Typically, the feature data across the entire population (e.g., all tested subject) are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve.

In some embodiments, methods comprise contacting a sample or a portion of a sample from a subject with at least one capture reagent, wherein each capture reagent specifically binds a biomarker the levels of which are being detected. In some embodiments, the method comprises contacting the sample, or proteins from the sample, with at least one aptamer, wherein each aptamer specifically binds a biomarker, the levels of which are being detected.

In some embodiments, a method comprises detecting the level of at least one biomarker from at least a first panel of biomarkers, the first panel comprising biomarkers selected from the biomarkers in Table 11. In some embodiments, if the level of one or more biomarkers from the first panel are altered relative to a control level, outside a control range, and/or beyond a threshold value, the subject is identified as at-risk of transitioning from latent TB infection to active TB disease. In some embodiments, the subject is identified as having a latent TB infection that is likely to transition into active TB disease if the level of one or more of MMP-1, C9, D-dimer, IP-10, IGFBP-2, MIG, and/or NPS is higher than a control level of the respective biomarker; and/or the levels of one or more of CA2D3, MMP-2, CD79A, PKB a/b/g, PGCB, PABP3, MXRA7, CNTFR alpha, Nr-CAM, Ephrin-A3, CD36 Ag, NDUB4, PCI, BOC, PKB beta, and/or JKIP3 is lower than a control level of the respective biomarker. In some embodiments, methods further comprise detecting at least one biomarker from at least a second panel of biomarkers, the second panel comprising biomarkers for detection of TB infection, detection of active TB disease, characterization of the type, strain, and/or resistance/sensitivity of the TB infection, etc. In some embodiments, if the level of one or more biomarkers from the second panel are altered (e.g., higher or lower) from a control level, outside a control range, and/or beyond a threshold value, the subject and/or the infection are characterized according to the particular second panel being analyzed.

The biomarkers identified herein provide a number of choices for subsets or panels of biomarkers that can be used to effectively characterize TB infection (e.g., characterize the risk of transition from latent to active). Selection of the appropriate number of such biomarkers may depend on the specific combination of biomarkers chosen. In addition, in any of the methods described herein, except where explicitly indicated, a panel of biomarkers may comprise additional biomarkers not listed herein. In some embodiments, a method comprises detecting the levels of a set of biomarkers from Table A, Table B, or Table C in a sample from the subject. In some embodiments, a method comprises detecting the levels of a set of biomarkers from Table C in a sample from the subject. In some embodiments, a method comprises detecting the level of at least one, at least two, at least three, at least four, or five biomarkers selected from C9, IGFBP-2, CD79A, MXRA7, and NR-CAM; or detecting the level of C9, IGFBP-2, CD79A, MXRA7, and NR-CAM.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

Further, in some embodiments, a biological sample may be derived by taking biological samples from a number of individuals and pooling them, or pooling an aliquot of each individual's biological sample. The pooled sample may be treated as described herein for a sample from a single individual, and, for example, if high-risk TB infection is detected in the pooled sample, then each individual biological sample can be re-tested to identify the individual(s) with latent TB infection that is likely to transition into active TB disease within 30 days, 45 days, 60 days, 90 days, 120 days, 180 days, 270 days, 360 days, 450 days, or 540 days.

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” refers to a set of copies of one type or species of molecule or multi-molecular structure. “Target molecules”, “targets”, and “analytes” refer to more than one type or species of molecule or multi-molecular structure. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In some embodiments, a target molecule is a protein, in which case the target molecule may be referred to as a “target protein.”

As used herein, a “capture agent’ or “capture reagent” refers to a molecule that is capable of binding specifically to a biomarker. A “target protein capture reagent” refers to a molecule that is capable of binding specifically to a target protein. Nonlimiting exemplary capture reagents include aptamers, antibodies, adnectins, ankyrins, other antibody mimetics and other protein scaffolds, autoantibodies, chimeras, small molecules, nucleic acids, lectins, ligand-binding receptors, imprinted polymers, avimers, peptidomimetics, hormone receptors, cytokine receptors, synthetic receptors, and modifications and fragments of any of the aforementioned capture reagents. In some embodiments, a capture reagent is selected from an aptamer and an antibody.

The term “antibody” refers to full-length antibodies of any species and fragments and derivatives of such antibodies, including Fab fragments, F(ab′)₂ fragments, single chain antibodies, Fv fragments, and single chain Fv fragments. The term “antibody” also refers to synthetically-derived antibodies, such as phage display-derived antibodies and fragments, affybodies, nanobodies, etc.

As used herein, “marker” and “biomarker” are used interchangeably to refer to a target molecule that indicates or is a sign of a normal or abnormal process in an individual or of a disease or other condition in an individual. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. In some embodiments, a biomarker is a target protein.

As used herein, “biomarker level” and “level” refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “level” depends on the specific design and components of the particular analytical method employed to detect the biomarker.

A “control level” of a target molecule refers to the level of the target molecule in the same sample type from an individual that does not exhibit the characteristic being assayed for (e.g., TB infection, risk of transition from latent TB infection to active TB disease, etc.). A “control level” of a target molecule need not be determined each time the present methods are carried out, and may be a previously determined level that is used as a reference or threshold to determine whether the level in a particular sample is higher or lower than a normal level. In some embodiments, a control level in a method described herein is the level that has been observed in one or more subjects whose latent TB infection did not advance to active TB disease within a particular time period, such as within 540 days or 2 years of sample collection. In some embodiments, a control level in a method described herein is the average or mean level, optionally plus or minus a statistical variation, which has been observed in a plurality of subjects with latent TB infection that did not advance to active TB disease within the particular time period. In some embodiments, a control level in a method described herein is a level that is indicative of chronic latent TB infection.

A “threshold level” of a target molecule refers to the level beyond which (e.g., above or below, depending upon the biomarker) is indicative of or diagnostic for a particular infection, disease, condition, or characteristic thereof. For example, a threshold level of for the likelihood of latent TB infection transitioning into active TB disease is a level of a target molecule beyond which (e.g., above or below, depending upon the biomarker) is indicative of a latnet TB infection that is likely to transition into active TB disease within 30 days, 45 days, 60 days, 90 days, 120 days, 180 days, 270 days, 360 days, 450 days, or 540 days. A “threshold level” of a target molecule need not be determined each time the present methods are carried out, and may be a previously determined level that is used as a reference or threshold to determine whether the level in a particular sample is higher or lower than a normal level. In some embodiments, a subject with a biomarker level beyond (e.g., above or below, depending upon the biomarker) a threshold level has a statistically significant likelihood (e.g., 80% confidence, 85% confidence, 90% confidence, 95% confidence, 98% confidence, 99% confidence, 99.9% confidence, etc.) of having a latent TB infection transition into active TB disease.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (e.g., a diagnosis of the absence of a disease or condition), diagnosed as ill/abnormal (e.g., a diagnosis of the presence, or an assessment of the characteristics, of a disease or condition), and/or high-risk/low-risk (e.g., of developing a disease or condition, of transitioning from a latent infection to an active disease state). The terms “diagnose”, “diagnosing”, “diagnosis”, etc., encompass, with respect to a particular disease or condition: the initial detection of the disease; the characterization or classification of the disease; the characterization of likelihood of advancement of the disease (e.g., from latent to active); the detection of the progression, remission, or recurrence of the disease; and/or the detection of disease response after the administration of a treatment or therapy to the individual.

“Prognose”, “prognosing”, “prognosis”, and variations thereof refer to the prediction of a future course of a disease or condition in an individual who has the disease or condition (e.g., predicting patient survival, predicting likelihood of transition from latent infection to active disease, etc.), and such terms encompass the evaluation of disease response after the administration of a treatment or therapy to the individual.

“Evaluate”, “evaluating”, “evaluation”, and variations thereof encompass both “diagnose” and “prognose” and also encompass determinations or predictions about the future course of a disease or condition in an individual who does not have the disease as well as determinations or predictions regarding the likelihood that a disease or condition will recur in an individual who apparently has been cured of the disease. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “evaluating” TB can include, for example, any of the following: diagnosing a subject with TB infection, diagnosing a subject as suffering from TB disease, determining a subject should undergo further testing (e.g., chest x-ray for TB); prognosing the future course of TB infection/disease in an individual; prognosing a the likelihood of TB transitioning from latent to active; determining whether a TB treatment being administered is effective in the individual; or determining or predicting an individual's response to a TB treatment; or selecting a TB treatment to administer to an individual based upon a determination of the biomarker levels derived from the individual's biological sample.

As used herein, “detecting” or “determining” with respect to a biomarker level includes the use of both the instrument used to observe and record a signal corresponding to a biomarker level and the material/s required to generate that signal. In various embodiments, the level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

As used herein “host biomarkers” are biological molecules (e.g., proteins) that are endogenous to an individual, the expression or level of which is altered (e.g., increased or decreased) upon infection by a pathogenic agent (e.g., Mycobacterium tuberculosis). Detection and/or quantification of host biomarkers allows for characterization of a pathogenic infection.

As used herein “pathogen biomarkers” are molecules (e.g., proteins) that are not endogenous to an infected individual, but produced by a pathogen (e.g., Mycobacterium tuberculosis) that has infected the individual. Detection and/or quantification of pathogen biomarkers (e.g., Mtb biomarkers) allows for characterization of pathogenic infection.

Embodiments described herein include biomarkers, panels of biomarkers, methods, devices, reagents, systems, and kits for detecting, identifying, characterizing, and/or diagnosing infection of a subject (e.g., human subject) with Mycobacterium tuberculosis (Mtb). In particular, embodiments relate to characterizing a latent TB infection: (1) as one is likely to advancing or transition into active TB disease within 30 days, 45 days, 60 days, 90 days, 120 days, 180 days, 270 days, 360 days, 450 days, or 540 days; or (2) as one that is unlikely to advance or transition into active TB disease within 30 days, 45 days, 60 days, 90 days, 120 days, 180 days, 270 days, 360 days, 450 days, or 540 days. Such embodiments involve determining the levels of one or more biomarkers selected from the biomarkers in Table 11; or detecting the levels of a set of biomarkers from Table A, Table B, or Table C; or detecting the levels of a set of biomarkers from Table C; or detecting the level of at least one, at least two, at least three, at least four, or five biomarkers selected from C9, IGFBP-2, CD79A, MXRA7, and NR-CAM; or detecting the level of C9, IGFBP-2, CD79A, MXRA7, and NR-CAM.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore- or cavity-containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, and polymethylpentene. Suitable solid support particles that can be used include, e.g., encoded particles, such as Luminex®-type encoded particles, magnetic particles, and glass particles.

### Exemplary Uses of Biomarkers

In various exemplary embodiments, methods are provided for determining the likelihood or risk of a subject infected with Mycobacterium tuberculosis (e.g., a subject with latent TB infection) transitioning into active TB disease within 30 days, 45 days, 60 days, 90 days, 120 days, 180 days, 270 days, 360 days, 450 days, or 540 days.

In some embodiments, a finding that a TB-infected subject is unlikely to transition into active TB disease indicates that the subject is not presently at significant risk of active TB disease.

In certain exemplary embodiments, methods are provided for determining the likelihood or risk that a non-infected subject would transition from latent infection to active TB disease within 30 days, 45 days, 60 days, 90 days, 120 days, 180 days, 270 days, 360 days, 450 days, or 540 days, should they become infected by Mycobacterium tuberculosis (or another agent causative of TB).

In some embodiments, methods comprise testing a subject for TB infection, for example, by skin test, sputum culture, blood test, tissue culture, body fluid culture, chest x-ray, and/or using the methods described in U.S. Prov. Pat. App. 61/987,888, which is herein incorporated by reference in its entirety. Following a determination that a subject is infected with TB (e.g. latent infection), and a determination (e.g., by monitoring symptoms, by chest x-ray, etc.) that a subject does not have active TB disease, methods described herein are employed to determine the likelihood that such an infection may progress into active TB disease.

In addition to testing biomarker levels (e.g., one or more of the TB biomarkers identified in experiments conducted during development of embodiments of the present invention (e.g., one or more biomarkers selected from the biomarkers in Table 11; or a set of biomarkers from Table A, Table B, or Table C; or a set of biomarkers from Table C; or at least one, at least two, at least three, at least four, or five biomarkers selected from C9, IGFBP-2, CD79A, MXRA7, and NR-CAM; or C9, IGFBP-2, CD79A, MXRA7, and NR-CAM) as a stand-alone diagnostic test, in some embodiments, biomarker levels are tested in conjunction with other markers or assays for characterizing TB (e.g., skin test, sputum culture, blood test, tissue culture, body fluid culture, chest x-ray, methods described in U.S. Prov. Pat. App. 61/987,888 (herein incorporated by reference in its entirety), etc.). In addition to testing biomarker levels in conjunction with other TB diagnostic methods, information regarding the biomarkers can also be evaluated in conjunction with other types of data, particularly data that indicates an individual's risk for TB (e.g., lifestyle, location, age, etc.). These various data can be assessed by automated methods, such as a computer program/software, which can be embodied in a computer or other apparatus/device.

### Detection and Determination of Biomarkers and Biomarker Levels

A biomarker level for the biomarkers described herein can be detected using any of a variety of known analytical methods. In one embodiment, a biomarker level is detected using a capture reagent. In various embodiments, the capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to aptamers, antibodies, adnectins, ankyrins, other antibody mimetics and other protein scaffolds, autoantibodies, chimeras, small molecules, F(ab′)₂ fragments, single chain antibody fragments, Fv fragments, single chain Fv fragments, nucleic acids, lectins, ligand-binding receptors, affybodies, nanobodies, imprinted polymers, avimers, peptidomimetics, hormone receptors, cytokine receptors, and synthetic receptors, and modifications and fragments of these.

In some embodiments, biomarker presence or level is detected using a biomarker/capture reagent complex.

In some embodiments, the biomarker presence or level is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, biomarker presence or level is detected directly from the biomarker in a biological sample.

In some embodiments, biomarkers are detected using a multiplexed format that allows for the simultaneous detection of two or more biomarkers in a biological sample. In some embodiments of the multiplexed format, capture reagents are immobilized, directly or indirectly, covalently or non-covalently, in discrete locations on a solid support. In some embodiments, a multiplexed format uses discrete solid supports where each solid support has a unique capture reagent associated with that solid support, such as, for example quantum dots. In some embodiments, an individual device is used for the detection of each one of multiple biomarkers to be detected in a biological sample. Individual devices can be configured to permit each biomarker in the biological sample to be processed simultaneously. For example, a microtiter plate can be used such that each well in the plate is used to analyze one or more of multiple biomarkers to be detected in a biological sample.

In one or more of the foregoing embodiments, a fluorescent tag can be used to label a component of the biomarker/capture reagent complex to enable the detection of the biomarker level. In various embodiments, the fluorescent label can be conjugated to a capture reagent specific to any of the biomarkers described herein using known techniques, and the fluorescent label can then be used to detect the corresponding biomarker level. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In some embodiments, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In some embodiments, the dye molecule includes a first type and a second type of dye molecule, such as, e.g., two different AlexaFluor molecules. In some embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more embodiments, a chemiluminescence tag can optionally be used to label a component of the biomarker/capture complex to enable the detection of a biomarker level. Suitable chemiluminescent materials include any of oxalyl chloride, Rodamin 6G, Ru(bipy)32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In some embodiments, the detection method includes an enzyme/substrate combination that generates a detectable signal that corresponds to the biomarker level. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In some embodiments, the detection method can be a combination of fluorescence, chemiluminescence, radionuclide or enzyme/substrate combinations that generate a measurable signal. In some embodiments, multimodal signaling could have unique and advantageous characteristics in biomarker assay formats.

In some embodiments, the biomarker levels for the biomarkers described herein can be detected using any analytical methods including, singleplex aptamer assays, multiplexed aptamer assays, singleplex or multiplexed immunoassays, mRNA expression profiling, miRNA expression profiling, mass spectrometric analysis, histological/cytological methods, etc. as discussed below.

### Determination of Biomarker Levels using Aptamer-Based Assays

Assays directed to the detection and quantification of physiologically significant molecules in biological samples and other samples are important tools in scientific research and in the health care field. One class of such assays involves the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. No. 6,242,246, U.S. Pat. No. 6,458,543, and U.S. Pat. No. 6,503,715, each of which is entitled “Nucleic Acid Ligand Diagnostic Biochip”. Once the microarray is contacted with a sample, the aptamers bind to their respective target molecules present in the sample and thereby enable a determination of a biomarker level corresponding to a biomarker.

As used herein, an “aptamer” refers to a nucleic acid that has a specific binding affinity for a target molecule. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that has a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Any of the aptamer methods disclosed herein can include the use of two or more aptamers that specifically bind the same target molecule. As further described below, an aptamer may include a tag. If an aptamer includes a tag, all copies of the aptamer need not have the same tag. Moreover, if different aptamers each include a tag, these different aptamers can have either the same tag or a different tag.

An aptamer can be identified using any known method, including the SELEX process. Once identified, an aptamer can be prepared or synthesized in accordance with any known method, including chemical synthetic methods and enzymatic synthetic methods.

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH₂), 2′-fluoro (2′-F), and/or 2′-O-methyl (2′-OMe). See also, U.S. Patent Application Publication No. 2009/0098549, entitled “SELEX and PHOTOSELEX”, which describes nucleic acid libraries having expanded physical and chemical properties and their use in SELEX and photoSELEX.

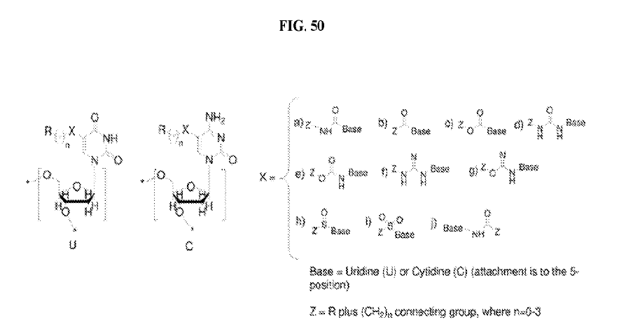

SELEX can also be used to identify aptamers that have desirable off-rate characteristics. See U.S. Publication No. US 2009/0004667, entitled “Method for Generating Aptamers with Improved Off-Rates”, which describes improved SELEX methods for generating aptamers that can bind to target molecules. Methods for producing aptamers and photoaptamers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers with improved off-rate performance. Nonlimiting exemplary modified nucleotides include, for example, the modified pyrimidines shown in FIG. 50. In some embodiments, an aptamer comprises at least one nucleotide with a modification, such as a base modification. In some embodiments, an aptamer comprises at least one nucleotide with a hydrophobic modification, such as a hydrophobic base modification, allowing for hydrophobic contacts with a target protein. Such hydrophobic contacts, in some embodiments, contribute to greater affinity and/or slower off-rate binding by the aptamer. Nonlimiting exemplary nucleotides with hydrophobic modifications are shown in FIG. 50. In some embodiments, an aptamer comprises at least two, at least three, at least four, at least five, at least six, at least seven, at least eight, at least nine, or at least 10 nucleotides with hydrophobic modifications, where each hydrophobic modification may be the same or different from the others. In some embodiments, at least one, at least two, at least three, at least four, at least five, at least six, at least seven, at least eight, at least nine, or at least 10 hydrophobic modifications in an aptamer may be independently selected from the hydrophobic modifications shown in FIG. 50.

In some embodiments, a slow off-rate aptamer (including an aptamers comprising at least one nucleotide with a hydrophobic modification) has an off-rate (t½) of ≥30 minutes, ≥60 minutes, ≥90 minutes, ≥120 minutes, ≥150 minutes, ≥180 minutes, ≥210 minutes, or ≥240 minutes.

As used herein, a “SOMAmer” or “Slow Off-Rate Aptamer” refers to an aptamer having improved off-rate characteristics. Slow off-rate aptamers can be generated using the modified SELEX methods described in U.S. Publication No. 20090004667; herein incorporated by reference in its entirety. The methods disclosed herein are in no way limited to slow off-rate aptamers, however, use of the slow off-rate process described in U.S. Pat. No. 7,964,356 and U.S. Publication No. 2012/0115752 (herein incorporated by reference in their entireties), may provide improved results.

In some embodiments, an assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. No. 5,763,177, U.S. Pat. No. 6,001,577, and U.S. Pat. No. 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker level corresponding to a biomarker in the test sample.

In some assay formats, the aptamers are immobilized on the solid support prior to being contacted with the sample. Under certain circumstances, however, immobilization of the aptamers prior to contact with the sample may not provide an optimal assay. For example, pre-immobilization of the aptamers may result in inefficient mixing of the aptamers with the target molecules on the surface of the solid support, perhaps leading to lengthy reaction times and, therefore, extended incubation periods to permit efficient binding of the aptamers to their target molecules. Further, when photoaptamers are employed in the assay and depending upon the material utilized as a solid support, the solid support may tend to scatter or absorb the light used to effect the formation of covalent bonds between the photoaptamers and their target molecules. Moreover, depending upon the method employed, detection of target molecules bound to their aptamers can be subject to imprecision, since the surface of the solid support may also be exposed to and affected by any labeling agents that are used. Finally, immobilization of the aptamers on the solid support generally involves an aptamer-preparation step (i.e., the immobilization) prior to exposure of the aptamers to the sample, and this preparation step may affect the activity or functionality of the aptamers.

Aptamer assays that permit an aptamer to capture its target in solution and then employ separation steps that are designed to remove specific components of the aptamer-target mixture prior to detection have also been described (see U.S. Publication No. 2009/0042206, entitled “Multiplexed Analyses of Test Samples”). The described aptamer assay methods enable the detection and quantification of a non-nucleic acid target (e.g., a protein target) in a test sample by detecting and quantifying a nucleic acid (i.e., an aptamer). The described methods create a nucleic acid surrogate (i.e., the aptamer) for detecting and quantifying a non-nucleic acid target, thus allowing the wide variety of nucleic acid technologies, including amplification, to be applied to a broader range of desired targets, including protein targets.

Aptamers can be constructed to facilitate the separation of the assay components from an aptamer biomarker complex (or photoaptamer biomarker covalent complex) and permit isolation of the aptamer for detection and/or quantification. In one embodiment, these constructs can include a cleavable or releasable element within the aptamer sequence. In other embodiments, additional functionality can be introduced into the aptamer, for example, a labeled or detectable component, a spacer component, or a specific binding tag or immobilization element. For example, the aptamer can include a tag connected to the aptamer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to an aptamer, thereby allowing for the release of the aptamer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target. In some embodiments of the methods described herein, the molecular capture reagents comprise an aptamer or an antibody or the like and the specific target may be a biomarker described herein (e.g., the biomarkers in Table 11).

In some embodiments, a method for signal generation takes advantage of anisotropy signal change due to the interaction of a fluorophore-labeled capture reagent with its specific biomarker target. When the labeled capture reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based aptamer assay that can be used to detect a biomarker level in a biological sample includes the following: (a) preparing a mixture by contacting the biological sample with an aptamer that includes a first tag and has a specific affinity for the biomarker, wherein an aptamer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the aptamer affinity complex; (e) releasing the aptamer affinity complex from the first solid support; (f) exposing the released aptamer affinity complex to a second solid support that includes a second capture element and allowing the second tag to associate with the second capture element; (g) removing any non-complexed aptamer from the mixture by partitioning the non-complexed aptamer from the aptamer affinity complex; (h) eluting the aptamer from the solid support; and (i) detecting the biomarker by detecting the aptamer component of the aptamer affinity complex.

A non-limiting exemplary method of detecting biomarkers in a biological sample using aptamers is described, for example, in Kraemer et al., 2011, PLoS One 6(10): e26332; herein incorporated by reference in its entirety.

### Determination of Biomarker Levels using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies and fragments thereof are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or level corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (1125) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or for quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 386 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Determination of Biomarker Levels using Gene Expression Profiling

Measuring mRNA in a biological sample may, in some embodiments, be used as a surrogate for detection of the level of the corresponding protein in the biological sample. Thus, in some embodiments, a biomarker or biomarker panel described herein can be detected by detecting the appropriate RNA.

In some embodiments, mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

### Detection of Biomarkers Using In Vivo Molecular Imaging Technologies

In some embodiments, a biomarker described herein may be used in molecular imaging tests. For example, an imaging agent can be coupled to a capture reagent, which can be used to detect the biomarker in vivo.

In vivo imaging technologies provide non-invasive methods for determining the state of a particular disease in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the biomarker in vivo.

The use of in vivo molecular imaging technologies is expanding due to various advances in technology. These advances include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, such as an aptamer or an antibody, for example, and/or with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, computed tomography scanning, positron emission tomography (PET), single photon emission computed tomography (SPECT), and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Similarly, aptamers may be used for such in vivo imaging diagnostic methods. For example, an aptamer that was used to identify a particular biomarker described herein may be appropriately labeled and injected into an individual to detect the biomarker in vivo. The label used will be selected in accordance with the imaging modality to be used, as previously described. Aptamer-directed imaging agents could have unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

Other techniques are review, for example, in N. Blow, Nature Methods, 6, 465-469, 2009; herein incorporated by reference in its entirety.

### Determination of Biomarkers using Histology/Cytology Methods

In some embodiments, the biomarkers described herein may be detected in a variety of tissue samples using histological or cytological methods. For example, endo- and trans-bronchial biopsies, fine needle aspirates, cutting needles, and core biopsies can be used for histology. Bronchial washing and brushing, pleural aspiration, and sputum, can be used for cyotology. Any of the biomarkers identified herein can be used to stain a specimen as an indication of disease.

In some embodiments, one or more capture reagent/s specific to the corresponding biomarker/s are used in a cytological evaluation of a sample and may include one or more of the following: collecting a cell sample, fixing the cell sample, dehydrating, clearing, immobilizing the cell sample on a microscope slide, permeabilizing the cell sample, treating for analyte retrieval, staining, destaining, washing, blocking, and reacting with one or more capture reagent/s in a buffered solution. In another embodiment, the cell sample is produced from a cell block.

In some embodiments, one or more capture reagent/s specific to the corresponding biomarkers are used in a histological evaluation of a tissue sample and may include one or more of the following: collecting a tissue specimen, fixing the tissue sample, dehydrating, clearing, immobilizing the tissue sample on a microscope slide, permeabilizing the tissue sample, treating for analyte retrieval, staining, destaining, washing, blocking, rehydrating, and reacting with capture reagent/s in a buffered solution. In another embodiment, fixing and dehydrating are replaced with freezing.

In another embodiment, the one or more aptamer/s specific to the corresponding biomarker/s are reacted with the histological or cytological sample and can serve as the nucleic acid target in a nucleic acid amplification method. Suitable nucleic acid amplification methods include, for example, PCR, q-beta replicase, rolling circle amplification, strand displacement, helicase dependent amplification, loop mediated isothermal amplification, ligase chain reaction, and restriction and circularization aided rolling circle amplification.

In one embodiment, the one or more capture reagent/s specific to the corresponding biomarkers for use in the histological or cytological evaluation are mixed in a buffered solution that can include any of the following: blocking materials, competitors, detergents, stabilizers, carrier nucleic acid, polyanionic materials, etc.

A “cytology protocol” generally includes sample collection, sample fixation, sample immobilization, and staining. “Cell preparation” can include several processing steps after sample collection, including the use of one or more aptamers for the staining of the prepared cells.

### Determination of Biomarker Levels using Mass Spectrometry Methods

A variety of configurations of mass spectrometers can be used to detect biomarker levels. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker levels can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)N, atmospheric pressure photoionization mass spectrometry (APPI-MS), APPI-MS/MS, and APPI-(MS)N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker levels. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

The foregoing assays enable the detection of biomarker levels that are useful in the methods described herein, where the methods comprise detecting, in a biological sample from an individual, at least one, at least two, at least three, at least four, at least five, at least six, at least seven, at least eight, or at least nine biomarkers selected from the described herein. Thus, while some of the described biomarkers may be useful alone for detecting TB infection, methods are also described herein for the grouping of multiple biomarkers and subsets of the biomarkers to form panels of two or more biomarkers. In accordance with any of the methods described herein, biomarker levels can be detected and classified individually or they can be detected and classified collectively, as for example in a multiplex assay format.

### Classification of Biomarkers and Calculation of Disease Scores

In some embodiments, a biomarker “signature” for a given diagnostic test contains a set of markers, each marker having characteristic levels in the populations of interest. Characteristic levels, in some embodiments, may refer to the mean or average of the biomarker levels for the individuals in a particular group. In some embodiments, a diagnostic method described herein can be used to assign an unknown sample from an individual into one of two groups: TB infected or non-infected, active TB or no active TB, latent TB or no TB infection, etc. The assignment of a sample into one of two or more groups (e.g., TB infection, latent infection, active infection, non-infected, etc.) is known as classification, and the procedure used to accomplish this assignment is known as a classifier or a classification method. Classification methods may also be referred to as scoring methods. There are many classification methods that can be used to construct a diagnostic classifier from a set of biomarker levels. In some instances, classification methods are performed using supervised learning techniques in which a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

Common approaches for developing diagnostic classifiers include decision trees; bagging+boosting+forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009.

To produce a classifier using supervised learning techniques, a set of samples called training data are obtained. In the context of diagnostic tests, training data includes samples from the distinct groups (classes) to which unknown samples will later be assigned. For example, samples collected from individuals in a control population and individuals in a particular disease population can constitute training data to develop a classifier that can classify unknown samples (or, more particularly, the individuals from whom the samples were obtained) as either having the disease or being free from the disease. The development of the classifier from the training data is known as training the classifier. Specific details on classifier training depend on the nature of the supervised learning technique. Training a naïve Bayesian classifier is an example of such a supervised learning technique (see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009). Training of a naive Bayesian classifier is described, e.g., in U.S. Publication Nos: 2012/0101002 and 2012/0077695.

Since typically there are many more potential biomarker levels than samples in a training set, care must be used to avoid over-fitting. Over-fitting occurs when a statistical model describes random error or noise instead of the underlying relationship. Over-fitting can be avoided in a variety of way, including, for example, by limiting the number of markers used in developing the classifier, by assuming that the marker responses are independent of one another, by limiting the complexity of the underlying statistical model employed, and by ensuring that the underlying statistical model conforms to the data.

An illustrative example of the development of a diagnostic test using a set of biomarkers includes the application of a naive Bayes classifier, a simple probabilistic classifier based on Bayes theorem with strict independent treatment of the biomarkers. Each biomarker is described by a class-dependent probability density function (PDF) for the measured RFU values or log RFU (relative fluorescence units) values in each class. The joint PDFs for the set of markers in one class is assumed to be the product of the individual class-dependent PDFs for each biomarker. Training a naïve Bayes classifier in this context amounts to assigning parameters (“parameterization”) to characterize the class dependent PDFs. Any underlying model for the class-dependent PDFs may be used, but the model should generally conform to the data observed in the training set.

The performance of the naive Bayes classifier is dependent upon the number and quality of the biomarkers used to construct and train the classifier. A single biomarker will perform in accordance with its KS-distance (Kolmogorov-Smirnov). The addition of subsequent markers with good KS distances (>0.3, for example) will, in general, improve the classification performance if the subsequently added markers are independent of the first marker. Using the sensitivity plus specificity as a classifier score, many high scoring classifiers can be generated with a variation of a greedy algorithm. (A greedy algorithm is any algorithm that follows the problem solving metaheuristic of making the locally optimal choice at each stage with the hope of finding the global optimum.)

Another way to depict classifier performance is through a receiver operating characteristic (ROC), or simply ROC curve or ROC plot. The ROC is a graphical plot of the sensitivity, or true positive rate, vs. false positive rate (1—specificity or 1—true negative rate), for a binary classifier system as its discrimination threshold is varied. The ROC can also be represented equivalently by plotting the fraction of true positives out of the positives (TPR=true positive rate) vs. the fraction of false positives out of the negatives (FPR=false positive rate). Also known as a Relative Operating Characteristic curve, because it is a comparison of two operating characteristics (TPR & FPR) as the criterion changes. The area under the ROC curve (AUC) is commonly used as a summary measure of diagnostic accuracy. It can take values from 0.0 to 1.0. The AUC has an important statistical property: the AUC of a classifier is equivalent to the probability that the classifier will rank a randomly chosen positive instance higher than a randomly chosen negative instance (Fawcett T, 2006. An introduction to ROC analysis. Pattern Recognition Letters. 27: 861-874). This is equivalent to the Wilcoxon test of ranks (Hanley, J. A., McNeil, B. J., 1982. The meaning and use of the area under a receiver operating characteristic (ROC) curve. Radiology 143, 29-36.).

Exemplary embodiments use any number of the biomarkers provided herein in various combinations to produce diagnostic tests for detecting TB infection in a sample from an individual. The markers provided herein can be combined in many ways to produce classifiers. For example, a classifier may comprise one or more biomarkers selected from the biomarkers in Table 11; or a set of biomarkers from Table A, Table B, or Table C; or a set of biomarkers from Table C; or at least one, at least two, at least three, at least four, or five biomarkers selected from C9, IGFBP-2, CD79A, MXRA7, and NR-CAM; or C9, IGFBP-2, CD79A, MXRA7, and NR-CAM; or any suitable combinations or sub-combinations thereof.

In some embodiments, once a panel is defined to include a particular set of biomarkers and a classifier is constructed from a set of training data, the diagnostic test parameters are complete. In some embodiments, a biological sample is run in one or more assays to produce the relevant quantitative biomarker levels used for classification. The measured biomarker levels are used as input for the classification method that outputs a classification and an optional score for the sample that reflects the confidence of the class assignment.

In some embodiments, a biological sample is optionally diluted and run in a multiplexed aptamer assay, and data is assessed as follows. First, the data from the assay are optionally normalized and calibrated, and the resulting biomarker levels are used as input to a Bayes classification scheme. Second, the log-likelihood ratio is computed for each measured biomarker individually and then summed to produce a final classification score, which is also referred to as a diagnostic score. The resulting assignment as well as the overall classification score can be reported. In some embodiments, the individual log-likelihood risk factors computed for each biomarker level can be reported as well.

### Kits

Any combination of the biomarkers described herein can be detected using a suitable kit, such as for use in performing the methods disclosed herein. The biomarkers described herein may be combined in any suitable combination, or may be combined with other markers not described herein. Furthermore, any kit can contain one or more detectable labels as described herein, such as a fluorescent moiety, etc.

In some embodiments, a kit includes (a) one or more capture reagents (such as, for example, at least one aptamer or antibody) for detecting one or more biomarkers in a biological sample, and optionally (b) one or more software or computer program products for predicting whether the individual from whom the biological sample was obtained is TB infected. Alternatively, rather than one or more computer program products, one or more instructions for manually performing the above steps by a human can be provided.

In some embodiments, a kit comprises a solid support, a capture reagent, and a signal generating material. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, antibody capture agents, positive control samples, negative control samples, software and information such as protocols, guidance and reference data.

In some embodiments, kits are provided for the analysis of TB infection, wherein the kits comprise PCR primers for one or more biomarkers described herein. In some embodiments, a kit may further include instructions for use and correlation of the biomarkers with TB infection. In some embodiments, a kit may include a DNA array containing the complement of one or more of the biomarkers described herein, reagents, and/or enzymes for amplifying or isolating sample DNA. The kits may include reagents for real-time PCR, for example, TaqMan probes and/or primers, and enzymes.

For example, a kit can comprise (a) reagents comprising at least one capture reagent for determining the level of one or more biomarkers in a test sample, and optionally (b) one or more algorithms or computer programs for performing the steps of comparing the amount of each biomarker quantified in the test sample to one or more predetermined cutoffs. In some embodiments, an algorithm or computer program assigns a score for each biomarker quantified based on said comparison and, in some embodiments, combines the assigned scores for each biomarker quantified to obtain a total score. Further, in some embodiments, an algorithm or computer program compares the total score with a predetermined score, and uses the comparison to determine, for example, likelihood of latent TB infection advancing into active TB disease. Alternatively, rather than one or more algorithms or computer programs, one or more instructions for manually performing the above steps by a human can be provided.

### Computer Methods and Software

Once a biomarker or biomarker panel is selected, a method may comprise the following: 1) collect or otherwise obtain a biological sample; 2) perform an analytical method to detect and measure the biomarker or biomarkers in the panel in the biological sample; and 3) report the results of the biomarker levels. In some embodiments, the results of the biomarker levels are reported qualitatively rather than quantitatively, such as, for example, a proposed diagnosis or numeric result indicating the percent likelihood (e.g., within a margin of error) of a latent infection transitioning to active TB. In some embodiments, a qualitative or quantitative risk of developing active TB disease within a particular time period is provided (e.g., within 30 days, 45 days, 60 days, 90 days, 120 days, 180 days, 270 days, 360 days, 450 days, or 540 days). In some embodiments, a method comprises the following: 1) collect or otherwise obtain a biological sample; 2) perform an analytical method to detect and measure the biomarker or biomarkers in the panel in the biological sample; 3) perform any data normalization or standardization; 4) calculate each biomarker level; and 5) report the results of the biomarker levels. In some embodiments, the biomarker levels are combined in some way and a single value for the combined biomarker levels is reported. In this approach, in some embodiments, the reported value may be a single number determined from the sum of all the marker calculations that is compared to a pre-set threshold value that is an indication of the presence or absence of disease. Or the diagnostic score may be a series of bars that each represent a biomarker value and the pattern of the responses may be compared to a pre-set pattern for determination of the presence or absence of disease.

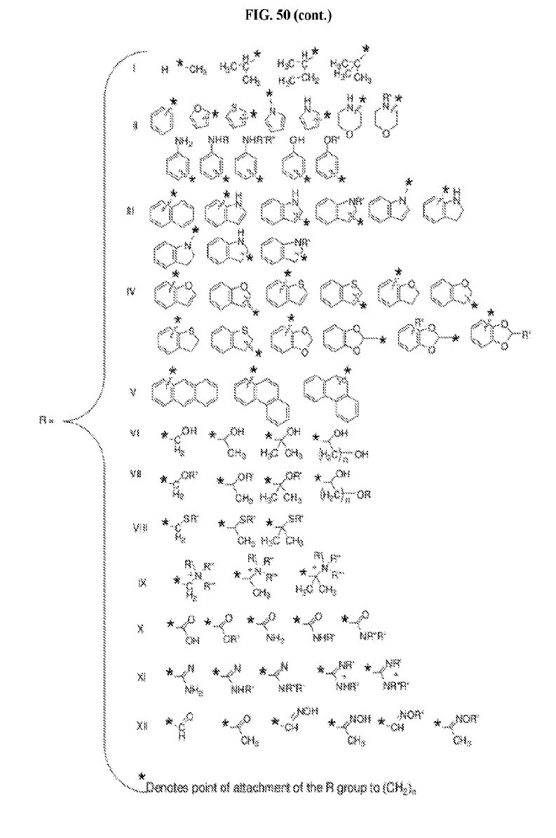

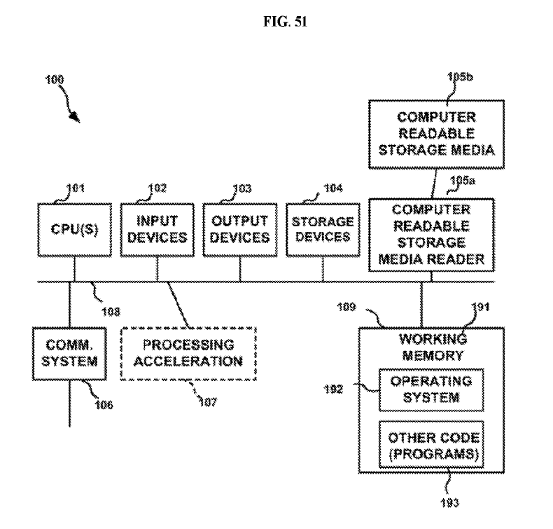

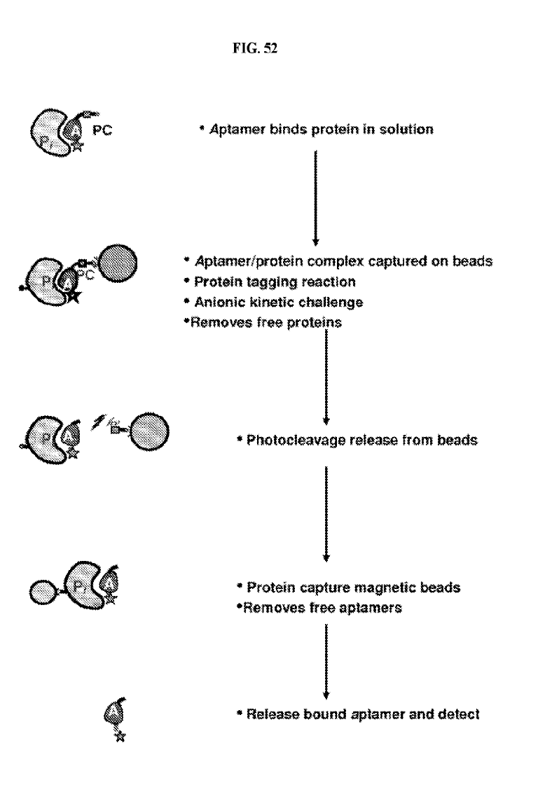

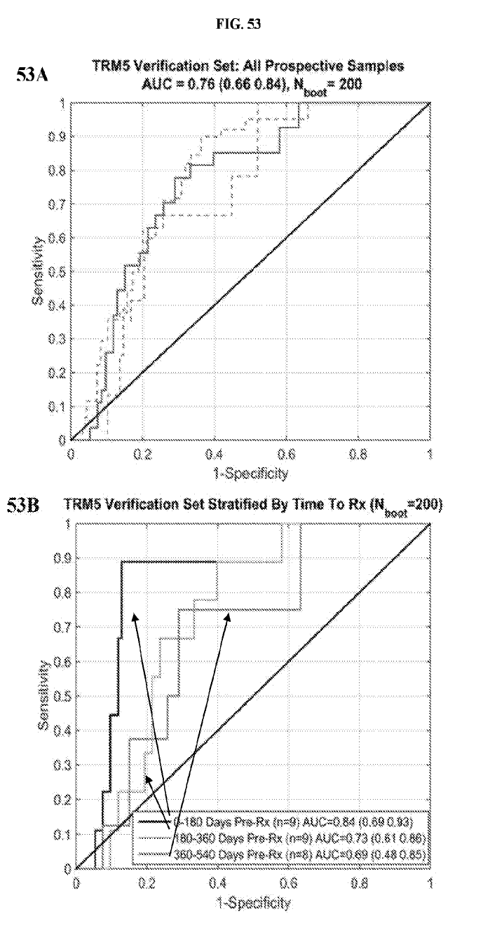

At least some embodiments of the methods described herein can be implemented with the use of a computer. An example of a computer system 100 is shown in FIG. 51. With reference to FIG. 51, system 100 is shown comprised of hardware elements that are electrically coupled via bus 108, including a processor 101, input device 102, output device 103, storage device 104, computer-readable storage media reader 105a, communications system 106 processing acceleration (e.g., DSP or special-purpose processors) 107 and memory 109. Computer-readable storage media reader 105a is further coupled to computer-readable storage media 105b, the combination comprehensively representing remote, local, fixed and/or removable storage devices plus storage media, memory, etc. for temporarily and/or more permanently containing computer-readable information, which can include storage device 104, memory 109 and/or any other such accessible system 100 resource. System 100 also comprises software elements (shown as being currently located within working memory 191) including an operating system 192 and other code 193, such as programs, data and the like.

With respect to FIG. 51, system 100 has extensive flexibility and configurability. Thus, for example, a single architecture might be utilized to implement one or more servers that can be further configured in accordance with currently desirable protocols, protocol variations, extensions, etc. However, it will be apparent to those skilled in the art that embodiments may well be utilized in accordance with more specific application requirements. For example, one or more system elements might be implemented as sub-elements within a system 100 component (e.g., within communications system 106). Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system can comprise a database containing features of biomarkers characteristic of TB infection. The biomarker data (or biomarker information) can be utilized as an input to the computer for use as part of a computer implemented method. The biomarker data can include the data as described herein.

In one aspect, the system further comprises one or more devices for providing input data to the one or more processors.

In some embodiments, the system further comprises a memory for storing a data set of ranked data elements.

In another aspect, the device for providing input data comprises a detector for detecting the characteristic of the data element, e.g., such as a mass spectrometer or gene chip reader.

The system additionally may comprise a database management system. User requests or queries can be formatted in an appropriate language understood by the database management system that processes the query to extract the relevant information from the database of training sets.

The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests.

The system may include an operating system (e.g., UNIX® or Linux) for executing instructions from a database management system. In one aspect, the operating system can operate on a global communications network, such as the internet, and utilize a global communications network server to connect to such a network.

The system may include one or more devices that comprise a graphical display interface comprising interface elements such as buttons, pull down menus, scroll bars, fields for entering text, and the like as are routinely found in graphical user interfaces known in the art. Requests entered on a user interface can be transmitted to an application program in the system for formatting to search for relevant information in one or more of the system databases. Requests or queries entered by a user may be constructed in any suitable database language.

The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

The system can be in communication with an input device for providing data regarding data elements to the system (e.g., expression values). In one aspect, the input device can include a gene expression profiling system including, e.g., a mass spectrometer, gene chip or array reader, and the like.

The methods and apparatus for analyzing biomarker information according to various embodiments may be implemented in any suitable manner, for example, using a computer program operating on a computer system. A conventional computer system comprising a processor and a random access memory, such as a remotely-accessible application server, network server, personal computer or workstation may be used. Additional computer system components may include memory devices or information storage systems, such as a mass storage system and a user interface, for example a conventional monitor, keyboard and tracking device. The computer system may be a stand-alone system or part of a network of computers including a server and one or more databases.

The biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the biomarkers. The computer program may comprise multiple modules performing various functions or operations, such as a processing module for processing raw data and generating supplemental data and an analysis module for analyzing raw data and supplemental data to generate a disease status and/or diagnosis. Methods may comprise generating or collecting any other information, including additional biomedical information, regarding the condition of the individual relative to the disease, identifying whether further tests may be desirable, or otherwise evaluating the health status of the individual.

Some embodiments described herein can be implemented so as to include a computer program product. A computer program product may include a computer readable medium having computer readable program code embodied in the medium for causing an application program to execute on a computer with a database.

As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one aspect, a computer program product is provided for characterizing the TB-infection status (e.g., likelihood of advancement to active TB) of a subject. The computer program product includes a computer readable medium embodying program code executable by a processor of a computing device or system, the program code comprising: code that retrieves data attributed to a biological sample from an individual, wherein the data comprises biomarker levels that correspond to one or more of the biomarkers described herein, and code that executes a classification method that indicates the TB-infection status of the individual as a function of the biomarker levels.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description. Therefore, it is desired that embodiments also be considered protected by this patent in their program code means as well. Furthermore, the embodiments may be embodied as code stored in a computer-readable memory of virtually any kind including, without limitation, RAM, ROM, magnetic media, optical media, or magneto-optical media. Even more generally, the embodiments could be implemented in software, or in hardware, or any combination thereof including, but not limited to, software running on a general purpose processor, microcode, programmable logic arrays (PLAs), or application-specific integrated circuits (ASICs).

It is also envisioned that embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

### Methods of Treatment

In some embodiments, following characterization of a subject's TB status (e.g., no infection; latent infection not likely to advance to active TB; latent infection—likely to advance to active TB within 30 days, 45 days, 60 days, 90 days, 120 days, 180 days, 270 days, 360 days, 450 days, or 540 days; active TB disease; etc.), the subject is treated for TB infection. In some embodiments, medications used to treat latent TB infection include: isoniazid (INH), rifampin (RIF), and rifapentine (RPT). In some embodiments, TB disease is treated by taking several drugs for 6 to 9 months. There are 10 drugs currently approved by the U.S. Food and Drug Administration (FDA) for treating TB. Of the approved drugs, the first-line anti-TB agents that form the core of treatment regimens include: isoniazid (INH), rifampin (RIF), ethambutol (EMB), and pyrazinamide (PZA). Regimens for treating TB disease have an initial phase of 2 months, followed by a choice of several options for the continuation phase of either 4 or 7 months (total of 6 to 9 months for treatment).

In some embodiments, methods of monitoring TB infection/disease and/or treatment of TB infection/disease are provided. In some embodiments, the present methods of detecting TB infection are carried out at a time 0. In some embodiments, the method is carried out again at a time 1, and optionally, a time 2, and optionally, a time 3, etc., in order to monitor the progression of TB infection or to monitor the effectiveness of one or more treatments of TB. Time points for detection may be separated by, for example at least 1 day, at least 2 days, at least 4 days, at least 1 week, at least 2 weeks, at least 1 month, at least 2 months, at least 3 months, at least 4 months, at least 6 months, or by 1 year or more. In some embodiments, a treatment regimen is altered based upon the results of monitoring (e.g., upon determining that a first treatment is ineffective).

### EXAMPLES

### Example 1

### Samples and Subjects

### Normalization and Calibration

Samples were obtained from a study of TB risk conducted by the South African Tuberculosis Vaccine Initiative (SATVI) in collaboration with the University of Cape Town (UCT). The TB Risk study enrolled 6,363 adolescents (12-18 years of age) prospectively at several high schools in an area ˜100 km from Cape Town with a high burden of TB. Blood was collected at mobile collection centers from participants at 6 month intervals between 2006 and 2008 and during this time some participants developed active TB. TB diagnosis was determined by bacteriological testing, though subjects had positive Quantiferon Gold In-Tube (QFT) and tuberculin skin tests (TST) at time of enrollment as immunological evidence of Mtb infection. The samples used for the SOMAscan V3+ 3000plex were CPT heparin plasma. TB case samples were placed into ‘bins’ along with matched control samples based on gender, age, ethnicity, high school and history of TB. A tabulation of the resulting patient demographics is summarized in Table 1 and Table 2 below.

TABLE 1
Participant demographics for the case cohort in discovery (training) and verification
(test) sets, as well as a p-value for the two group comparison using a t-test

| | | Age | Male | Black | Coloured | |
|---|---|---|---|---|---|---|
| | Participants | Mean, (min-max) | n, (%) | n, (%) | n, (%) | Prior TB |
| Discovery | 29 | 15.79 (12-18) | 9 (31%) | 2 (7%) | 27 (93%) | 3 (10%) |
| Verification | 15 | 15.13 (12-18) | 4 (28%) | 1 (7%) | 14 (93%) | 2 (7%) |
| p | | 0.3272 | 1 | 1 | 1 | 1 |

TABLE 2
Number of study participants, samples, and sample locations
for the TB case and non-TB control cohorts.

| | Participants | | Samples | | | |
|---|---|---|---|---|---|---|
| | Discovery | Verification | Discovery | Verification | SATVI | Seattle |
| Cases | 29 | 15 | 84 | 40 | 24* | 60* |
| Controls | 68 | 38 | 199 | 93 | 97* | 102* |

*discovery samples only listed (verification samples are blinded)

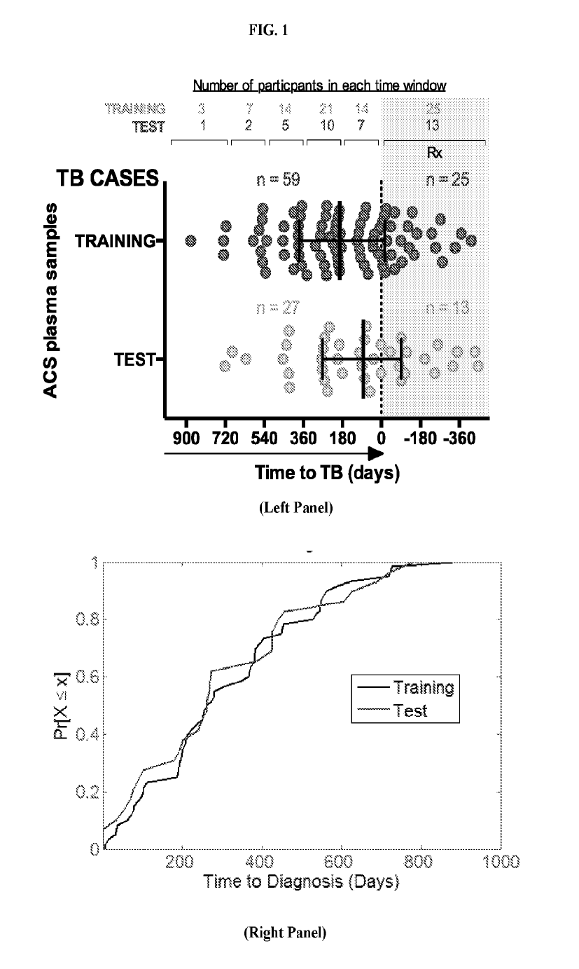

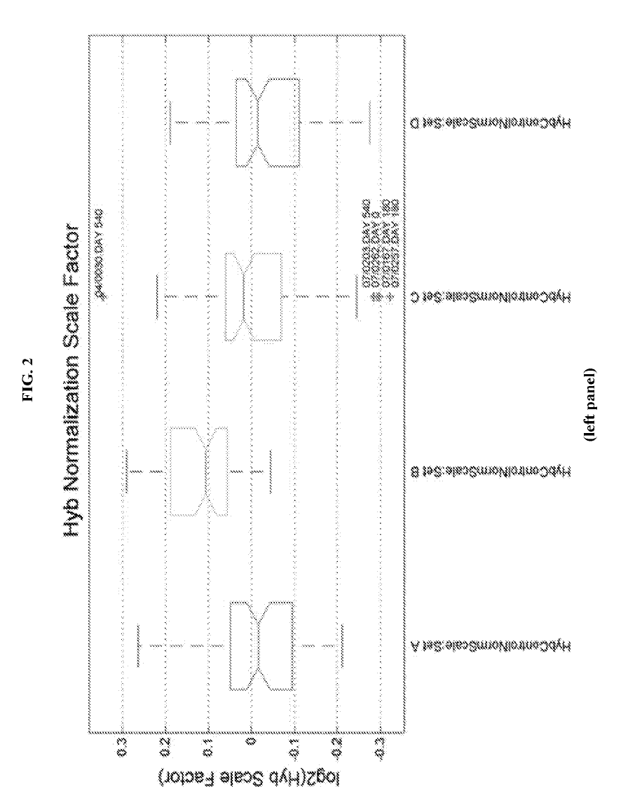

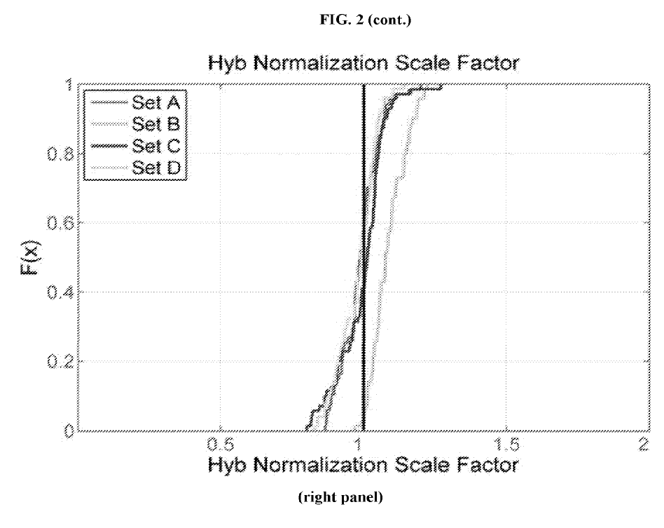

All Cases were diagnosed with active TB disease by 2 positive sputum smears and/or positive sputum culture. All TB cases have an exact date of treatment initiation with day zero being defined as the date of treatment initiation. FIG. 1 shows plots of sample time distributions for the discovery and verification sets.

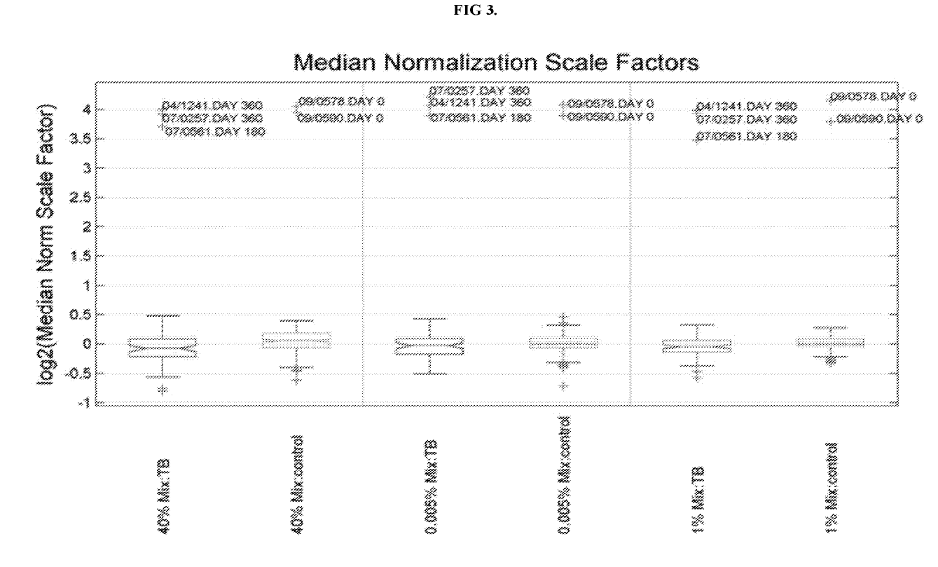

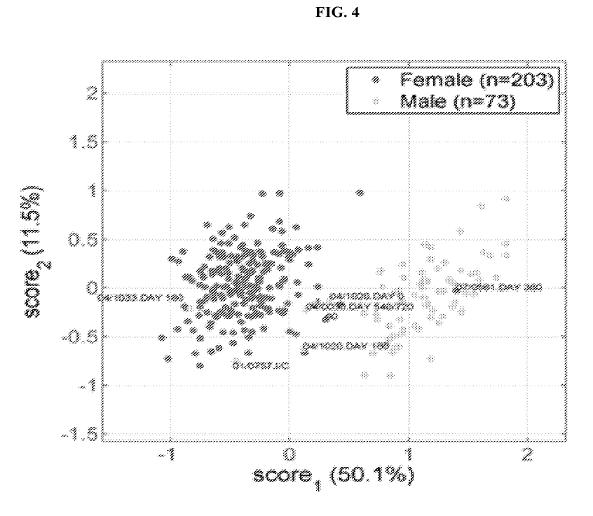

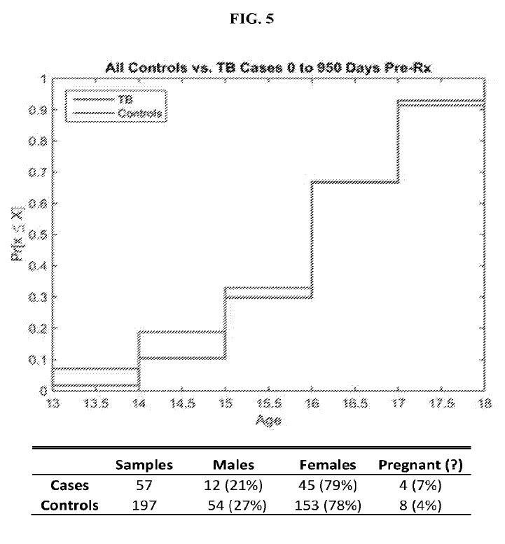

All samples were normalized and calibrated using standard hybridization and median normalization procedures. Hybridization normalization was performed using elution probes and is performed on a per sample basis. Hybridization scale factors are expected to be within the range 0.4-2.5, and all samples passed. As shown in FIG. 3, the median hybridization scale factor in each run is within 10% of unity except for plate B, which is slightly brighter compared to the other plates.

### Example 2

### Biomarker Discovery

### Two Group Diagnostic Comparison

All Data were log10 transformed to control for heteroscedasticity, thereby stabilizing the variance. The non-parametric Kolmogorov-Smirnov (KS) test was used to first compare TB Case and non-TB Control samples across all time points, then Case and matched Control groups were compared within the intervals 0-3, 6-12, and 12-18 months before the initiation of treatment. Hierarchical clustering was performed on all proteins by bin to determine which proteins have bin-dependent expression levels. This was further investigated using a generalized linear mixed model (GLMM) using the bin as a random effect. Linear regression was also performed on all TB cases to determine which proteins move linearly with time to diagnosis/treatment.

For each TB case within a time interval, controls were selected by matching bin number and study day. All samples were put into bins selected to control for various factors such as age, location, Mycobacterium tuberculosis exposure, etc. Stability selection using L1-regularized logistic regression was used to identify stable features in the presence of the available clinical covariates.

Prior to stratifying the samples by time to treatment, a cross-sectional comparison of all pre-treatment TB cases and all Control samples was performed using univariate KS. FIG. 5 shows demographic information for 57 TB Case samples (pre-treatment) and 197 Control samples used in the analysis. Comparing all 197 Control samples to all 57 TB case samples, 500 proteins were found to be significant at a 5% Benjamin-Hochberg False Discovery Rate (bhFDR). Of these, 348 proteins were higher in the TB cases and 152 were lower. Also significant at a 5% FDR were the HR9 proteins C9 (p=1.62e-6, rank 19), SERPINA4 (p=1.35e-4, rank 102), FCGR3B (p=1.4e4, rank 104) and SPOCK2 (p=7.22e-3, rank 467), as well as the TB-specific SOMAmers ESXA (p=1e-4, rank94), CH10 (p=7.36e-3, rank 471) and DNAK (p=2.3e-2, rank 739). Table 3 below shows the KS statistics for the top 25 ranked proteins.

TABLE 3
Top 25 ranked proteins differentiating all TB Cases from all non-TB
Controls. Proteins with positive KS distances are lower in TB cases.

| Rank | Target Name | UniProt ID | KS Dist. | p-value | pₑₘₚ | bhFDR |
|---|---|---|---|---|---|---|
| 1 | GALT1 | Q9NP70 | −0.492 | 6.60E−07 | 4.06E−10 | 0.0002 |
| 2 | AMBN | Q93038 | −0.454 | 6.60E−07 | 1.15E−08 | 0.0002 |
| 3 | C5 | Q10472 | −0.447 | 6.60E−07 | 1.97E−08 | 0.0002 |
| 4 | DR3 | Q8IXJ6 | −0.436 | 6.60E−07 | 4.85E−08 | 0.0002 |
| 5 | MMP-1 | Q86YW7 | −0.435 | 6.60E−07 | 5.21E−08 | 0.0002 |
| 6 | SAP | P01031 | −0.434 | 6.60E−07 | 5.84E−08 | 0.0002 |
| 7 | Angiopoietin-1 | Q6H9L7 | −0.432 | 6.60E−07 | 6.68E−08 | 0.0002 |
| 8 | Plasminogen | Q8TD33 | −0.419 | 6.60E−07 | 1.85E−07 | 0.0002 |
| 9 | NID2 | P03956 | −0.412 | 6.60E−07 | 3.15E−07 | 0.0002 |
| 10 | D-dimer | P28067 | −0.409 | 6.60E−07 | 4.19E−07 | 0.0002 |
| 11 | GPHB5 | O14896 | −0.409 | 6.60E−07 | 4.19E−07 | 0.0002 |
| 12 | SG1C1 | P02743 | −0.405 | 1.30E−06 | 5.69E−07 | 0.0003 |
| 13 | TGF-b3 | P00747 | −0.402 | 1.30E−06 | 6.76E−07 | 0.0003 |
| 14 | 2DMA | Q01459 | −0.402 | 1.30E−06 | 7.03E−07 | 0.0003 |
| 15 | Coagulation Factor X | P10600 | −0.401 | 1.30E−06 | 7.51E−07 | 0.0003 |
| 16 | C5b, 6 Complex | Q14112 | −0.399 | 2.00E−06 | 8.90E−07 | 0.0003 |
| 17 | IRF6 | P01031, P13671 | 0.397 | 2.00E−06 | 1.01E−06 | 0.0003 |
| 18 | Factor I | P02671 P02675 P02679 | −0.394 | 2.00E−06 | 1.25E−06 | 0.0003 |
| 19 | C9 | Q15389 | −0.39 | 2.60E−06 | 1.62E−06 | 0.0004 |
| 20 | SIRT2 | P02748 | 0.387 | 3.90E−06 | 1.98E−06 | 0.0005 |
| 21 | IP-10 | P05231 | −0.387 | 3.90E−06 | 2.11E−06 | 0.0005 |
| 22 | CLC6A | Q92575 | −0.387 | 3.90E−06 | 2.11E−06 | 0.0005 |
| 23 | PIM1 | Q6EIG7 | −0.386 | 3.90E−06 | 2.25E−06 | 0.0005 |
| 24 | DIAC | Q9Y6J6 | −0.385 | 4.60E−06 | 2.33E−06 | 0.0005 |
| 25 | IL-6 | P52943 | −0.385 | 4.60E−06 | 2.42E−06 | 0.0005 |

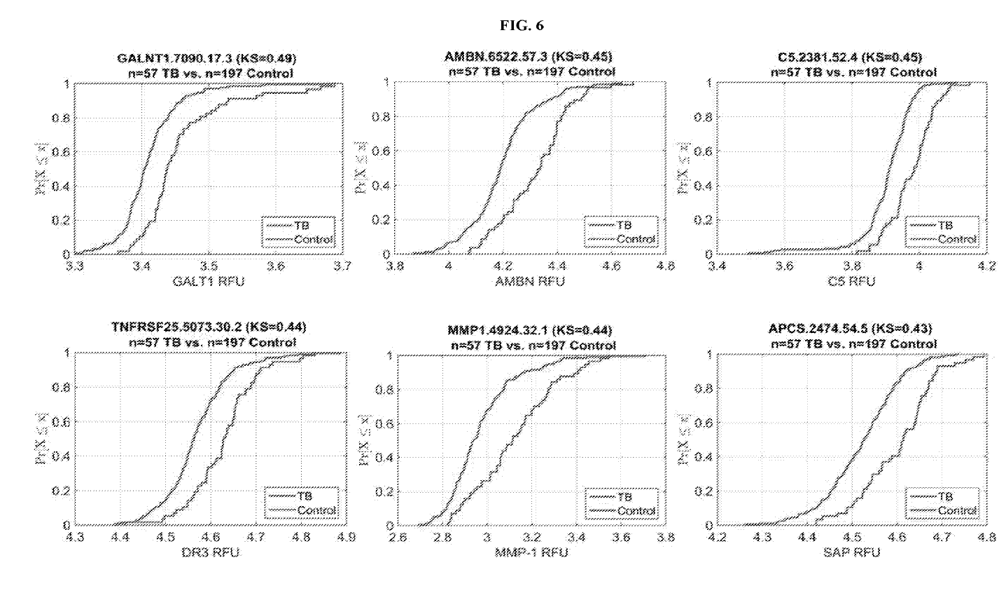

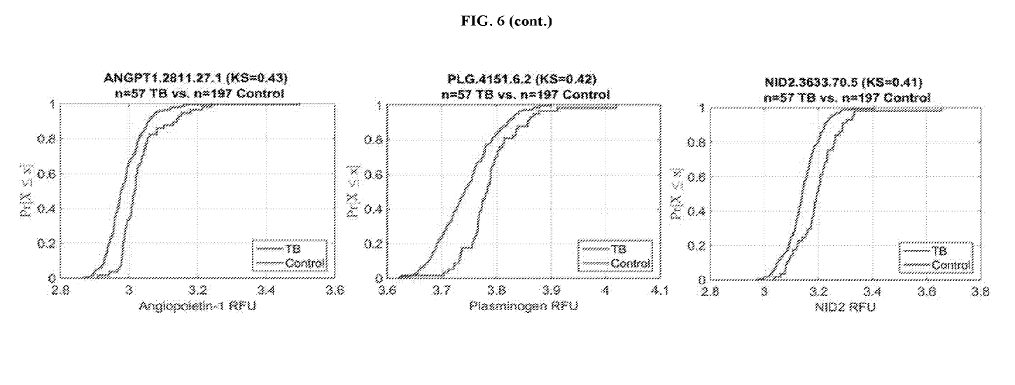

FIG. 6 shows the cumulative distribution functions (CDFs) for the top 9 proteins listed in the table above. P-values were calculated using a standard distribution (p-value) as well as an empirical null distribution created through class scrambling (Pemp).

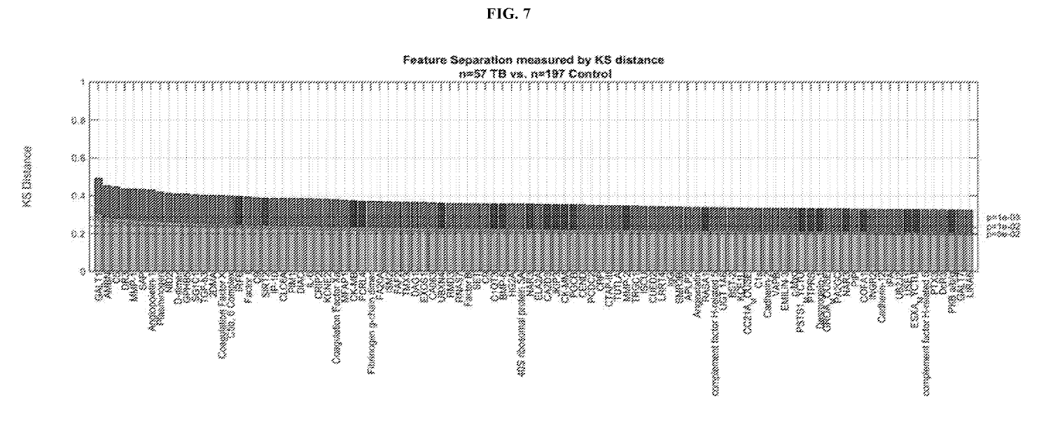

FIG. 7 shows the KS distances with class randomization statistics for the top 150 ranked proteins, which corresponded to a p-value cutoff of 1.35e-4. The total height of each bar represents the KS distance for the TB Case vs. Control comparison, with the top being green for proteins that are higher in TB Cases and red if they are lower. The height of the orange portion of each bar represents the median KS distance achieved through class randomization for that feature, and the error bars represent 95% confidence intervals.

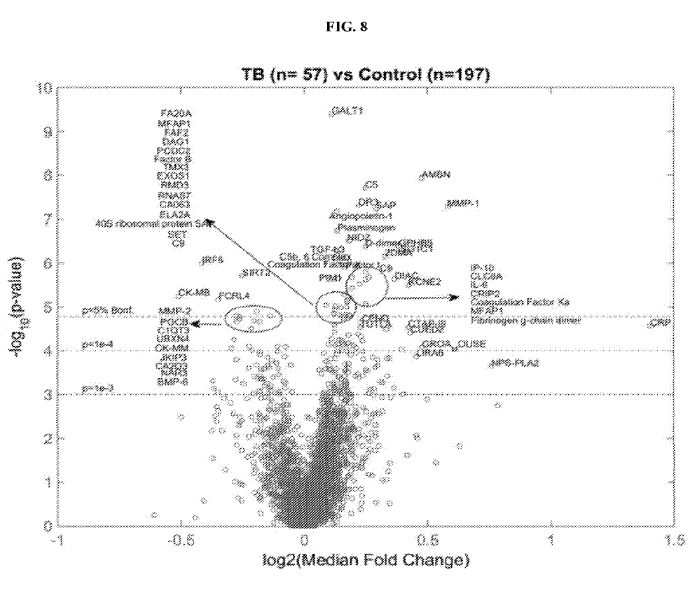

FIG. 8 shows a volcano plot of the negative log10-transformed p-values versus the log2 of the median TB RFU value over the median Control RFU value. A value of 1 on the horizontal axis corresponds to a 2-fold change in RFU.

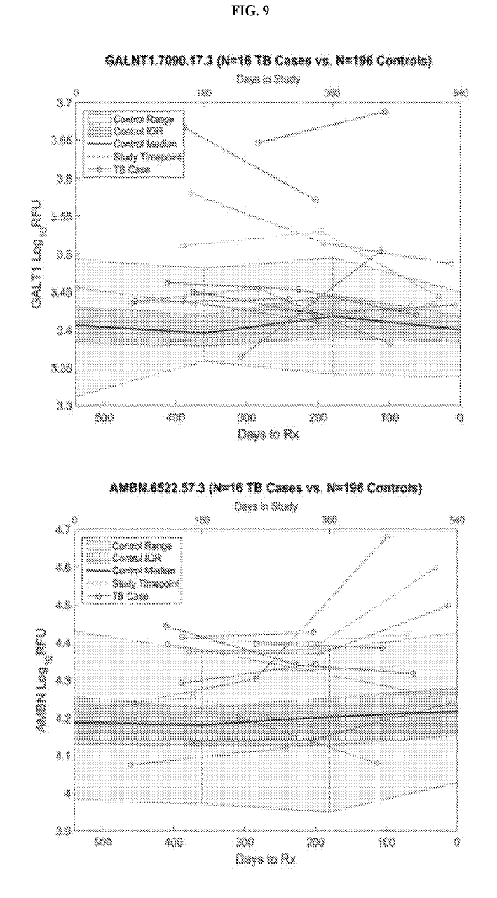

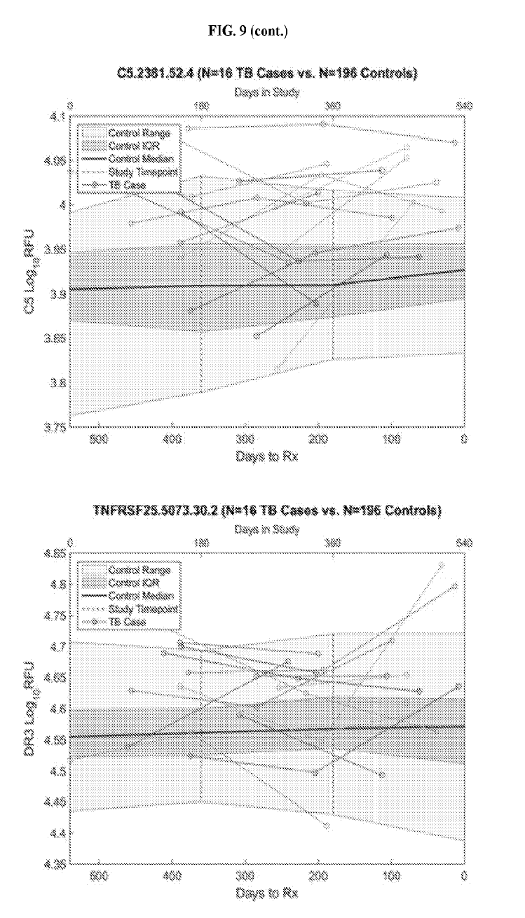

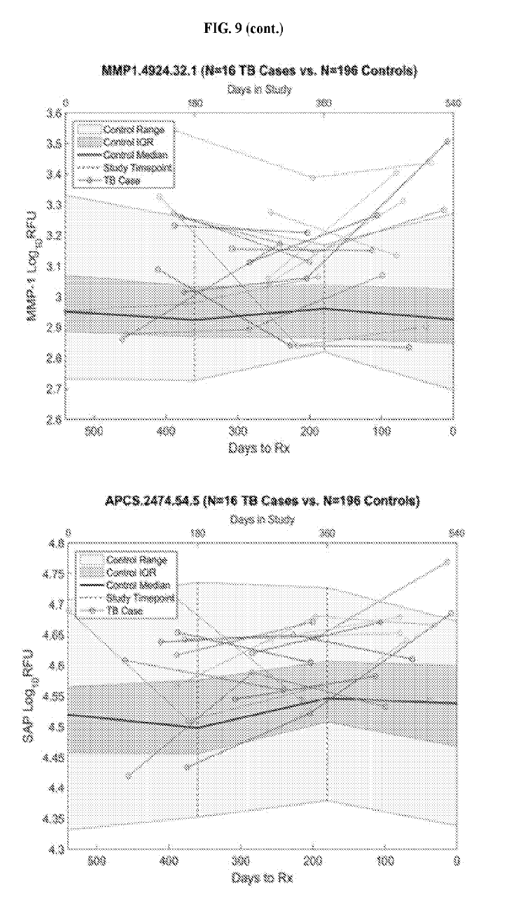

FIG. 9 shows the longitudinal RFU measurements for the 16 TB subjects with >1 time points overlaid on to a ‘control band’ created by interpolating the median, inter-quartile range and range of the control data. The control band is analogous to an interpolated boxplot of the RFU values of the Controls between days in study. The top axis (Days in Study) corresponds to the controls and the bottom axis (Days to Rx) corresponds to the TB cases. Time moves to the right in both groups.

### Hierarchical Clustering of Top Proteins within Each Bin

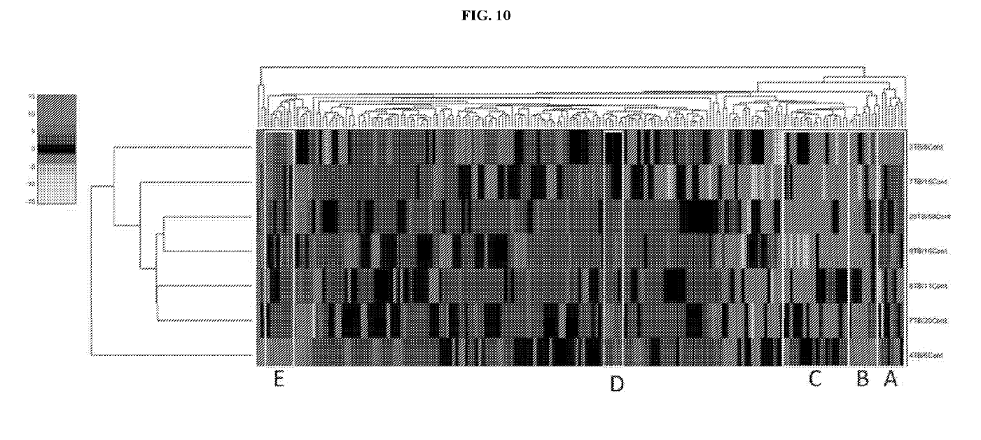

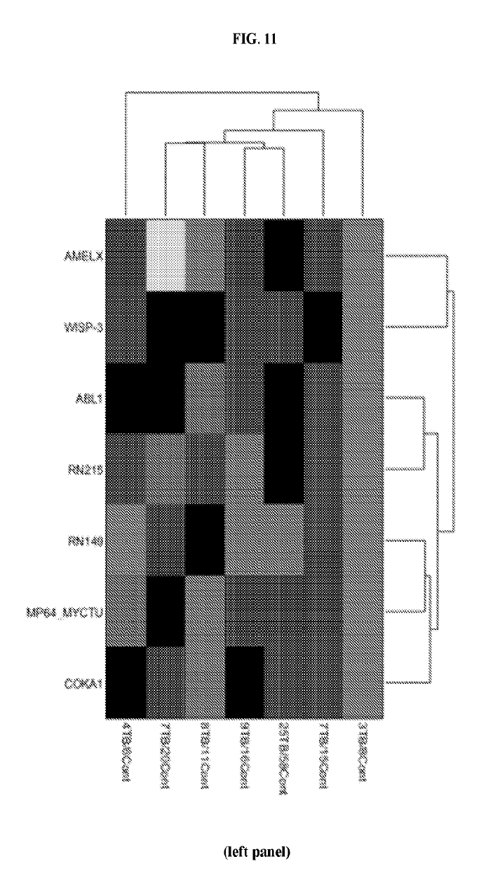

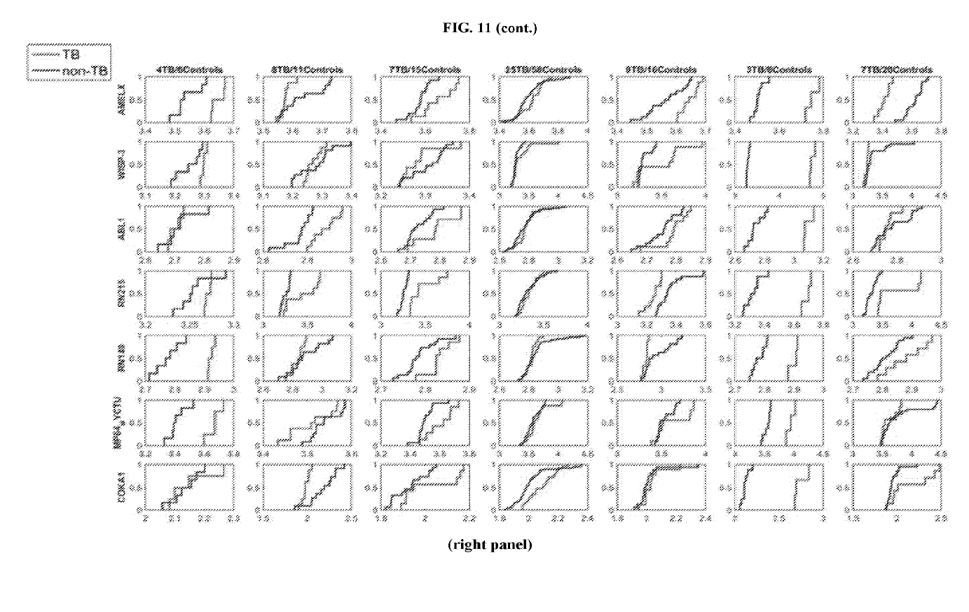

All samples were placed into 19 ‘bins’ which were matched according to age, high school, etc. Of these, 7 were observed to have >1 TB Case and >3 Controls. A t-test was used to find the magnitude of the intra-bin differences between TB Cases and Controls, and proteins were then ranked according to the median t-statistic across the 7 bins. Hierarchical clustering was performed on the top 200. A heat map of clustered t-statistics is shown in FIG. 10. The five regions in FIG. 10 marked A-E correspond to areas visually identified to have inconsistencies across the Bins.

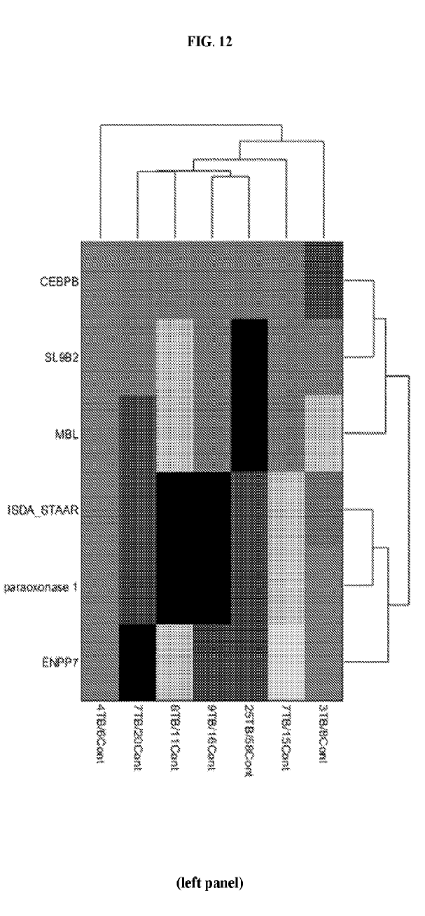

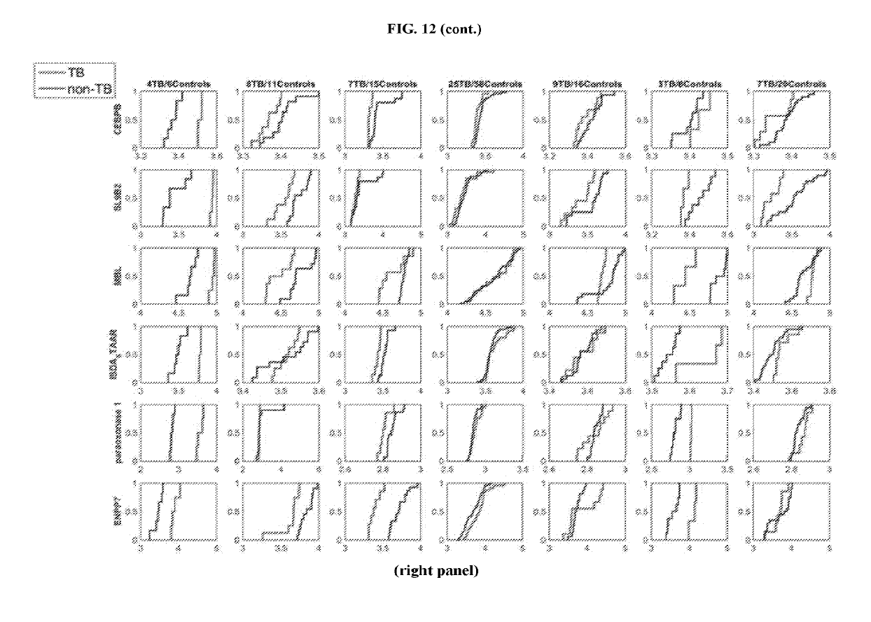

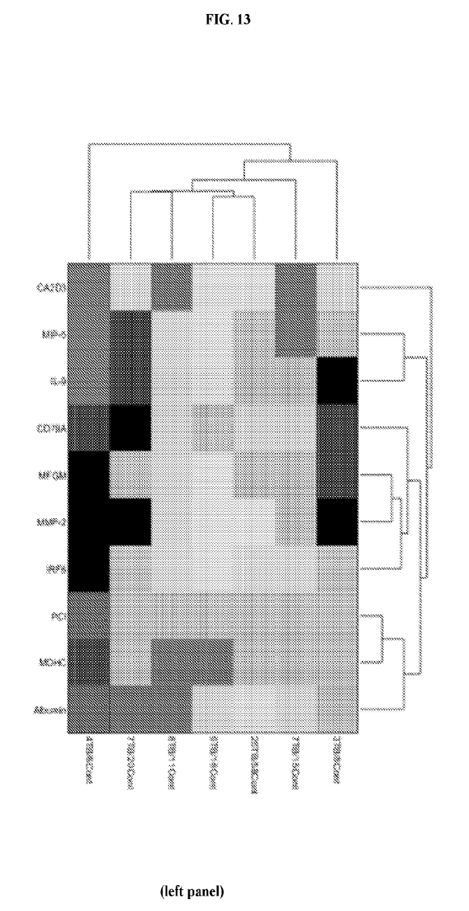

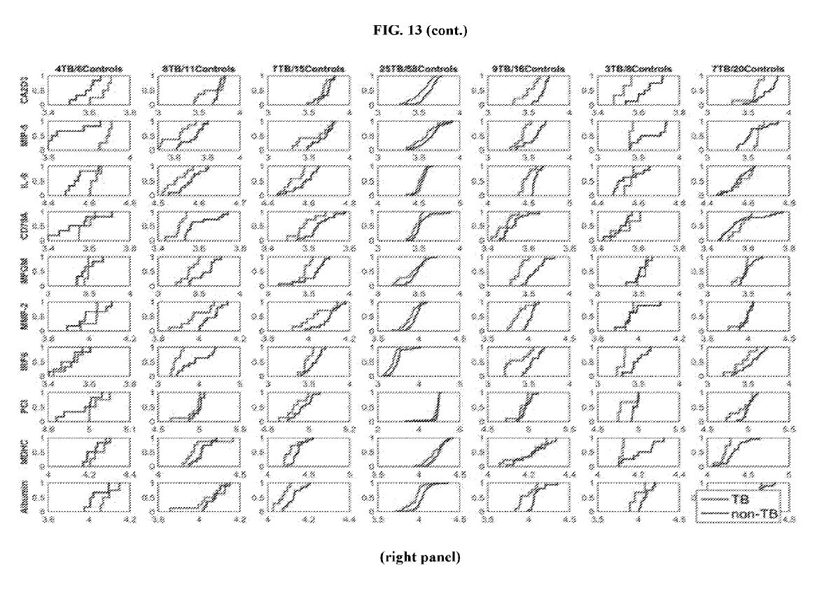

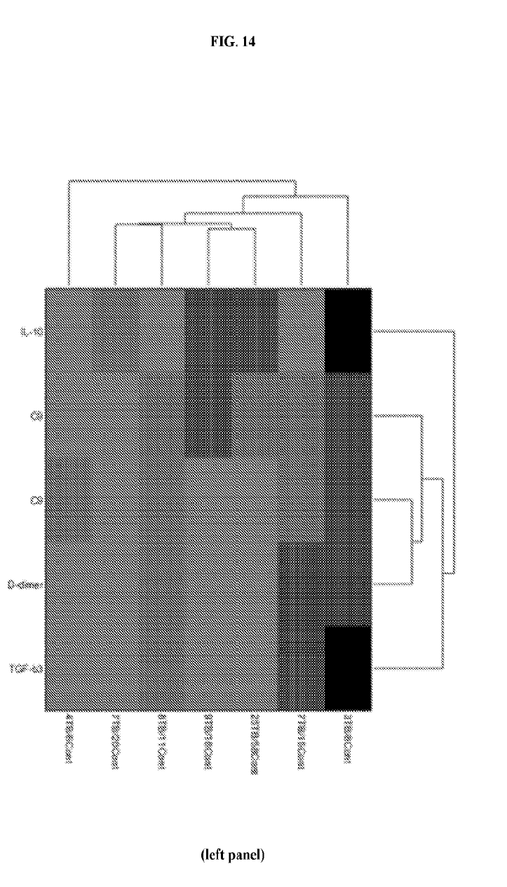

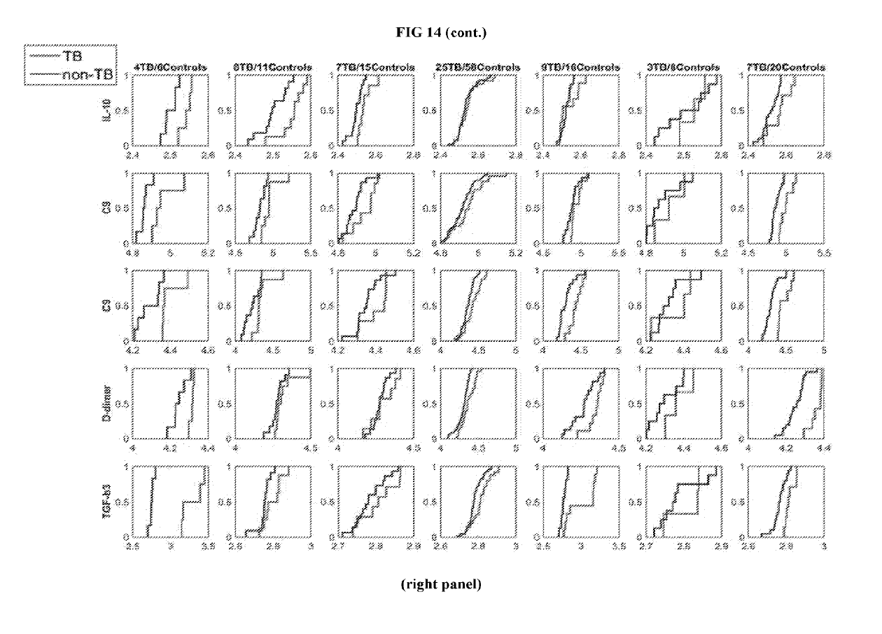

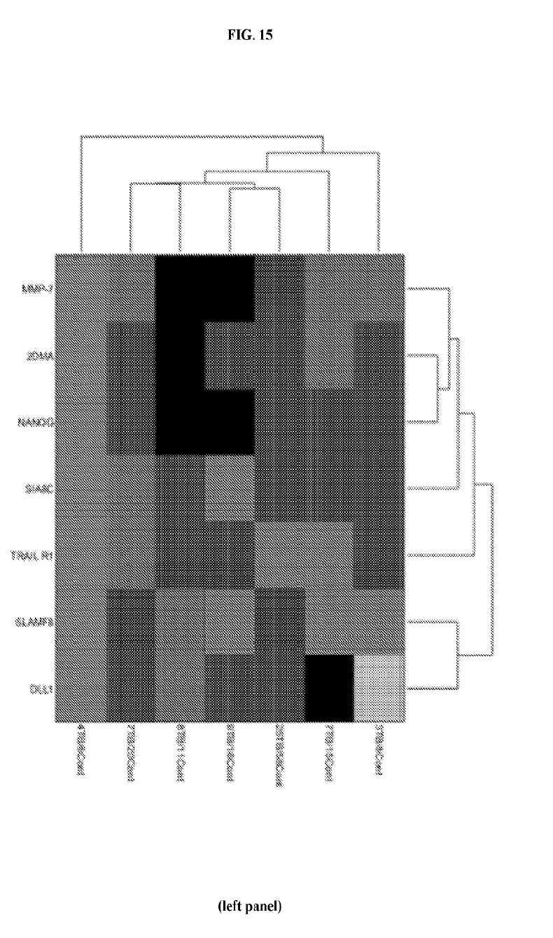

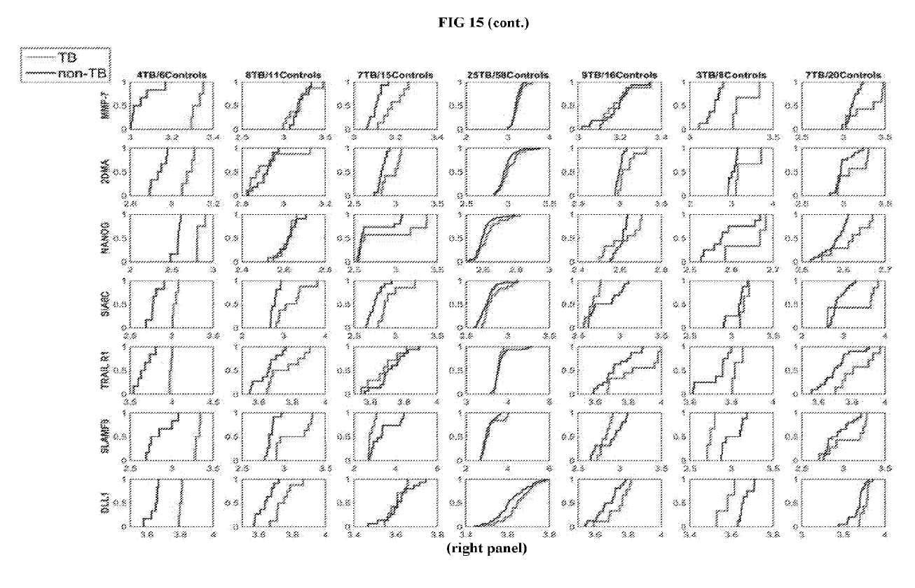

Hierarchical clustering arranges proteins according to similarities in expression. In FIGS. 11-15 (left), each row corresponds to a bin and each column a protein. Therefore, the coloring of each column represents the magnitude of the t-statistics for a particular protein across the 7 bins. Dendrograms (FIGS. 11-15, right) show the hierarchical grouping structure for regions marked A-E, with the height of each branch corresponding to the similarity between the underlying groups.

### Generalized Linear Mixed Effects Model

A generalized linear mixed effects model was used to determine the ability of each protein to classify subjects based on diagnosis while controlling for the bin number, as well as to determine which proteins have differences between the two groups which are dependent on the bin itself. Table 4 below shows statistics for the top 50 ranked proteins. A single p-value was generated for each protein (p_{fixed}) and was corrected for 3040 multiple comparisons (q_{fixed}). For each protein, 21 random effects p-value (p_{random}) were generate (one for each bin), and the minimum value is shown in the table. Overall, only proteins PDGFRA and SIA7A were found to have at least one bin that had a significant bin/random effect after correcting for multiple comparisons. At a 1% (p_{random}<0.01) uncorrected level, 327 proteins were found to have significant random effects. In the top 50 ranked proteins, none were found to have p_{random}<0.01. In the top 100 ranked proteins, only IL-12, STAT6 and Alpha-amylase 2B were found to have p_{random}<0.01.

TABLE 4
Generalized linear mixed model statistics
comparing all 57 TB cases to 197 Controls.

| | | | | min | LogLikeli- | |
|---|---|---|---|---|---|---|
| Rank | Target | p_{fixed} | q_{fixed} | (p_{random}) | hood | r\hat{ }2 |
| 1 | IRF6 | 1.64E−07 | 1.25E−04 | 1.000 | −632.9 | 0.28 |
| 2 | C9 | 3.20E−07 | 1.95E−04 | 1.000 | −621.8 | 0.13 |
| 3 | MMP-2 | 4.18E−07 | 2.12E−04 | 0.321 | −610.0 | 0.13 |
| 4 | Factor I | 6.15E−07 | 2.67E−04 | 1.000 | −617.4 | 0.13 |
| 5 | D-dimer | 1.08E−06 | 4.11E−04 | 0.255 | −639.1 | 0.22 |
| 6 | C5 | 2.69E−06 | 9.08E−04 | 0.067 | −648.3 | 0.29 |
| 7 | CK-MB | 3.05E−06 | 9.26E−04 | 0.327 | −614.8 | 0.19 |
| 8 | Albumin | 4.53E−06 | 1.25E−03 | 1.000 | −613.1 | 0.12 |
| 9 | Fibrinogen | 5.01E−06 | 1.27E−03 | 0.422 | −618.8 | 0.20 |
| | g-chain | | | | | |
| | dimer | | | | | |
| 10 | CRP | 7.67E−06 | 1.79E−03 | 0.349 | −606.2 | 0.12 |
| 11 | SAP | 8.85E−06 | 1.92E−03 | 0.017 | −618.9 | 0.25 |
| 12 | C9 | 1.10E−05 | 2.23E−03 | 0.498 | −603.7 | 0.18 |
| 13 | SET | 1.35E−05 | 2.57E−03 | 1.000 | −602.6 | 0.09 |
| 14 | CK-MM | 1.47E−05 | 2.63E−03 | 1.000 | −624.3 | 0.17 |
| 15 | MDC | 2.68E−05 | 4.53E−03 | 0.444 | −601.5 | 0.12 |
| 16 | MDHC | 3.04E−05 | 4.86E−03 | 1.000 | −612.1 | 0.10 |
| 17 | FGL1 | 3.54E−05 | 5.39E−03 | 1.000 | −597.2 | 0.07 |
| 18 | TGF-b3 | 4.11E−05 | 5.95E−03 | 1.000 | −625.2 | 0.81 |
| 19 | Dynactin | 5.48E−05 | 7.57E−03 | 1.000 | −603.1 | 0.07 |
| | subunit 2 | | | | | |
| 20 | Plasmin- | 5.78E−05 | 7.64E−03 | 0.350 | −601.4 | 0.11 |
| | ogen | | | | | |
| 21 | LRRT3 | 6.83E−05 | 8.65E−03 | 0.177 | −604.3 | 0.26 |
| 22 | PIP | 7.20E−05 | 8.76E−03 | 1.000 | −600.6 | 0.09 |
| 23 | MMP-1 | 7.81E−05 | 9.13E−03 | 0.110 | −605.4 | 0.18 |
| 24 | Factor B | 8.41E−05 | 9.15E−03 | 1.000 | −600.7 | 0.07 |
| 25 | FCN1 | 8.43E−05 | 9.15E−03 | 1.000 | −594.5 | 0.06 |

The top 100 proteins ranked by KS distance and stability selection were also investigated for proteins with significant bin effects. 15 proteins in the KS ranked list and 24 in the stability selection ranked list were found to have p_{random}<0.01, and none were significant after correcting for multiple comparisons.

### Linear Regression

In order to find proteins which respond to the progression of TB pathogenesis, a linear regression was run on all TB case samples taken <300 before the beginning of treatment. This time window was chosen because protein signals were observed to generally stabilize >300 days pre-treatment, which would confound regression statistics. Table 5 below shows regression statistics for RFU level as a function of days to initiation of treatment.

TABLE 5
Linear regression statistics for all TB Case samples <
300 days before treatment initiation.

| Rank | Target | Beta | R\hat{ }2 | p-value | q-value |
|---|---|---|---|---|---|
| 1 | IMPA3 | 0.001 | 0.428 | 6.62E−05 | 0.142 |
| 2 | B4GT6 | 0.001 | 0.413 | 9.72E−05 | 0.142 |
| 3 | NLGN2 | 0.001 | 0.399 | 1.40E−04 | 0.142 |
| 4 | C9 | −0.001 | 0.351 | 4.46E−04 | 0.306 |
| 5 | C9 | −0.001 | 0.345 | 5.09E−04 | 0.306 |
| 6 | BOC | 0.001 | 0.338 | 6.04E−04 | 0.306 |
| 7 | PTPRD | 0.001 | 0.312 | 1.08E−03 | 0.410 |
| 8 | CRP | −0.002 | 0.308 | 1.19E−03 | 0.410 |
| 9 | CA2D3 | 0.001 | 0.304 | 1.29E−03 | 0.410 |
| 10 | IL-11 RA | 0.001 | 0.303 | 1.35E−03 | 0.410 |
| 11 | OMD | 0.001 | 0.293 | 1.68E−03 | 0.462 |
| 12 | CA226 | −0.001 | 0.289 | 1.82E−03 | 0.462 |
| 13 | Cathelicidin peptide | −0.001 | 0.276 | 2.42E−03 | 0.543 |
| 14 | F150B | 0 | 0.273 | 2.54E−03 | 0.543 |
| 15 | Lymphotoxin a1/b2 | 0 | 0.267 | 2.94E−03 | 0.543 |
| 16 | BMP-6 | 0.001 | 0.264 | 3.10E−03 | 0.543 |
| 17 | Periostin | 0.001 | 0.261 | 3.32E−03 | 0.543 |
| 18 | NDST1 | 0 | 0.256 | 3.66E−03 | 0.543 |
| 19 | C3b | −0.002 | 0.249 | 4.23E−03 | 0.543 |
| 20 | IL-19 | 0.001 | 0.247 | 4.42E−03 | 0.543 |
| 21 | EMIL3 | 0.001 | 0.245 | 4.68E−03 | 0.543 |
| 22 | PolyUbiquitin K48 | 0 | 0.24 | 5.18E−03 | 0.543 |
| 23 | PKB a/b/g | 0 | 0.239 | 5.26E−03 | 0.543 |
| 24 | TSP4 | 0.001 | 0.238 | 5.36E−03 | 0.543 |
| 25 | Factor B | 0 | 0.237 | 5.47E−03 | 0.543 |

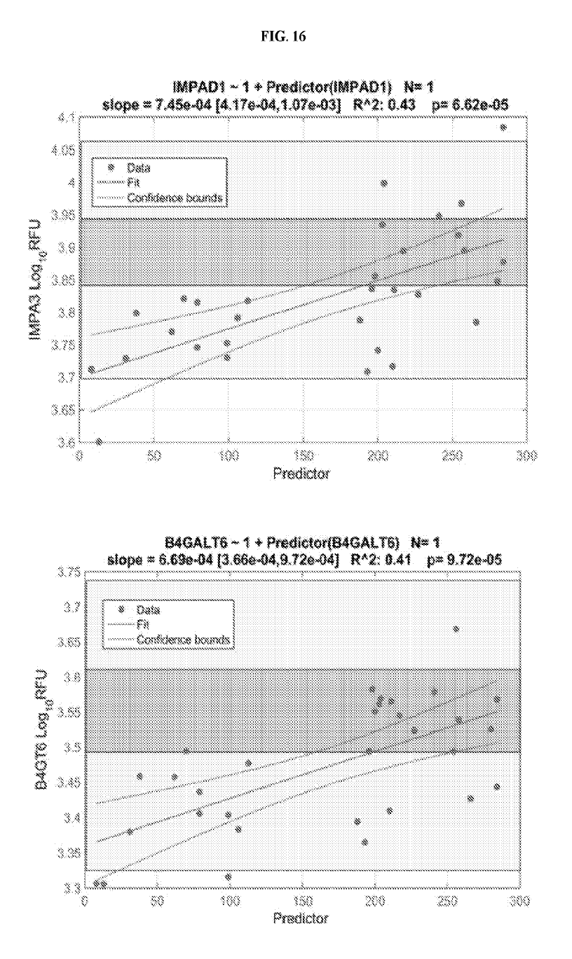

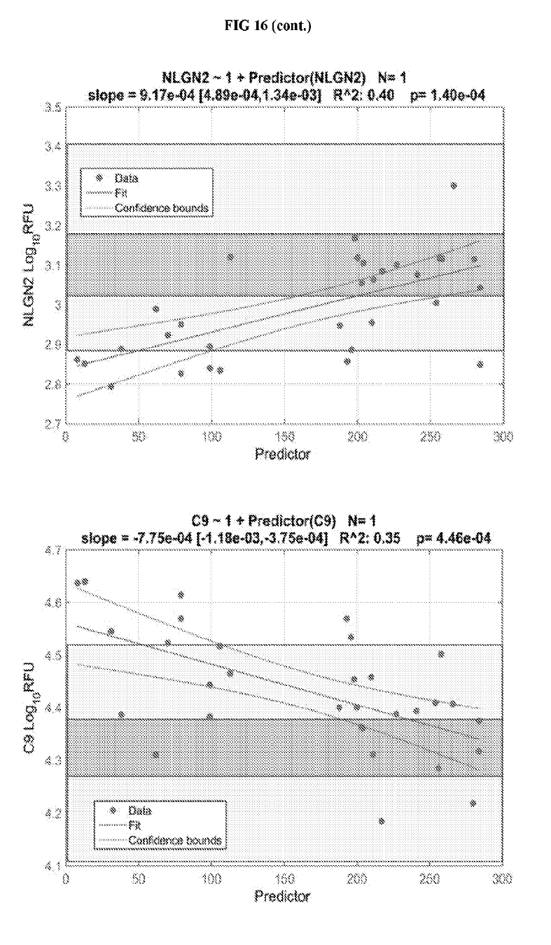

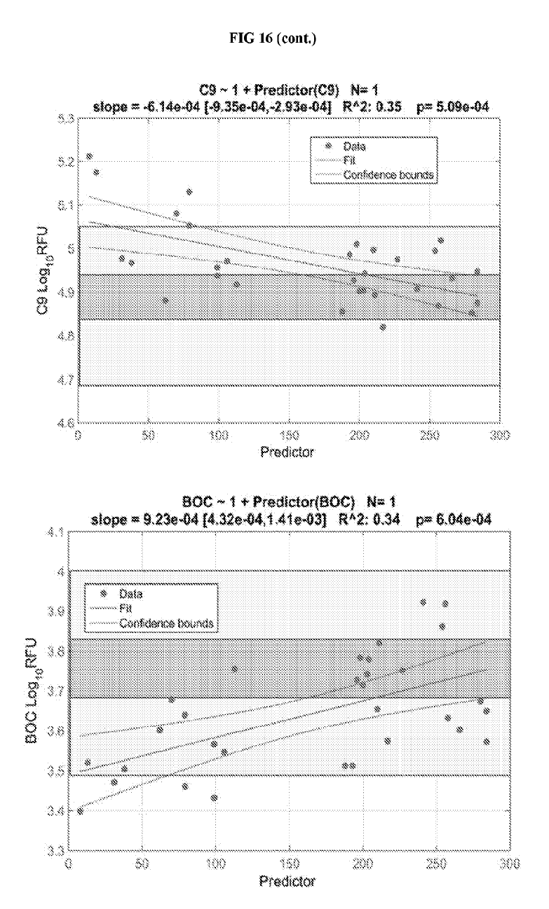

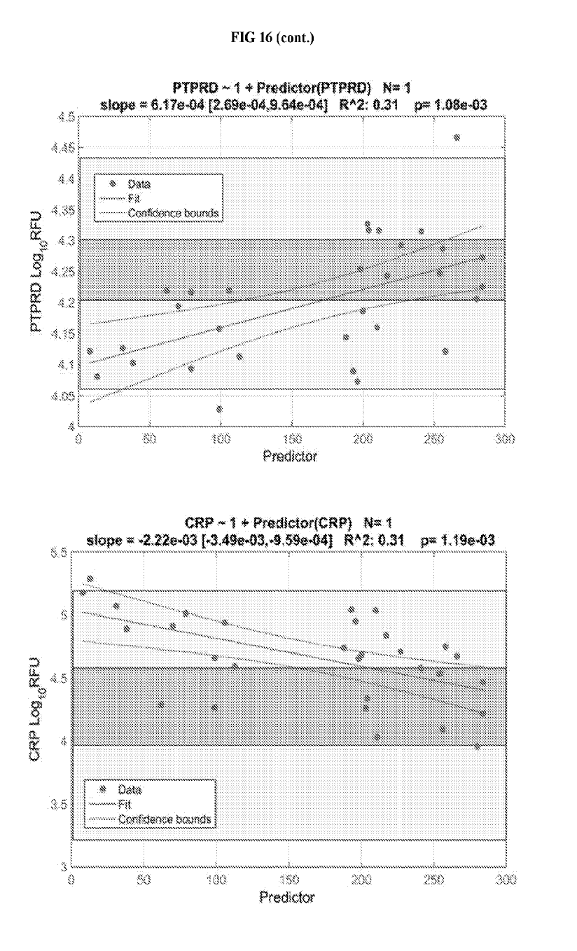

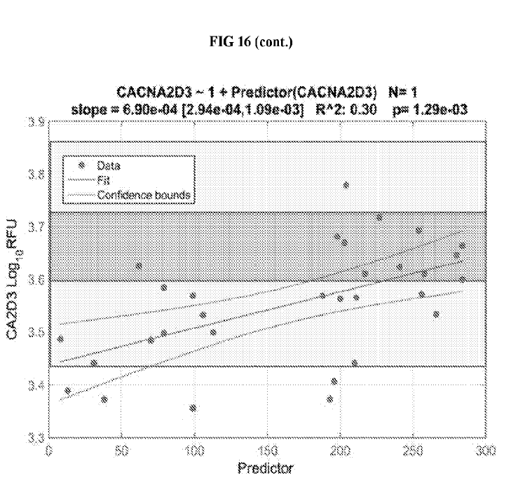

FIG. 16 shows scatter plots for the top 9 ranked proteins from a linear regression. Linear fits for all TB cases are shown as a function of time to treatment, with time moving to the left. To provide a notion for how well these proteins distinguish the TB from non-TB cohorts, this information is overlaid onto data representing a boxplot for all control RFU data. The dark band corresponds to the interquartile range (IQR), while the lighter shaded region corresponds to the whiskers, or the nearest data point that's within the upper/lower quartile+1.5*IQR.

### Time Point Stratification

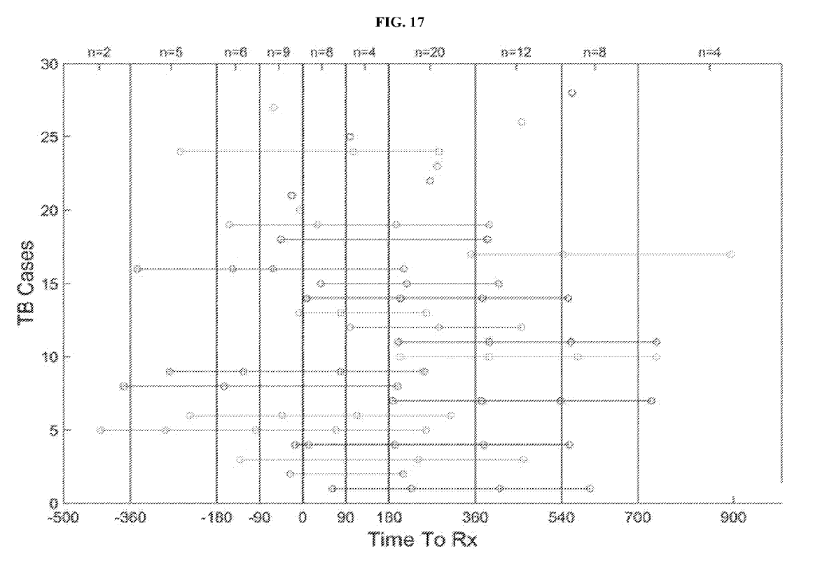

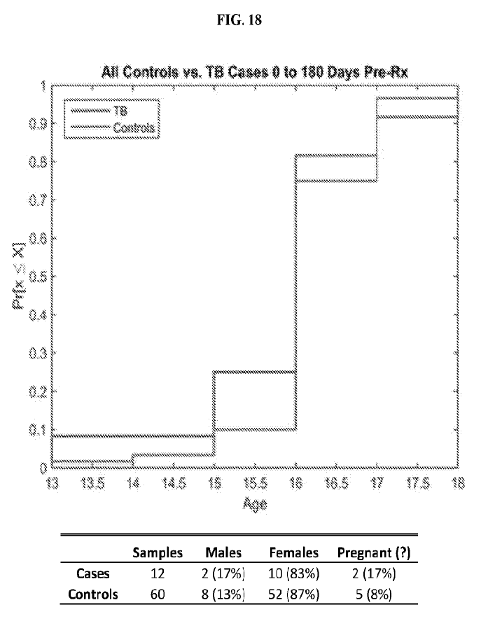

In order to break the serial sampling structure in the data which leads to a decreased estimate of the true variance, the data was stratified into time bins and a univariate analysis was repeated. Time bins were created based on the distribution of non-repeated subjects within each time interval. FIG. 17 shows the Time to treatment (Rx) for each TB case. Based on these criteria, intervals of 0 to 180 days (n=12), 180 to 360 days (n=20), 360 to 540 days (n=12) and 540-700 were chosen. For all TB cases within a given time range, a control population was constructed based on the bin and study day. For a given case, the Control samples from its bin with a matching study day were selected. There were typically 1-3 matched controls for each case. However, since the controls within a bin are typically serial samples from control subjects, if TB cases are from multiple study days within a given bin then serial sampling of the controls is not broken. This will underestimate the true variance of the control population. FIG. 17 shows sample times for all TB subjects as a function of time to the beginning of treatment. FIG. 18 shows demographics for TB Cases 0 to 180 days pre-treatment and matched controls.

Comparing 12 TB cases 0 to 180 days before diagnosis and treatment with all 60 matched non-TB Controls, a KS test identified 30 proteins to be differentially expressed between TB and non-TB subjects at 5% Benjamini-Hochberg False Discovery Rate (bhFDR). This includes the protein C9 (p=3.233-3, rank 5), which has previously been shown to be diagnostic for TB infection and/or disease. Nine of the 30 proteins were higher in the TB. At a 10% bhFDR 82 proteins were significant with 29 being higher in the TB group, and at a 20% FDR 303 proteins were significant with 128 of those being higher in the TB group. Table 6 below shows KS statistics for the top 25 ranked proteins.

TABLE 6
Top 25 ranked proteins differentiating TB Cases 0-180 days pre-treatment from matched
non-TB Controls. Proteins with positive KS distances are lower in TB cases.

| | Target | UniProt | KS | | | |
|---|---|---|---|---|---|---|
| Rank | Name | ID | Dist. | p-value | pₑₘₚ | bhFDR |
| 1 | MMP-2 | P08253 | 0.8 | 6.60E−07 | 1.53E−06 | 0.002 |
| 2 | CLFB_STAAE | O86476 | 0.717 | 1.80E−05 | 2.47E−05 | 0.026 |
| 3 | IL-6 | P05231 | −0.7 | 3.00E−05 | 4.15E−05 | 0.028 |
| 4 | C1QT3 | Q9BXJ4 | 0.667 | 6.90E−05 | 1.13E−04 | 0.037 |
| 5 | C9 | P02748 | −0.65 | 1.50E−04 | 1.83E−04 | 0.037 |
| 6 | EDA | Q92838 | 0.65 | 1.50E−04 | 1.83E−04 | 0.037 |
| 7 | D-dimer | P02671 P02675 | −0.65 | 1.50E−04 | 1.83E−04 | 0.037 |
| | | P02679 | | | | |
| 8 | CA2D3 | Q8IZS8 | 0.65 | 1.50E−04 | 1.83E−04 | 0.037 |
| 9 | RAD51 | Q06609 | 0.65 | 1.70E−04 | 1.83E−04 | 0.037 |
| 10 | RSPO4 | Q2I0M5 | −0.65 | 1.70E−04 | 1.83E−04 | 0.037 |
| 11 | MRP6 | O95255 | 0.65 | 1.70E−04 | 1.83E−04 | 0.037 |
| 12 | AMBN | Q9NP70 | −0.633 | 1.80E−04 | 2.94E−04 | 0.037 |
| 13 | B4GT6 | Q9UBX8 | 0.633 | 2.80E−04 | 2.94E−04 | 0.037 |
| 14 | PGCB | Q96GW7 | 0.633 | 2.80E−04 | 2.94E−04 | 0.037 |
| 15 | IgG | P01857 | −0.633 | 2.80E−04 | 2.94E−04 | 0.037 |
| 16 | Fibrinogen g-chain | P02679 | −0.633 | 2.80E−04 | 2.94E−04 | 0.037 |
| | dimer | | | | | |
| 17 | Nr-CAM | Q92823 | 0.633 | 2.80E−04 | 2.94E−04 | 0.037 |
| 18 | CBPE | P16870 | 0.633 | 2.80E−04 | 2.94E−04 | 0.037 |
| 19 | MED-1 | Q15648 | 0.633 | 2.80E−04 | 2.94E−04 | 0.037 |
| 20 | NPS | P0C0P6 | 0.633 | 2.80E−04 | 2.94E−04 | 0.037 |
| 21 | NLGN2 | Q8NFZ4 | 0.633 | 2.80E−04 | 2.94E−04 | 0.037 |
| 22 | WFKN2 | Q8TEU8 | 0.617 | 4.50E−04 | 4.64E−04 | 0.042 |
| 23 | SIRT2 | Q8IXJ6 | 0.617 | 4.50E−04 | 4.64E−04 | 0.042 |
| 24 | PKB beta | P31751 | 0.617 | 4.50E−04 | 4.64E−04 | 0.042 |
| 25 | Ephrin-A3 | P52797 | 0.617 | 4.50E−04 | 4.64E−04 | 0.042 |

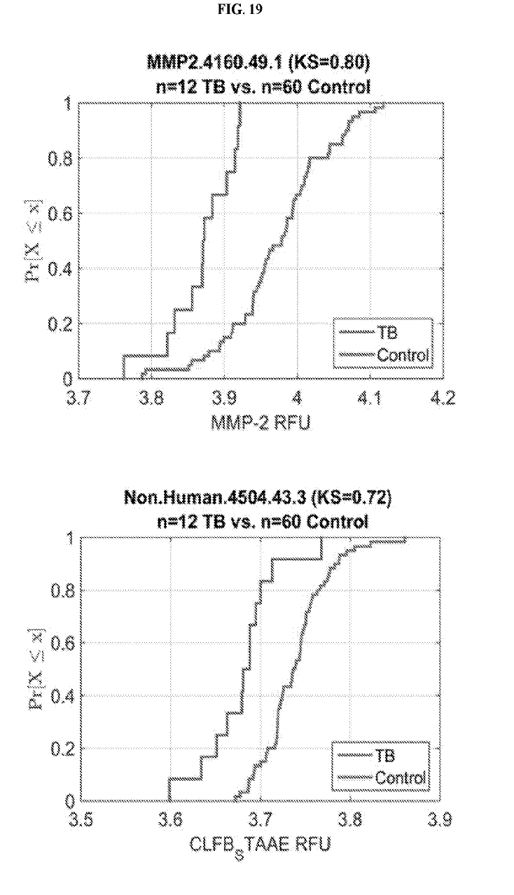

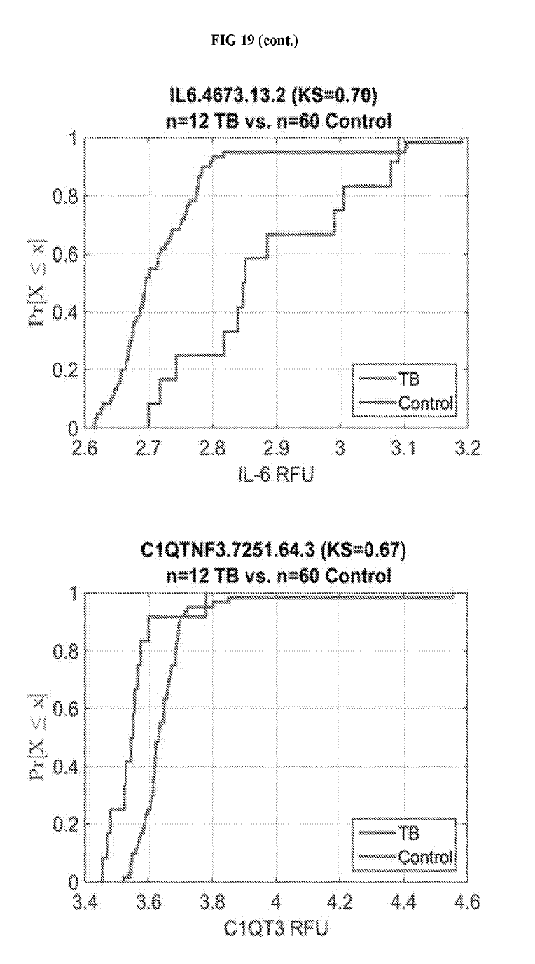

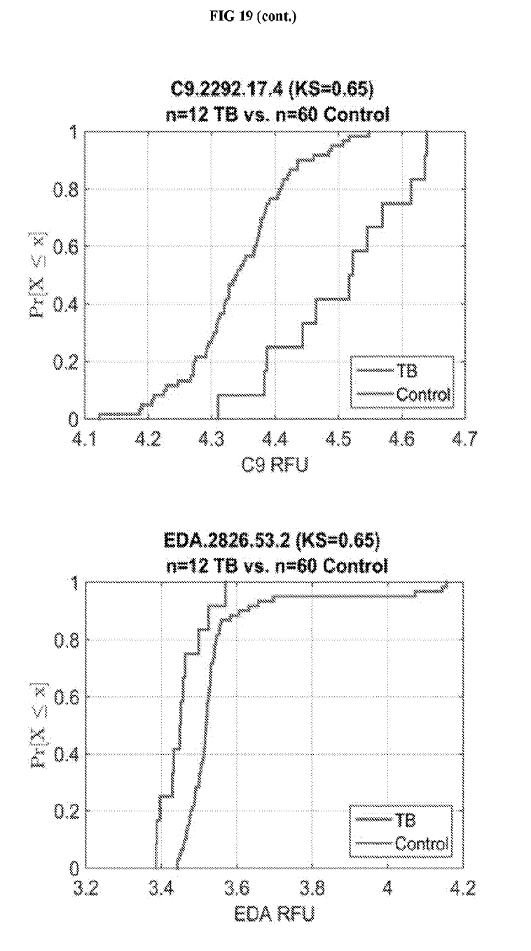

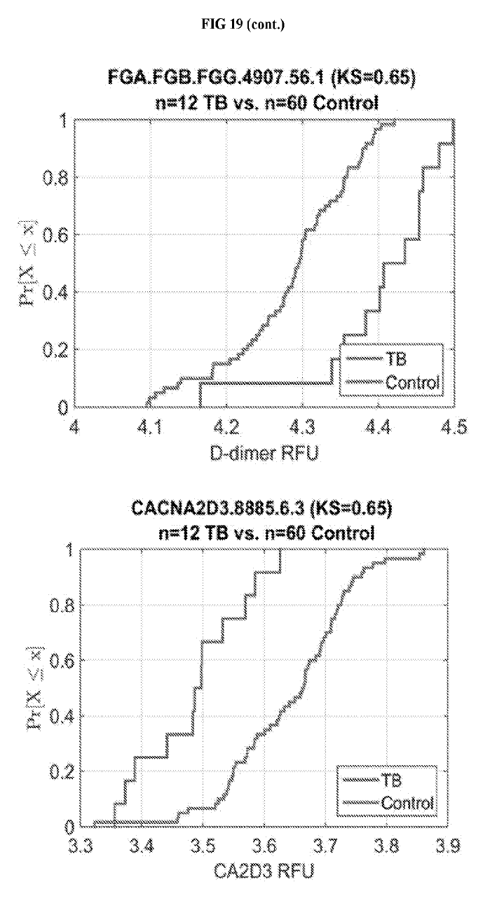

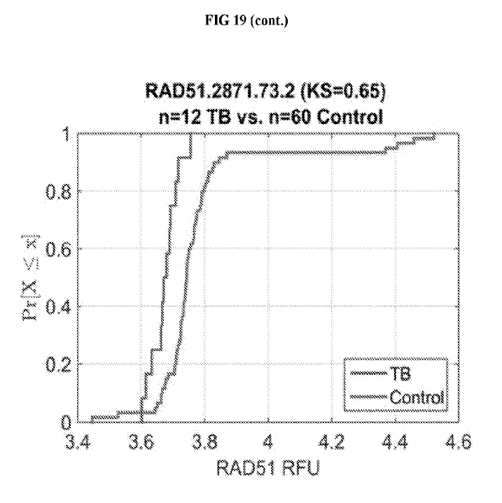

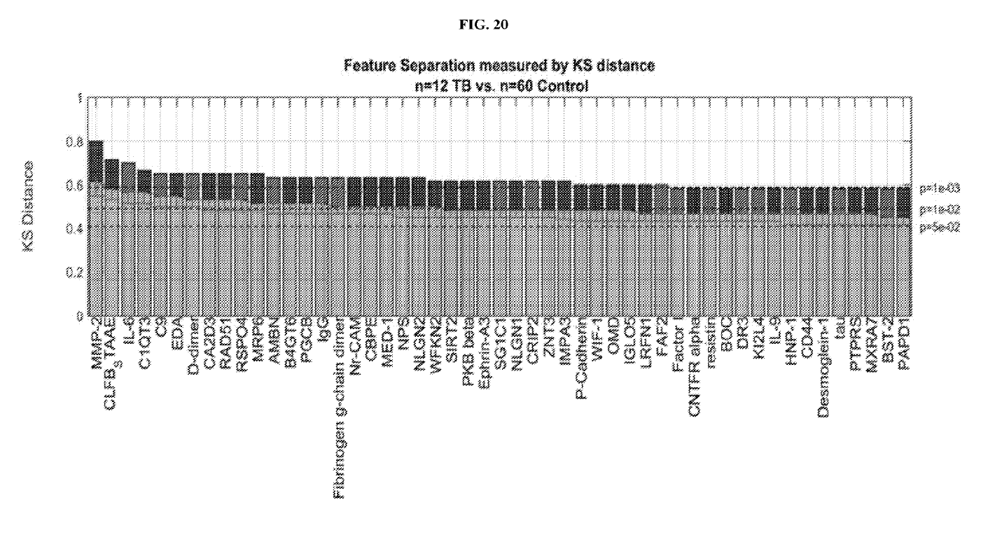

The second ranked protein CLFB_STAAE is a Staphylococcus aureus protein. FIG. 19 shows CDFs for the top 9 ranked proteins, including CLFB_STAAE, and FIG. 20 shows KS distances with class randomization statistics for the top 100 ranked proteins, which corresponded to a p-value cutoff of 1.12e-3.

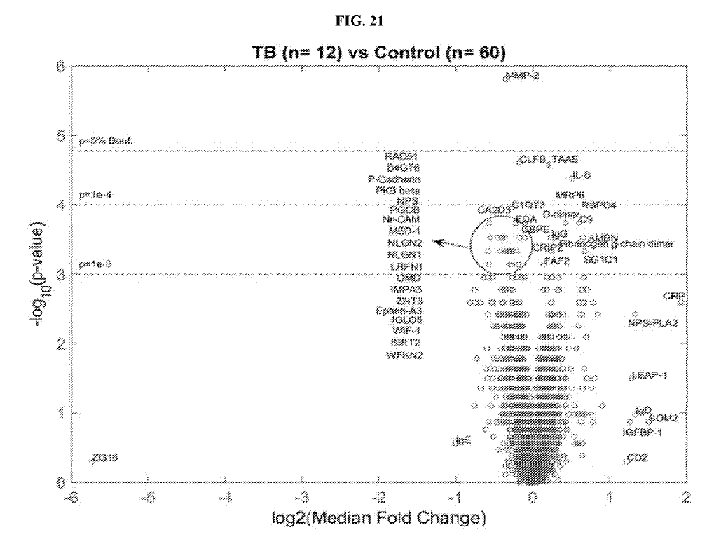

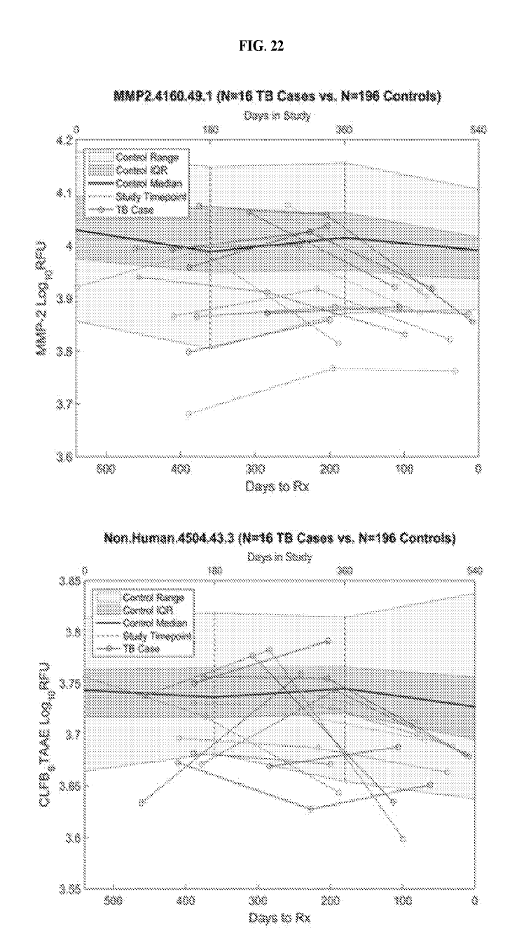

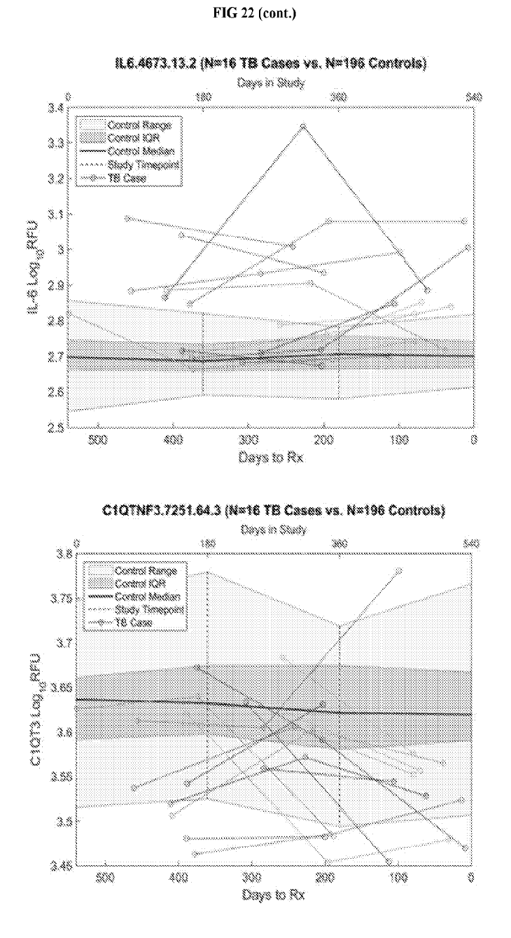

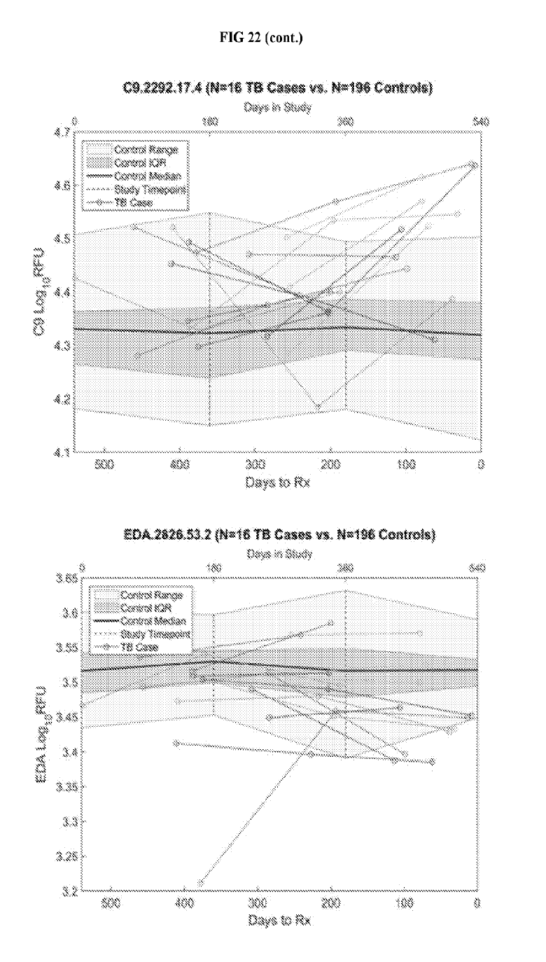

FIG. 21 shows a volcano plot of the negative log10-transformed p-values versus the log2 of the median TB RFU value over the median Control RFU value. A value of 1 on the horizontal axis corresponds to a 2-fold change in RFU. FIG. 22 shows control band plots for the top 6 ranked proteins.

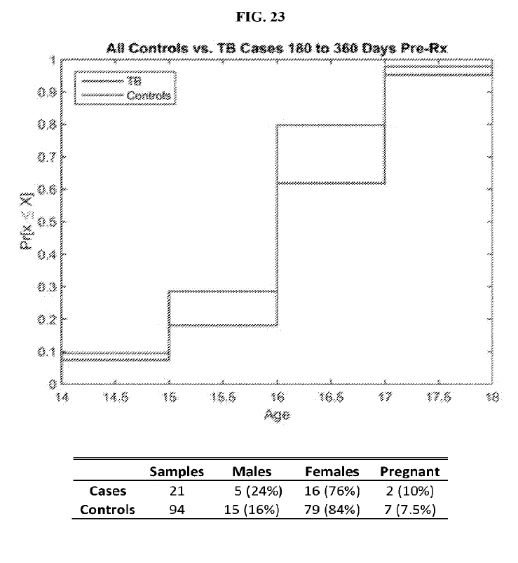

FIG. 23 shows demographics for the controls at all time-points as well as the TB cases 180 to 360 days pre-treatment.

Comparing 21 TB cases to 94 matched non-TB Controls, a KS test identified 0 proteins to be differentially expressed between TB and non-TB subjects at 5% FDR. 16 proteins were found to be significant at a 20% FDR with 14 having higher expression in the TB group. None of the known TB-specific were observed to have an FDR<40%.

TABLE 7
Top 25 ranked proteins differentiating TB Cases 180-360
days pre- treatment from matched non-TB Controls. Proteins
with positive KS distances are lower in TB cases.

| | Target | UniProt | KS | | | |
|---|---|---|---|---|---|---|
| Rank | Name | ID | Dist. | p-value | pₑₘₚ | bhFDR |
| 1 | ISM2 | Q6H9L7 | −0.539 | 3.20E−05 | 4.64E−05 | 0.084 |
| 2 | AMBN | Q9NP70 | −0.523 | 6.90E−05 | 8.56E−05 | 0.084 |
| 3 | SG1C1 | Q8TD33 | −0.517 | 8.60E−05 | 1.06E−04 | 0.084 |
| 4 | DIAC | Q01459 | −0.486 | 2.80E−04 | 3.38E−04 | 0.185 |
| 5 | RNAS7 | Q9H1E1 | −0.475 | 4.20E−04 | 4.92E−04 | 0.185 |
| 6 | MMP-1 | P03956 | −0.475 | 4.40E−04 | 5.01E−04 | 0.185 |
| 7 | DR3 | Q93038 | −0.47 | 4.90E−04 | 5.87E−04 | 0.185 |
| 8 | IRF6 | O14896 | 0.469 | 5.40E−04 | 6.08E−04 | 0.185 |
| 9 | RMD3 | Q96TC7 | −0.465 | 6.00E−04 | 7.11E−04 | 0.185 |
| 10 | DAG1 | Q14118 | −0.458 | 8.20E−04 | 8.74E−04 | 0.185 |
| 11 | GPHB5 | Q86YW7 | −0.454 | 8.60E−04 | 1.00E−03 | 0.185 |
| 12 | TGF-b3 | P10600 | −0.453 | 9.30E−04 | 1.04E−03 | 0.185 |
| 13 | sCD163 | Q86VB7 | −0.453 | 9.30E−04 | 1.04E−03 | 0.185 |
| 14 | TXD11 | Q6PKC3 | −0.453 | 9.30E−04 | 1.04E−03 | 0.185 |
| 15 | SCGF- | Q9Y240 | −0.453 | 1.00E−03 | 1.05E−03 | 0.185 |
| | alpha | | | | | |
| 16 | Amino- | Q9Y646 | 0.453 | 1.00E−03 | 1.05E−03 | 0.185 |
| | peptidase | | | | | |
| 17 | NID2 | Q14112 | −0.438 | 1.60E−03 | 1.73E−03 | 0.255 |
| 18 | Periostin | Q15063 | −0.438 | 1.60E−03 | 1.73E−03 | 0.255 |
| 19 | USE1 | Q9NZ43 | 0.432 | 2.10E−03 | 2.11E−03 | 0.307 |
| 20 | Angio- | Q15389 | −0.431 | 2.10E−03 | 2.14E−03 | 0.307 |
| | poietin-1 | | | | | |
| 21 | CRIP2 | P52943 | −0.423 | 2.50E−03 | 2.76E−03 | 0.351 |
| 22 | DLC8 | P63167 | −0.422 | 2.60E−03 | 2.85E−03 | 0.351 |
| 23 | SCGF- | Q9Y240 | −0.421 | 2.90E−03 | 2.94E−03 | 0.351 |
| | beta | | | | | |
| 24 | RETST | Q6NUM9 | −0.421 | 2.90E−03 | 2.94E−03 | 0.351 |
| 25 | SIRT2 | Q8IXJ6 | 0.411 | 3.80E−03 | 3.95E−03 | 0.381 |

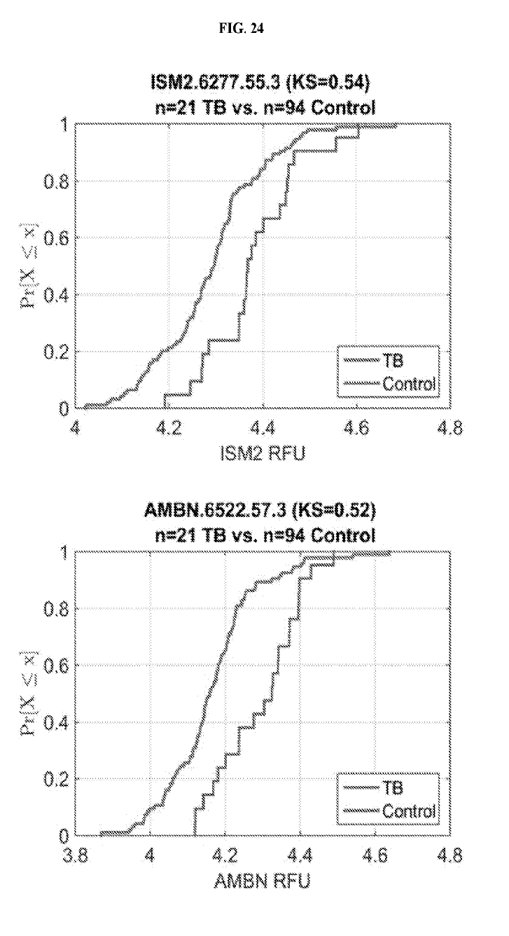

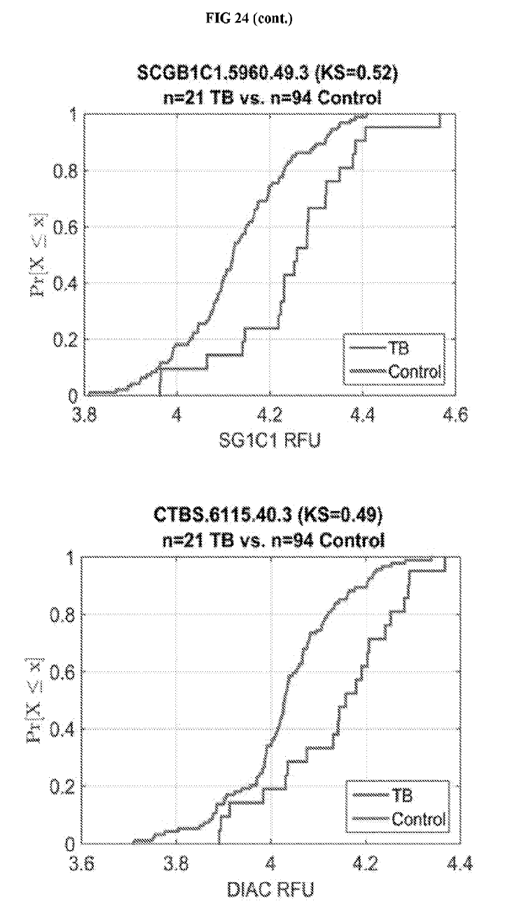

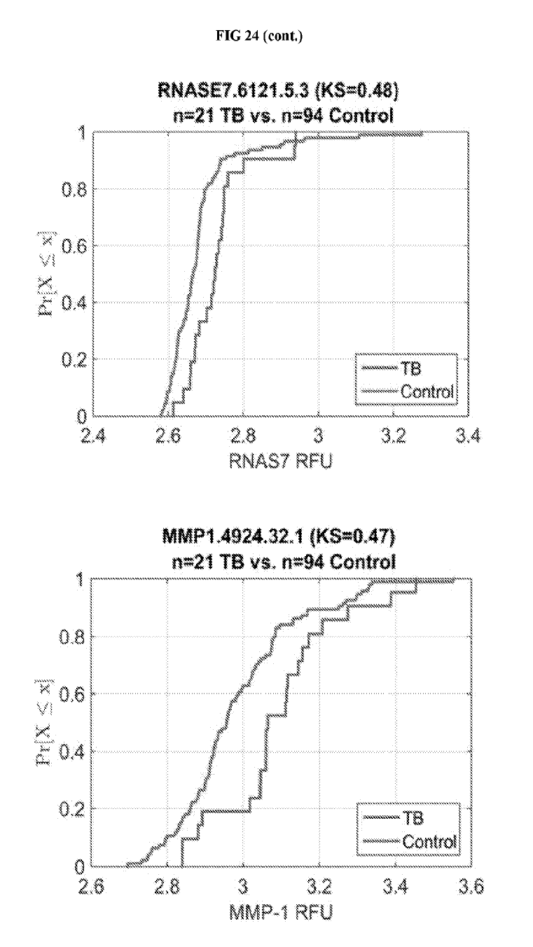

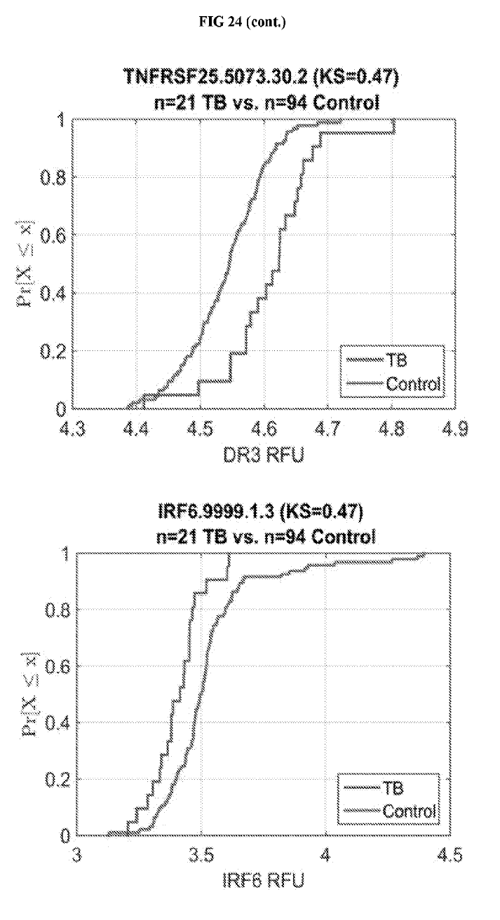

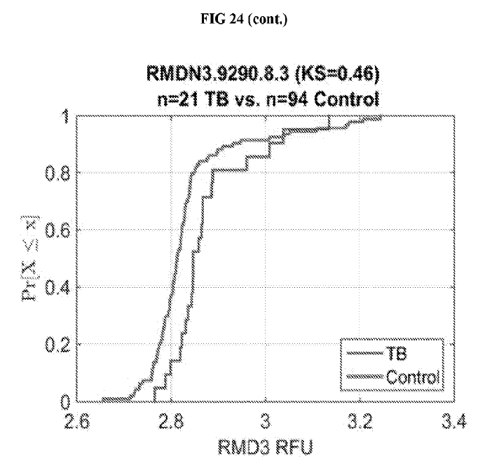

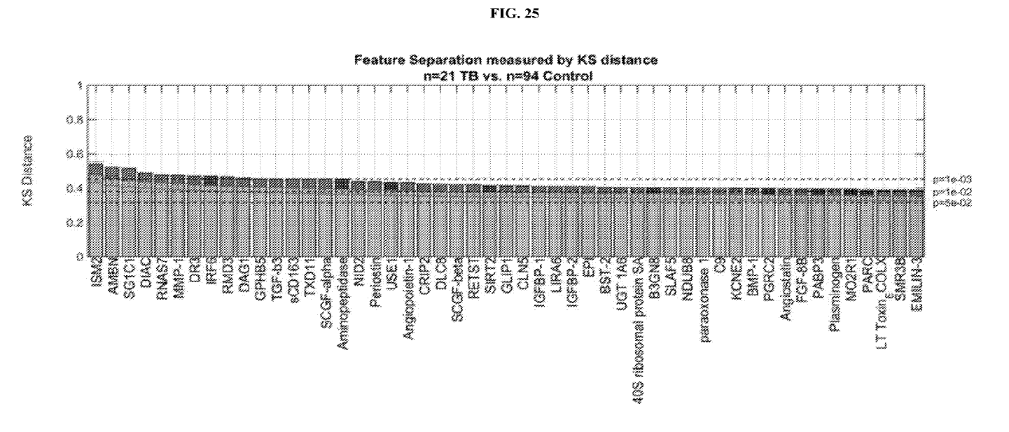

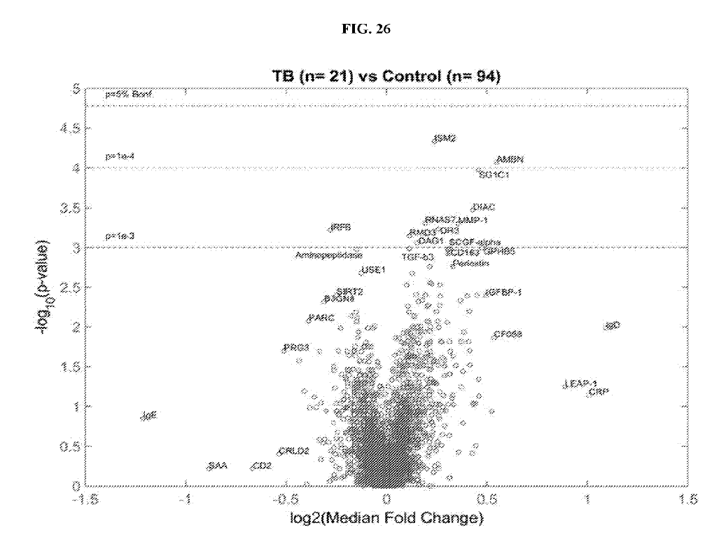

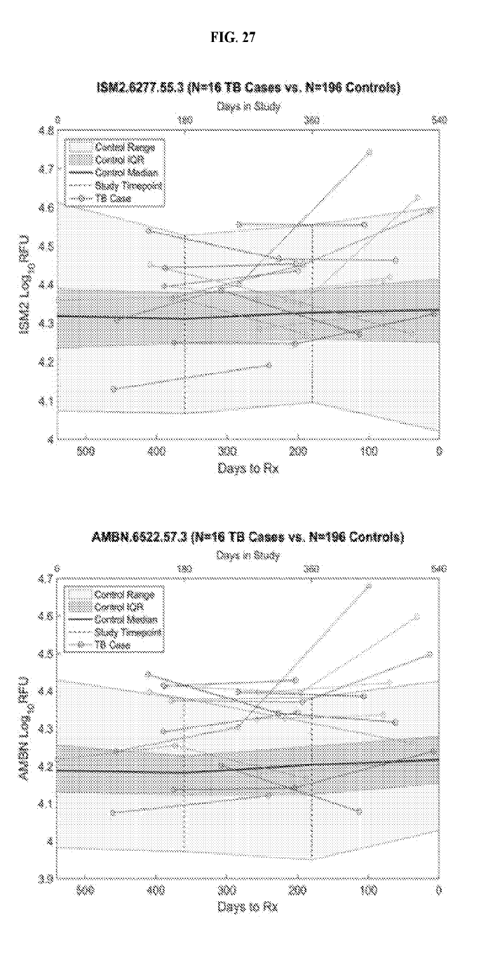

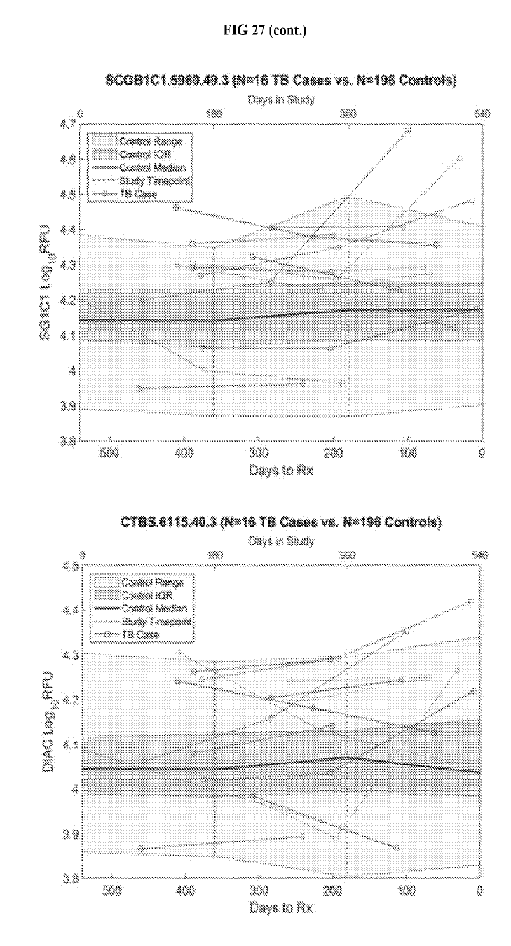

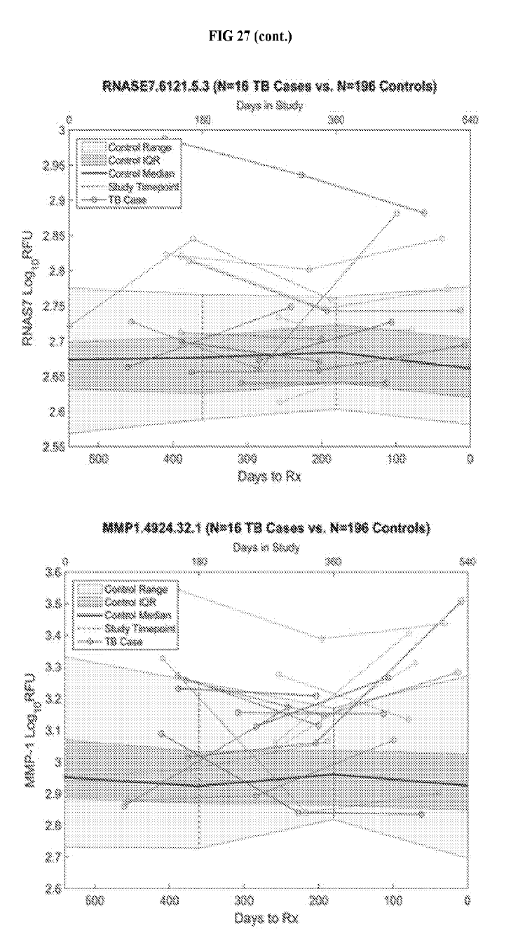

FIG. 24 shows CDFs for the top 9 ranked proteins.

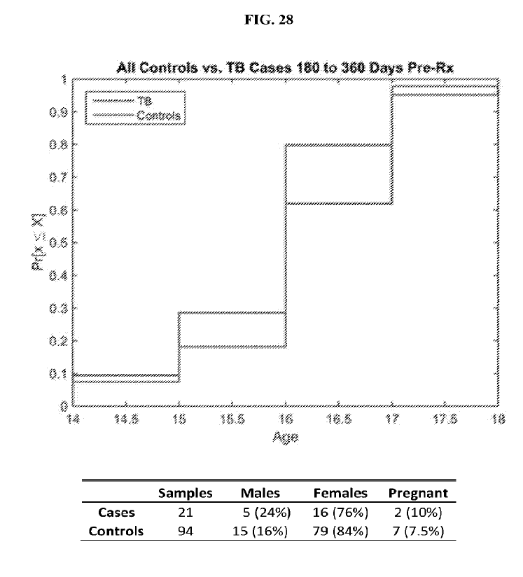

FIG. 28 shows demographics for TB Cases 360 to 540 days pre-treatment along with their matched controls. Comparing 13 TB Cases to 66 matched non-TB Controls, 2 proteins were found to be significant at a 5% FDR. At a 20% FDR 40 proteins were significant with 39 proteins having higher expression in TB cases. Only SIRT2 (p=2e-4, rank 6) was found to be lower. No TB-specific or HR9 proteins were significant at a 20% FDR. Even though the case and control sample sizes were both approximately ⅓ of the 180-360 day time point the magnitude of the differences was noticeably higher. 11 proteins have an FDR less than 10% compared to 3 proteins in the previous time point. Only one protein, RNAS7, was found to be significant in both groups at a 20%, suggesting that the differences between groups may be driven by different biological processes.

TABLE 8
Top 25 ranked proteins differentiating TB Cases 360-540 days pre- treatment from matched
non-TB Controls. Proteins with positive KS distances are lower in TB cases.

| | Target | UniProt | KS | | | |
|---|---|---|---|---|---|---|
| Rank | Name | ID | Dist. | p-value | pₑₘₚ | bhFDR |
| 1 | Cadherin-2 | P19022 | −0.679 | 1.60E−05 | 3.39E−05 | 0.048 |
| 2 | FA20A | Q96MK3 | −0.664 | 3.40E−05 | 5.51E−05 | 0.050 |
| 3 | PIM1 | P11309 | −0.648 | 6.60E−05 | 9.17E−05 | 0.065 |
| 4 | RNAS7 | Q9H1E1 | −0.634 | 1.10E−04 | 1.40E−04 | 0.066 |
| 5 | STX1B | P61266 | −0.634 | 1.10E−04 | 1.40E−04 | 0.066 |
| 6 | SIRT2 | Q8IXJ6 | 0.619 | 2.00E−04 | 2.21E−04 | 0.083 |
| 7 | MA2B2 | Q9Y2E5 | −0.619 | 2.00E−04 | 2.21E−04 | 0.083 |
| 8 | DSC3 | Q14574 | −0.605 | 2.70E−04 | 3.32E−04 | 0.089 |
| 9 | CLC6A | Q6EIG7 | −0.605 | 2.70E−04 | 3.32E−04 | 0.089 |
| 10 | SLUG | O43623 | −0.604 | 3.10E−04 | 3.43E−04 | 0.091 |
| 11 | sperm acrosome associated 5 | Q96QH8 | −0.603 | 3.40E−04 | 3.55E−04 | 0.091 |
| 12 | ELA2A | P08217 | −0.589 | 5.10E−04 | 5.27E−04 | 0.124 |
| 13 | NRN1 | Q9NPD7 | −0.587 | 5.60E−04 | 5.44E−04 | 0.126 |
| 14 | GALT1 | Q10472 | −0.575 | 7.00E−04 | 7.76E−04 | 0.146 |
| 15 | TPA_MOUSE | P11214 | −0.573 | 8.00E−04 | 8.01E−04 | 0.155 |
| 16 | EMIL3 | Q9NT22 | −0.572 | 8.60E−04 | 8.27E−04 | 0.157 |
| 17 | transcription factor MLR1, . . . | Q8N3X6 | −0.559 | 1.10E−03 | 1.17E−03 | 0.183 |
| 18 | SC61B | P60468 | −0.558 | 1.10E−03 | 1.20E−03 | 0.183 |
| 19 | C5b, 6 Complex | P01031, P13671 | −0.557 | 1.30E−03 | 1.24E−03 | 0.183 |
| 20 | Galectin-7 | P47929 | −0.545 | 1.40E−03 | 1.69E−03 | 0.183 |
| 21 | C5 | P01031 | −0.544 | 1.60E−03 | 1.74E−03 | 0.183 |
| 22 | sCD4 | P01730 | −0.544 | 1.60E−03 | 1.74E−03 | 0.183 |
| 23 | IL-23 | P29460, Q9NPF7 | −0.544 | 1.60E−03 | 1.74E−03 | 0.183 |
| 24 | NLGN2 | Q8NFZ4 | −0.544 | 1.60E−03 | 1.74E−03 | 0.183 |
| 25 | GLIP1 | P48060 | −0.544 | 1.60E−03 | 1.74E−03 | 0.183 |

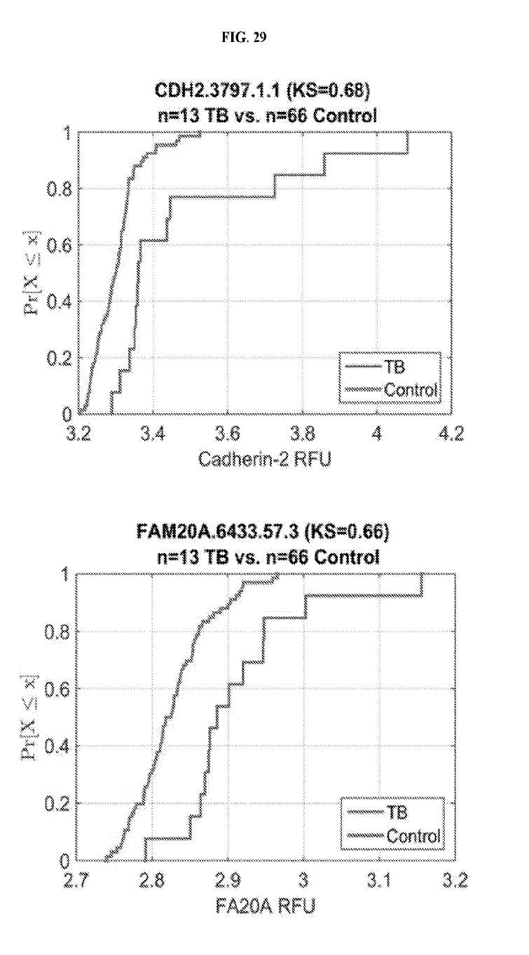

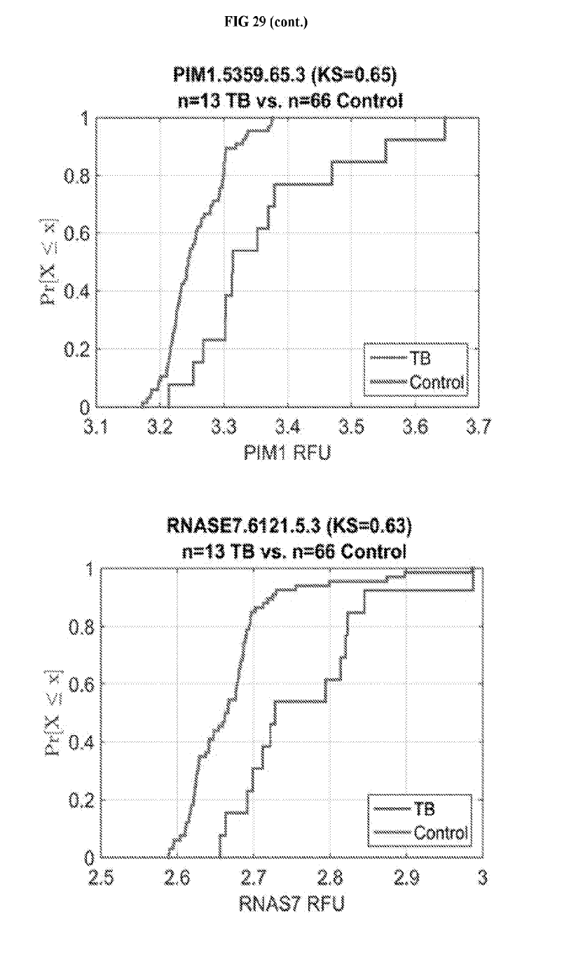

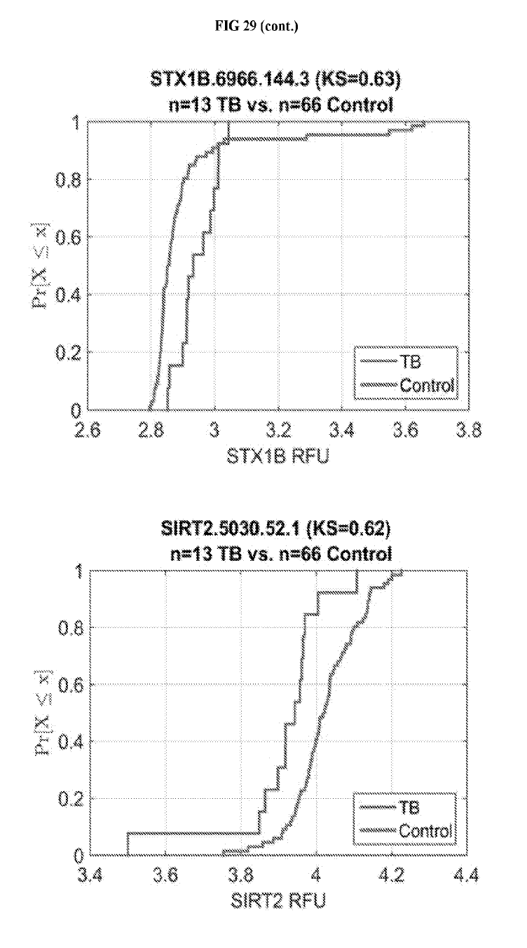

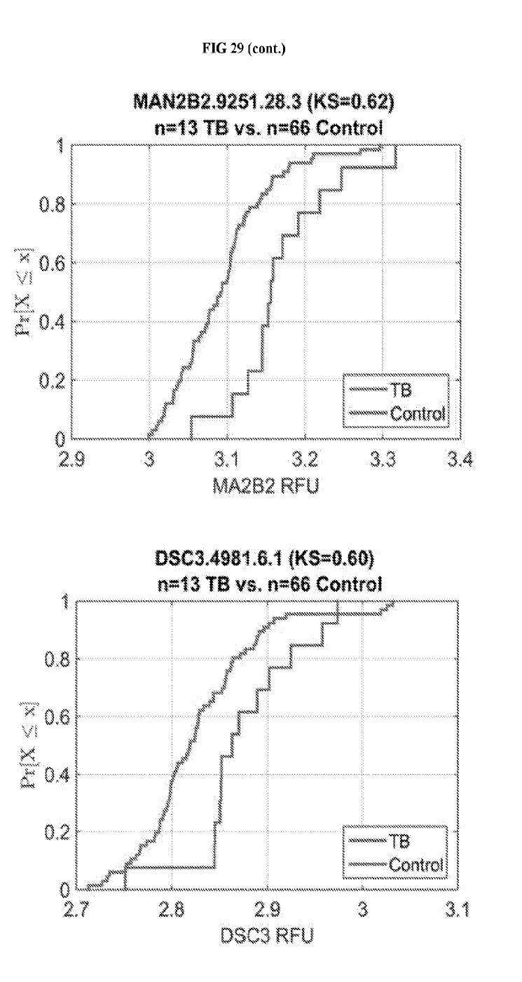

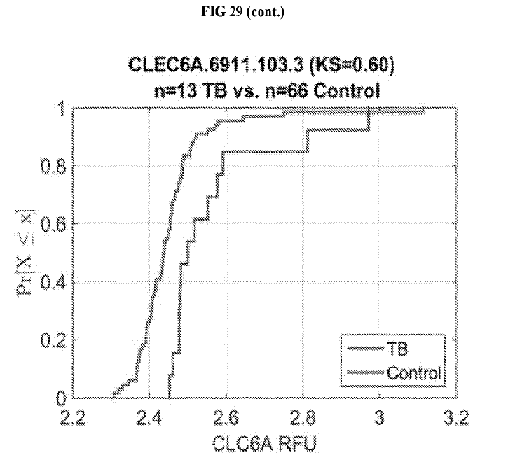

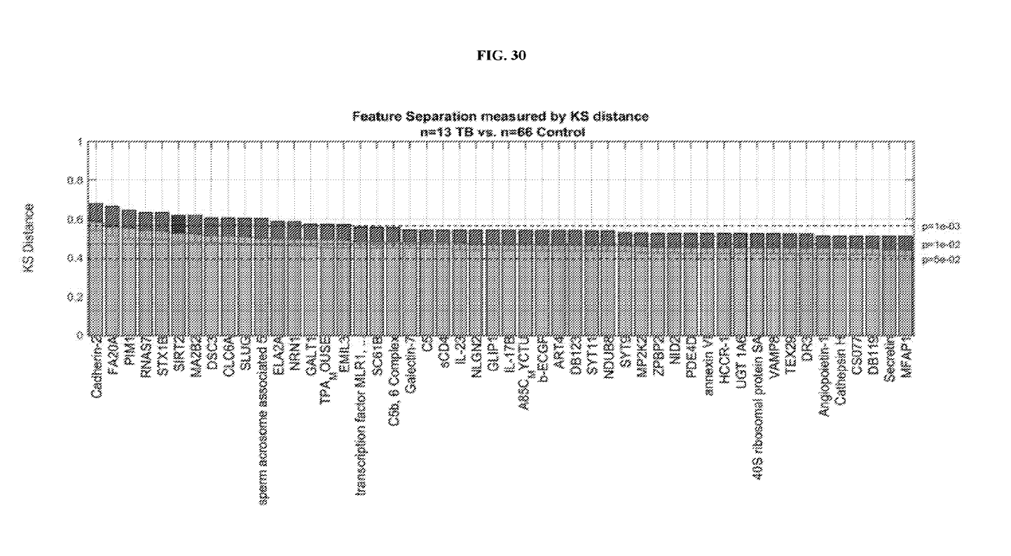

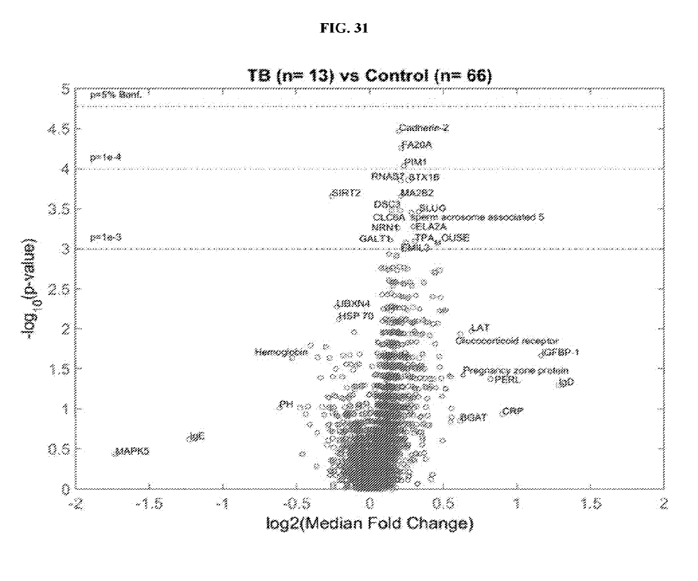

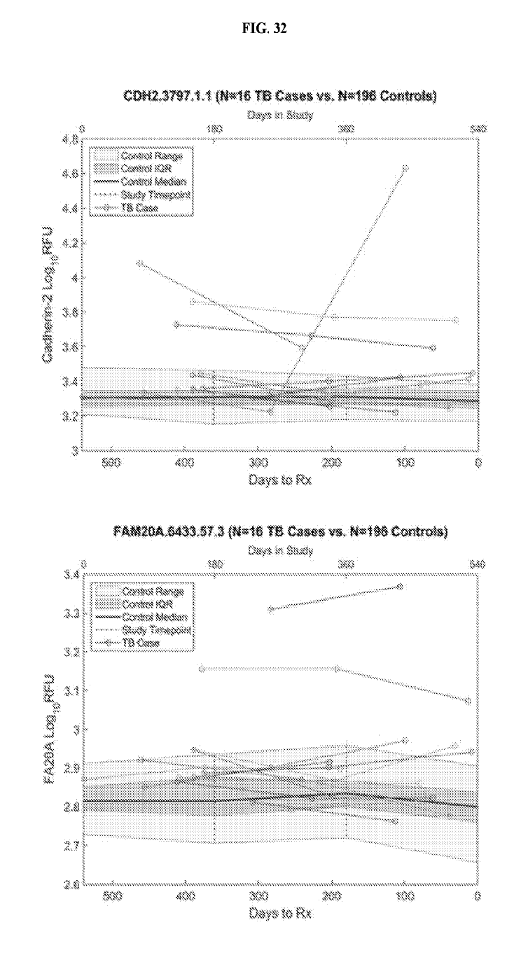

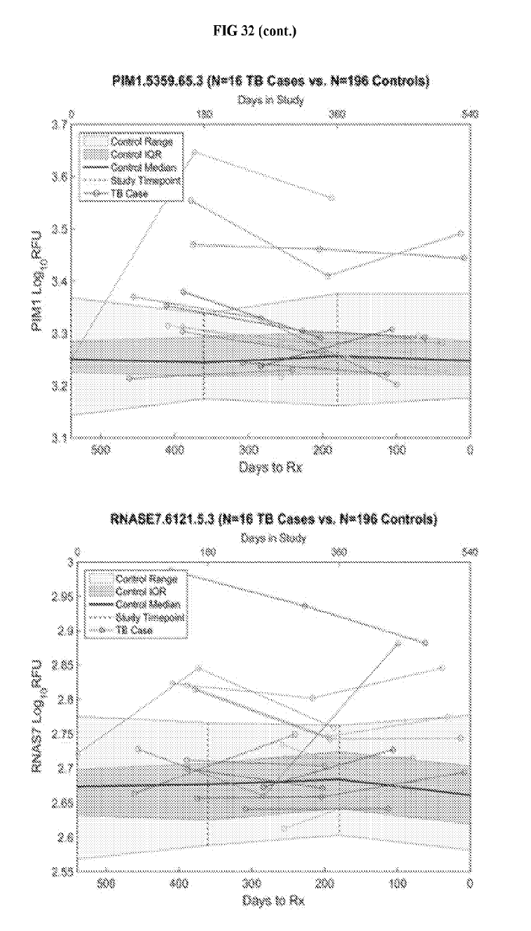

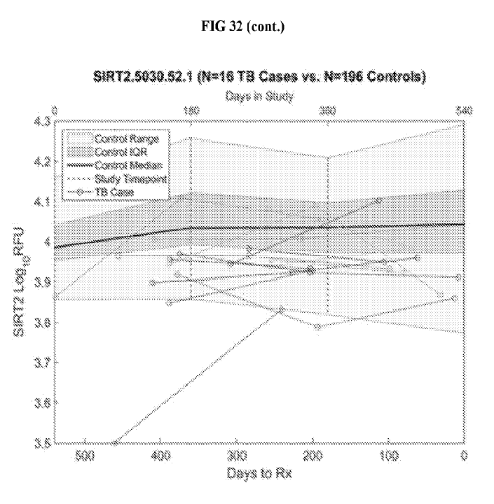

FIGS. 29-32 show CDFs of the top 9, the top 100 features with class randomization statistics, a volcano plot and control band plots for the top 6, respectively.

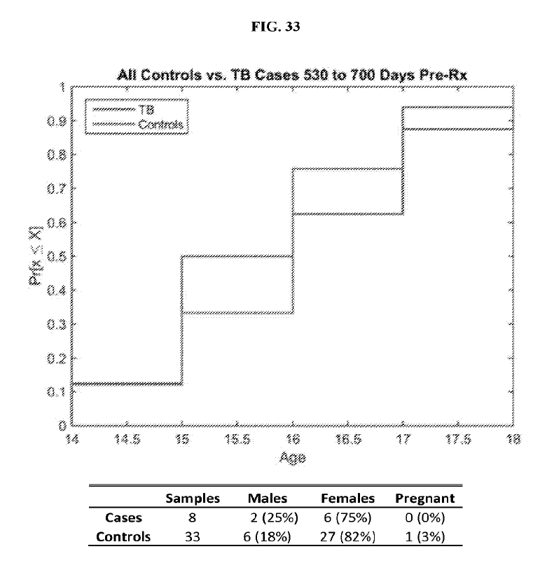

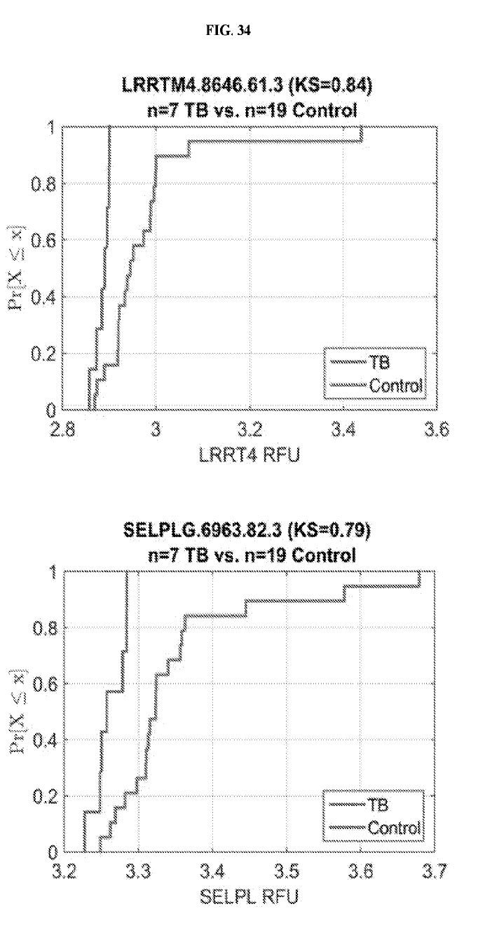

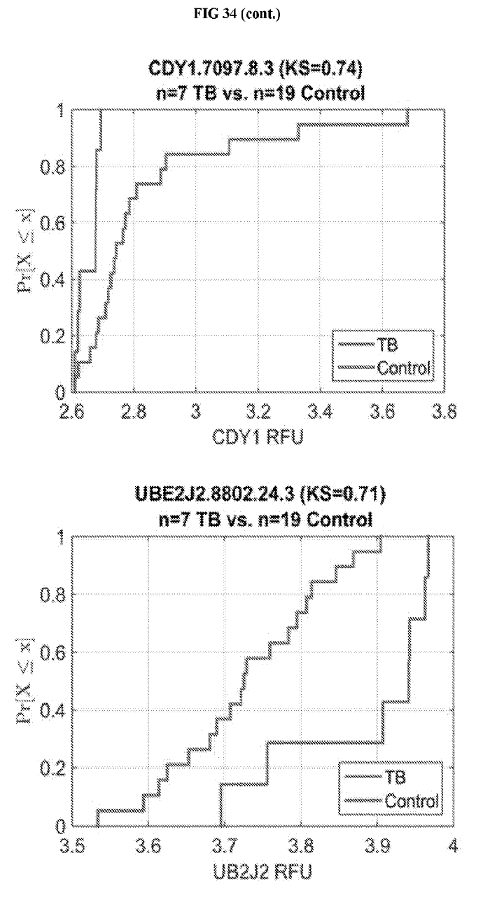

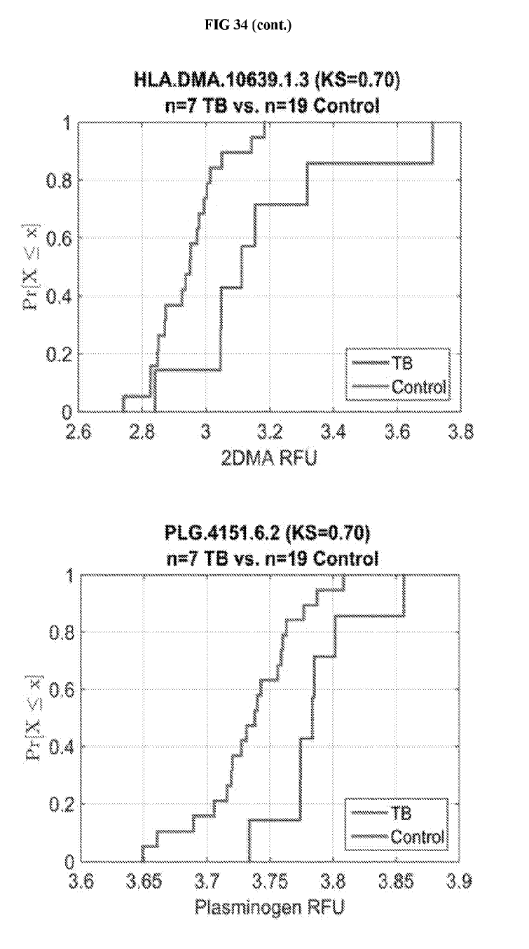

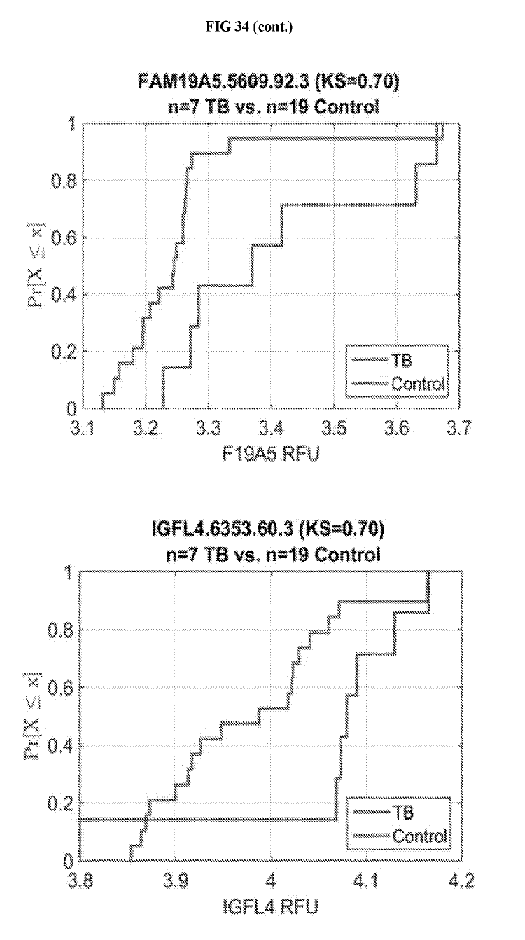

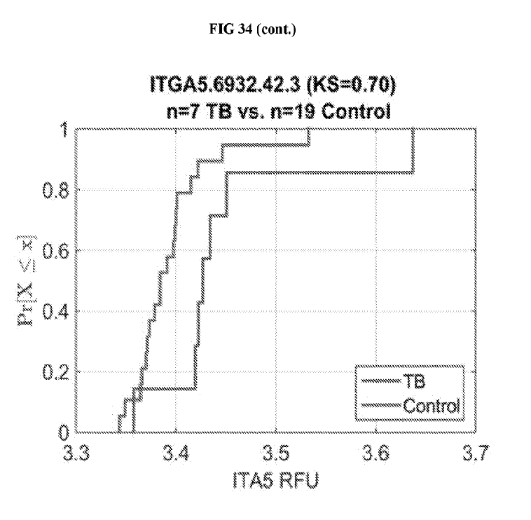

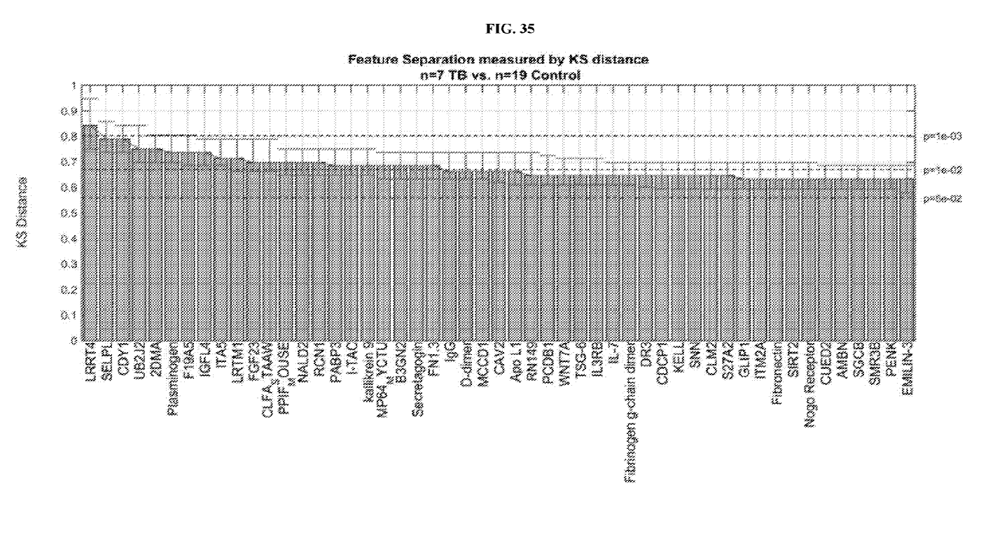

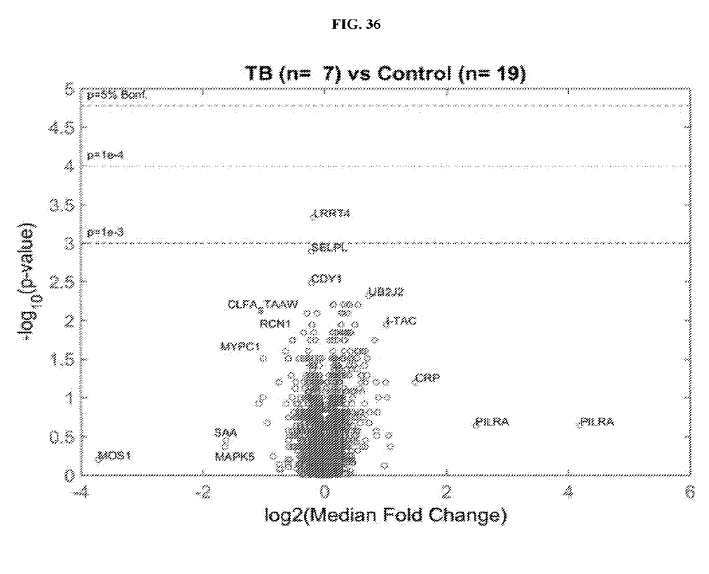

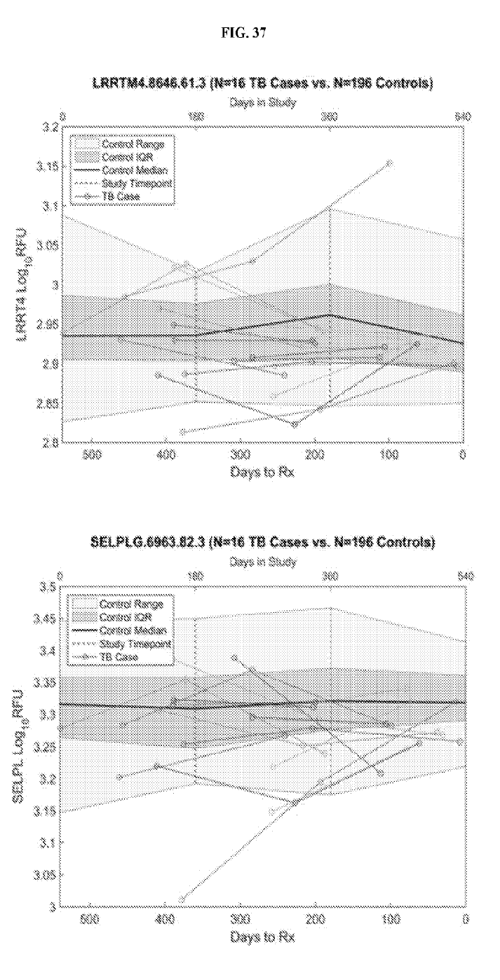

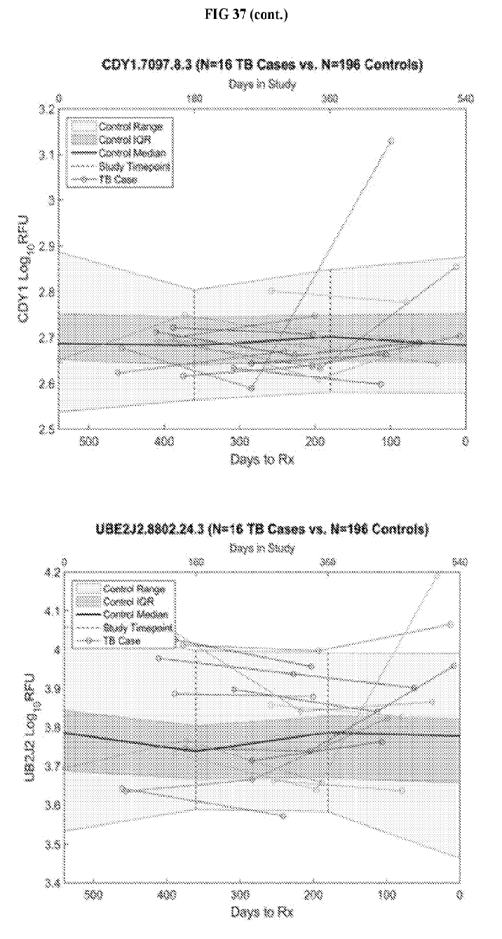

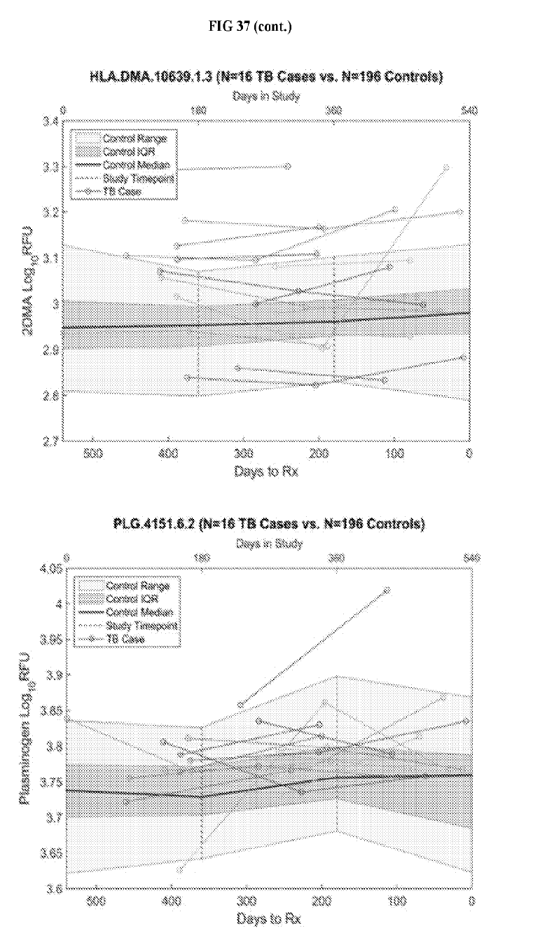

FIG. 33 shows demographics for TB cases 540 to 700 days pre-treatment, as well as their matched controls. Comparing 8 TB Cases with 33 matched non-TB Controls, 0.92 was the smallest FDR attained. However, the KS distances were relatively large with an absolute range of [0.842 0.699] for the top 10 ranked proteins. Table 9 below shows KS statistics for the top 25 proteins. Plasminogen (#6), I-TAC (#17), Fibronectin (#22), D-dimer (#24). IgG (#22) and D-dimer (#7) were also found to be a top 20 markers in the 0-180 time point. Also, 2DMA (#5) is a major histo-compatibility antigen which has implications in infection. FIGS. 34-37 shows biomarker data for 540 to 700 days pre-treatment.

TABLE 9
Top 25 ranked proteins differentiating TB Cases 540-700 days pre- treatment from matched
non-TB Controls. Proteins with positive KS distances are lower in TB cases.

| | Target | UniProt | KS | | | |
|---|---|---|---|---|---|---|
| Rank | Name | ID | Dist. | p-value | pₑₘₚ | bhFDR |
| 1 | LRRT4 | Q86VH4 | 0.842 | 4.30E−04 | 4.60E−04 | 0.922 |
| 2 | SELPL | Q14242 | 0.789 | 1.30E−03 | 1.27E−03 | 0.922 |
| 3 | CDY1 | Q9Y6F8 | 0.737 | 2.90E−03 | 3.28E−03 | 0.922 |
| 4 | UB2J2 | Q8N2K1 | −0.714 | 4.00E−03 | 4.82E−03 | 0.922 |
| 5 | 2DMA | P28067 | −0.699 | 6.30E−03 | 6.20E−03 | 0.922 |
| 6 | Plasminogen | P00747 | −0.699 | 6.30E−03 | 6.20E−03 | 0.922 |
| 7 | F19A5 | Q7Z5A7 | −0.699 | 6.30E−03 | 6.20E−03 | 0.922 |
| 8 | IGFL4 | Q6B9Z1 | −0.699 | 6.30E−03 | 6.20E−03 | 0.922 |
| 9 | ITA5 | P08648 | −0.699 | 6.30E−03 | 6.20E−03 | 0.922 |
| 10 | LRTM1 | Q9HBL6 | −0.699 | 6.30E−03 | 6.20E−03 | 0.922 |
| 11 | FGF23 | Q9GZV9 | −0.684 | 8.80E−03 | 7.93E−03 | 0.922 |
| 12 | CLFA_STAAW | Q8NXJ1 | 0.684 | 8.80E−03 | 7.93E−03 | 0.922 |
| 13 | PPIF_MOUSE | Q99KR7 | −0.684 | 8.80E−03 | 7.93E−03 | 0.922 |
| 14 | NALD2 | Q9Y3Q0 | −0.684 | 8.80E−03 | 7.93E−03 | 0.922 |
| 15 | RCN1 | Q15293 | 0.684 | 8.80E−03 | 7.93E−03 | 0.922 |
| 16 | PABP3 | Q9H361 | 0.662 | 1.10E−02 | 1.13E−02 | 0.922 |
| 17 | I-TAC | O14625 | −0.662 | 1.10E−02 | 1.13E−02 | 0.922 |
| 18 | kallikrein 9 | Q9UKQ9 | 0.662 | 1.10E−02 | 1.13E−02 | 0.922 |
| 19 | MP64_MYCTU | P0A5Q4 | −0.662 | 1.10E−02 | 1.13E−02 | 0.922 |
| 20 | B3GN2 | Q9NY97 | −0.662 | 1.10E−02 | 1.13E−02 | 0.922 |
| 21 | Secretagogin | O76038 | −0.647 | 1.30E−02 | 1.43E−02 | 0.922 |
| 22 | FN1.3 | P02751 | −0.647 | 1.30E−02 | 1.43E−02 | 0.922 |
| 23 | IgG | P01857 | −0.647 | 1.30E−02 | 1.43E−02 | 0.922 |
| 24 | D-dimer | P02671 P02675 P02679 | −0.647 | 1.30E−02 | 1.43E−02 | 0.922 |
| 25 | MCCD1 | P59942 | −0.647 | 1.30E−02 | 1.43E−02 | 0.922 |

### Stability Selection using Logistic Regression

Metadata for GENDER, SITE_ID, BMI, and AGE were included along with all 3040 human, non-human, and TB-specific proteins when performing stability selection using an L1-regularized logistic regression model. As with the univariate KS analysis this was done using all TB cases and matched controls, then within each time point.

Outlying values in logistic regression affect the hyperplane and can greatly influence the learning procedure. Initial runs of stability selection found that the L1-regularized model was very sensitive to a small subset of outliers (1-3 TB cases) and overly selecting for these proteins. Outliers were defined as being >4 median absolute deviations (MADs) from the median and were replaced with values taken from the 90% percentile of a simulated distribution.

Table 10 below shows stability selection statistics for proteins with maximum selection probabilities exceeding 50% when comparing all TB Case samples with their matched Control samples.

TABLE 3
Stability selection statistics for proteins
with a maximum selection probability >50%.

| Rank | Target | max(Pr{Selection}) | Area |
|---|---|---|---|
| 1) | SIRT2 | 0.858 | 0.10 |
| 2) | AMBN | 0.795 | 0.10 |
| 3) | C5 | 0.777 | 0.11 |
| 4) | B3GN8 | 0.748 | 0.06 |
| 5) | LD78-beta | 0.700 | 0.05 |
| 6) | MMP-1 | 0.683 | 0.05 |
| 7) | KI2LA | 0.608 | 0.03 |
| 8) | PCD10 | 0.592 | 0.04 |
| 9) | IL-7 | 0.570 | 0.03 |
| 10) | CXCL16, soluble | 0.538 | 0.05 |
| 11) | DR3 | 0.522 | 0.07 |

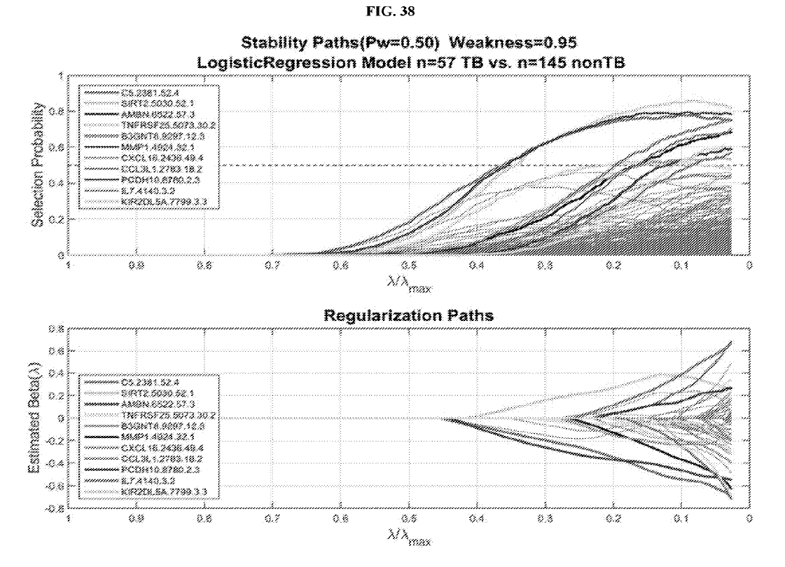

AMBN, C5, MMP-1, DR3, SIRT2, and C9 were also found to be in the top 25 proteins ranked by KS distance comparing all TB Case to all Control samples. FIG. 38 shows the stability paths for all proteins in the upper panel and the regularization paths in the bottom. Stability paths are labeled by total area under the path in each figure, while tables are ranked by maximum selection probability.

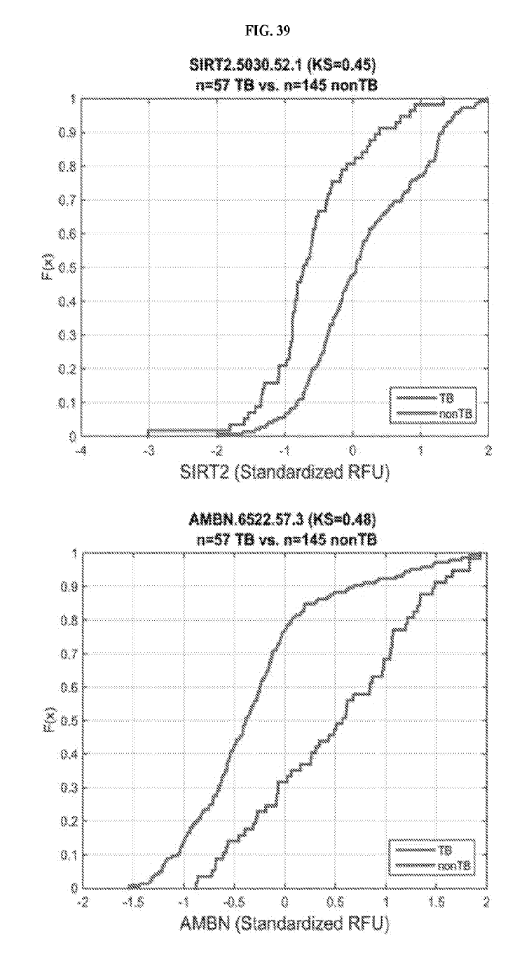

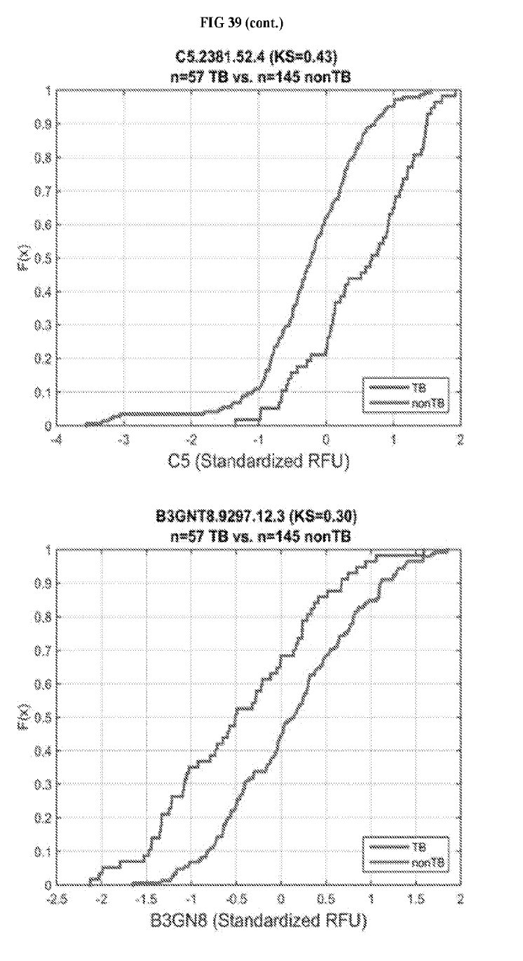

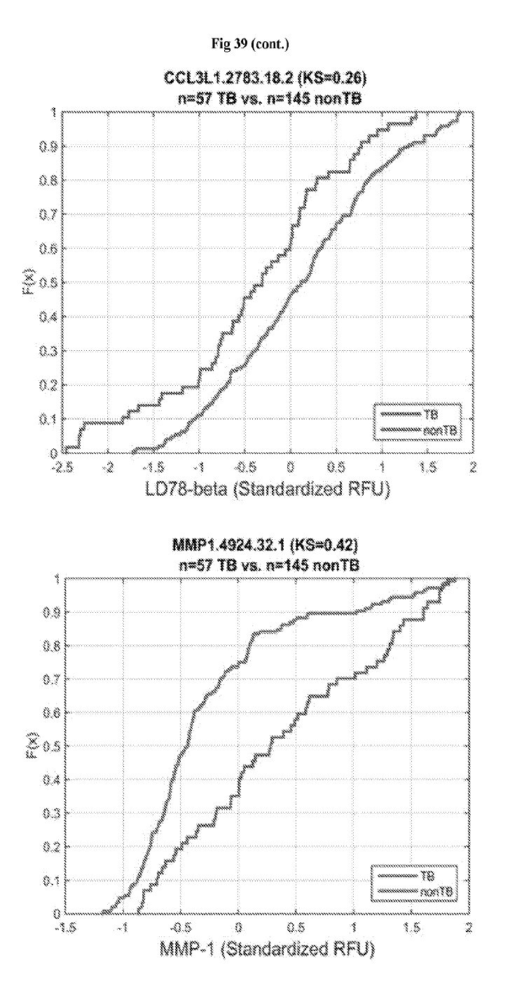

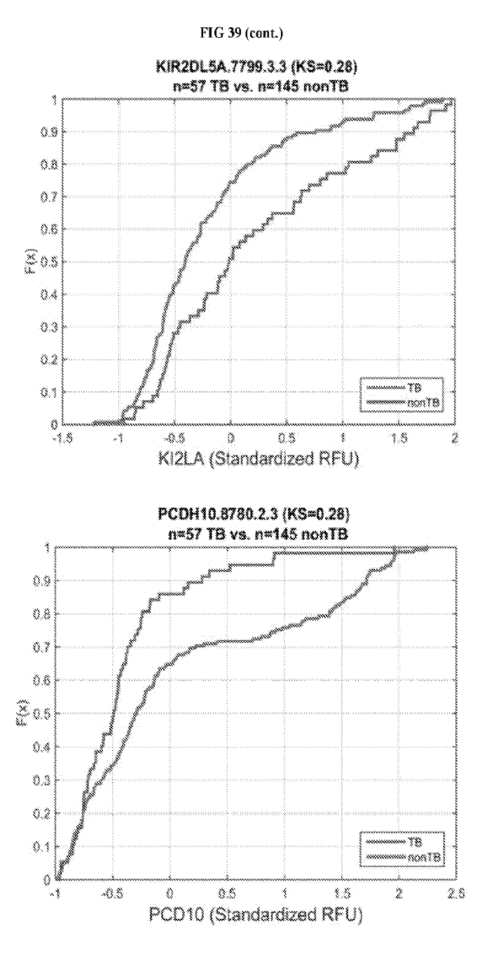

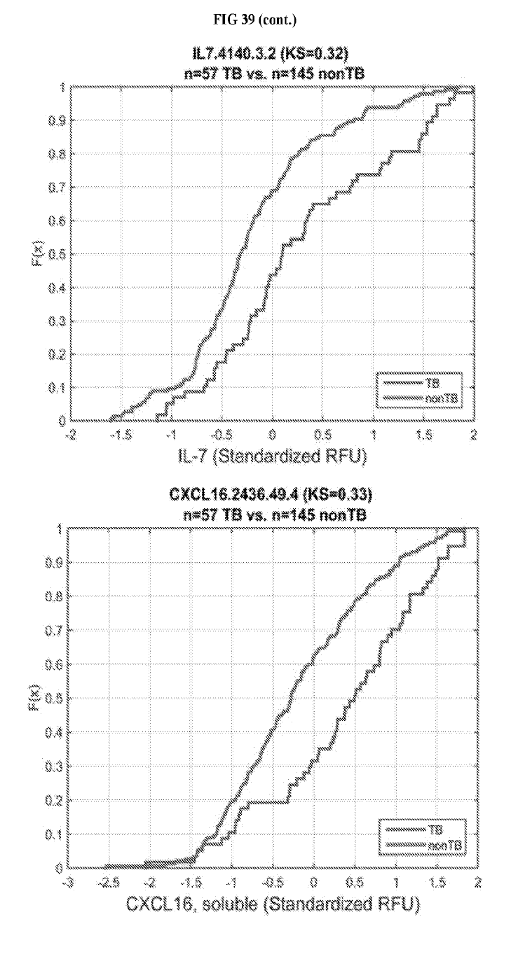

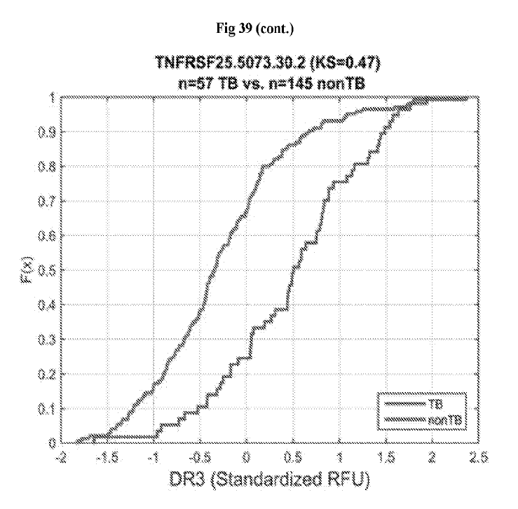

FIG. 39 shows CDFs for the top 6 proteins ranked by selection probability. Logistic regression was performed on standardized RFU values |(X−μ)/σ| where μ is the global mean, which are the units of the CDFs and can be interpreted as number of standard deviations from the mean. Although values |(X−μ)/σ|>4 were replaced with simulated values from the 90th percentile of a simulated distribution, the CDF plots show the actual standardized scores without replacement. No proteins in the top 11 were observed to have values |(X−μ)/σ|>4, suggesting outliers were indeed driving the selection of the proteins when not removed.

### Example 3

### Model Building

The objective of the ‘responsive’ model is to identify Progressors 6-12 months prior to treatment with reasonable accuracy and Progressors 0-6 months prior to treatment with a high accuracy, while maintaining a near monotonic change in log-odds. Due to the uncertainty surrounding the actual date of diagnosis and a relatively small number of subjects with repeated measures, longitudinal models are fit and assessed using data binned by time compared to matched controls.

The objectives of the model are met by features which distinguish Progressors from Controls 6-12 months before diagnosis and maintain a near monotonic increase in binned RFU from 540 days pre-treatment to the date of treatment.

These features were selected by ranking proteins according to the KS distance separating Progressors (n=21) from matched Controls (n=94) 180-360 days pre-treatment. The responsiveness of highly significant proteins (FDR<0.05) was then tested using the Mack Wolfe Test. Also called the umbrella test, it is a k-sample problem which tests for the form F₁(x)≥ . . . ≥Fᵢ(x)≤ . . . ≤Fₖ(x), or vice versa, where a monotonic trend is seen to the sample group Fᵢ where the trend reverses. In the current data, a monotonic trend was specified for 540+ to 0-90 days post-treatment, then a reversal for the sample group at 90-180 days post-treatment.

A KS distance of 0.35 was used, which corresponds to a qvalue of 0.07. All features were then run through the umbrella ranker with the 0-90 day time point after treatment as the peak. This was done using increasing and decreasing assumptions. A q-value of 0.05 was used as a threshold and this resulted in a list of 37 features. These features were investigated for differences in class medians in the 0-360 day time period. Any values less than 250 were discarded, as were CV>20. CRP and Albumin were discarded due to their lack of specificity. Table 11 shows the resulting list of 23 proteins.

TABLE 11
List of features that show responsiveness to time as well
as diagnostic performance 180-360 days before diagnosis

| | | | KS Test | | | Mack-Wolfe Umbrella Test | | | | | |
|---|---|---|---|---|---|---|---|---|---|---|---|
| | Target | UniProt | Rank | Dist | qval | Rank | Stat | pval | qval | Δ(Class) | CV (%) |
| 1 | MMP-1 | ‘P03956’ | 5 | 0.48 | 0.01 | 47 | 2.61 | 4.57E−03 | 0.3 | 395.5 | 17.7 |
| 2 | C9 | ‘P02748’ | 6 | 0.48 | 0.01 | 18 | 3.1 | 9.70E−04 | 0.16 | 3903 | 5.9 |
| 3 | D-dimer | ‘P02671’ | 14 | 0.44 | 0.01 | 58 | 2.5 | 6.21E−03 | 0.32 | 3499.2 | 5.72 |
| 4 | IP-10 | ‘P02778’ | 24 | 0.42 | 0.02 | 10 | 3.43 | 3.00E−04 | 0.09 | 412.1 | 5.08 |
| 5 | IGFBP-2 | ‘P18065’ | 64 | 0.39 | 0.03 | 6 | 3.59 | 1.64E−04 | 0.08 | 2468.6 | 2.53 |
| 6 | MIG | ‘Q07325’ | 81 | 0.37 | 0.05 | 28 | 2.91 | 1.83E−03 | 0.2 | 286.9 | 5.21 |
| 7 | NPS | ‘P0C0P6’ | 16 | 0.44 | 0.01 | 3001 | 4 | 1.00E+00 | 1 | −2238 | 5.69 |
| 8 | CA2D3 | ‘Q8IZS8’ | 17 | 0.44 | 0.01 | 1 | −5.3 | 5.64E−08 | 0 | −967.5 | 7.24 |
| 9 | MMP-2 | ‘P08253’ | 20 | 0.43 | 0.02 | 210 | −2.78 | 2.73E−03 | 0.04 | −1454.7 | 5.68 |
| 10 | CD79A | ‘P11912’ | 21 | 0.43 | 0.02 | 126 | −3.2 | 6.98E−04 | 0.02 | −362.7 | 1.74 |
| 11 | PKB a/b/g | ‘Family’ | 26 | 0.42 | 0.02 | 255 | −2.64 | 4.16E−03 | 0.05 | −390.1 | 6.63 |
| 12 | PGCB | ‘Q96GW7’ | 36 | 0.41 | 0.03 | 434 | −2.09 | 1.82E−02 | 0.13 | −359.7 | 7.89 |
| 13 | PABP3 | ‘Q9H361’ | 37 | 0.4 | 0.03 | 247 | −2.65 | 4.03E−03 | 0.05 | −797 | 5.06 |
| 14 | MXRA7 | ‘P84157’ | 55 | 0.39 | 0.03 | 67 | −3.67 | 1.23E−04 | 0.01 | −534.4 | 6.01 |
| 15 | CNTFR | ‘P26992’ | 62 | 0.39 | 0.03 | 86 | −3.51 | 2.27E−04 | 0.01 | −1162.9 | 6.13 |
| | alpha | | | | | | | | | | |
| 16 | Nr-CAM | ‘Q92823’ | 67 | 0.38 | 0.04 | 5 | −4.91 | 4.59E−07 | 0 | −3969.2 | 6.00 |
| 17 | Ephrin-A3 | ‘P52797’ | 68 | 0.38 | 0.04 | 77 | −3.6 | 1.58E−04 | 0.01 | −1169.8 | 2.94 |
| 18 | CD36 Ag | ‘P16671’ | 69 | 0.38 | 0.04 | 108 | −3.29 | 4.97E−04 | 0.01 | −2779.3 | 11.3 |
| 19 | NDUB4 | ‘O95168’ | 71 | 0.38 | 0.04 | 394 | −2.19 | 1.43E−02 | 0.11 | −941.8 | 9.76 |
| 20 | PCI | ‘P05154’ | 78 | 0.37 | 0.05 | 81 | −3.56 | 1.86E−04 | 0.01 | −13481.2 | 9.08 |
| 21 | BOC | ‘Q9BWV1’ | 87 | 0.37 | 0.05 | 73 | −3.61 | 1.51E−04 | 0.01 | −891.1 | 6.25 |
| 22 | PKB beta | ‘P31751’ | 88 | 0.37 | 0.05 | 262 | −2.61 | 4.57E−03 | 0.05 | −1226 | 6.77 |
| 23 | JKIP3 | ‘Q5VZ66’ | 92 | 0.37 | 0.05 | 135 | −3.14 | 8.54E−04 | 0.02 | −330.8 | 12.26 |

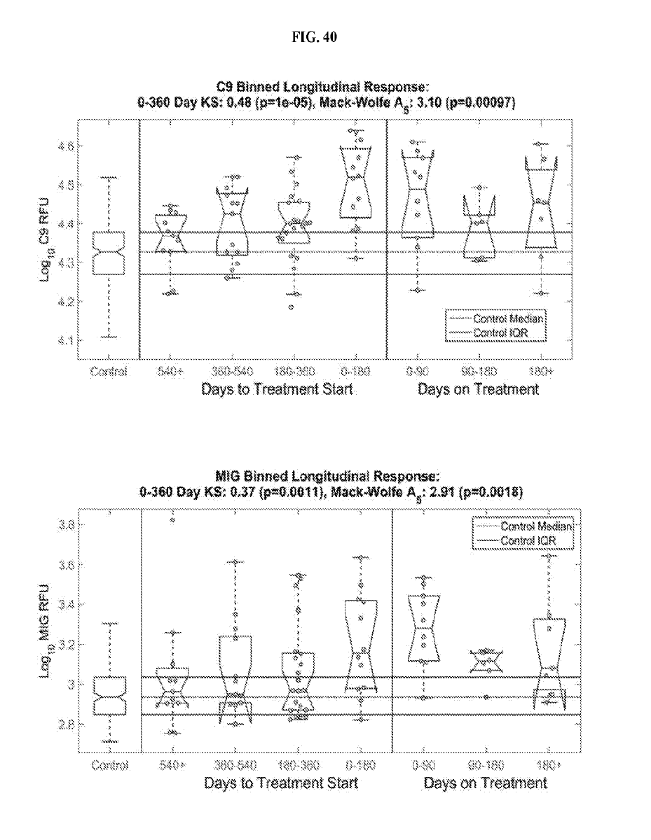

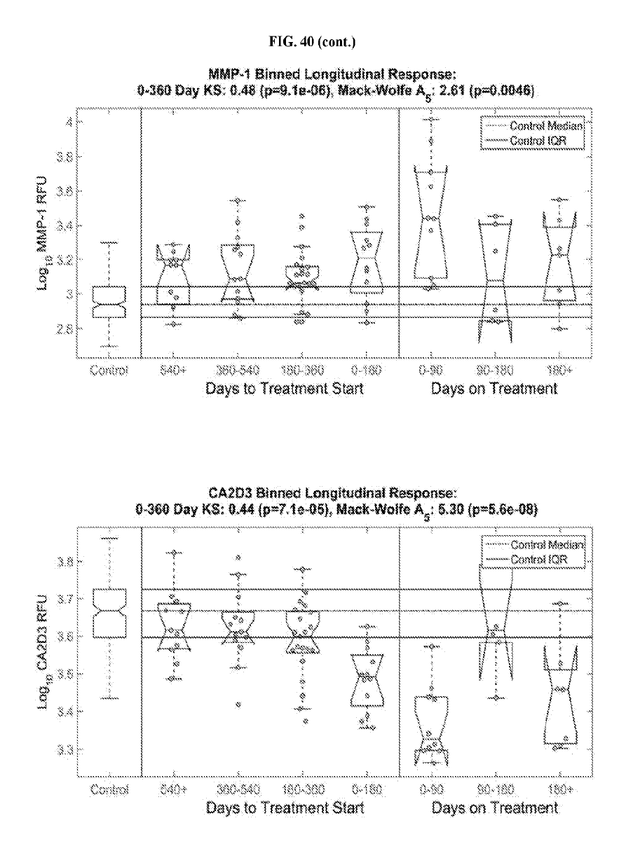

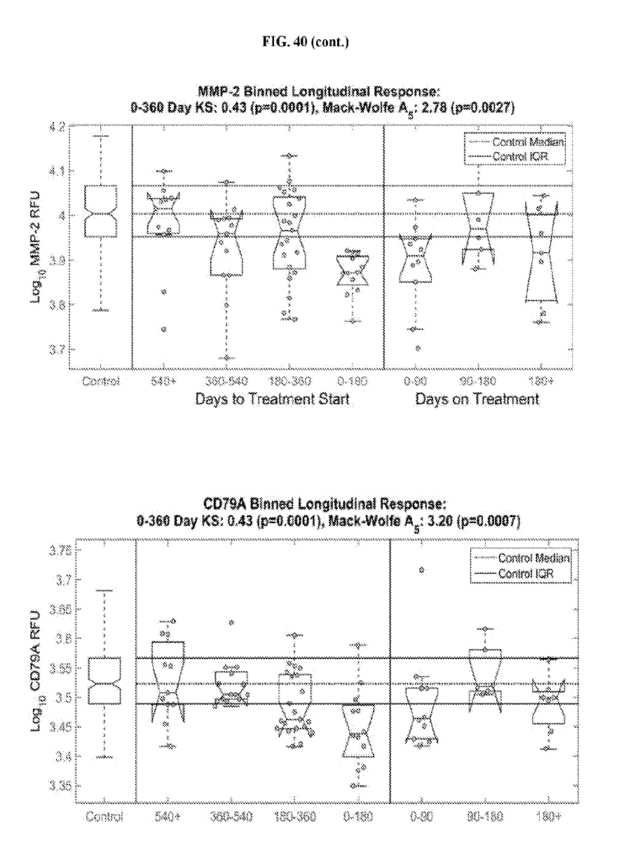

FIG. 40 shows box plots of log10 RFU level binned by time to diagnosis for six example proteins. The median and IQR of the control data has been extended for a reference range of values.

FIG. 41 the univariate KS distances as a function of the Mack-Wolfe statistic. Negative statistics indicate a decreasing trend.

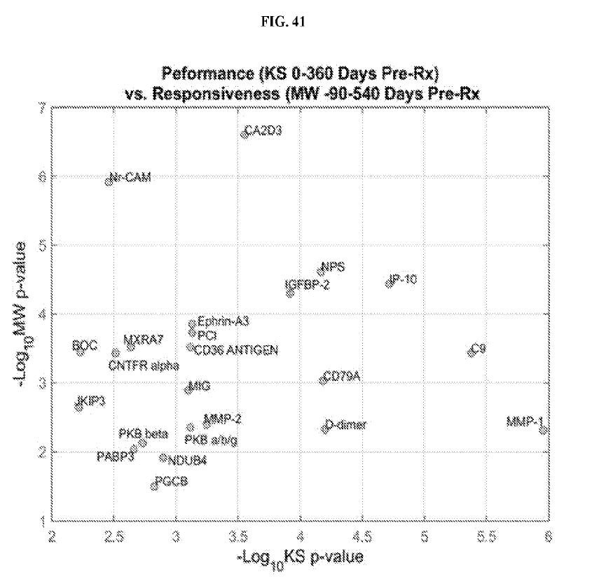

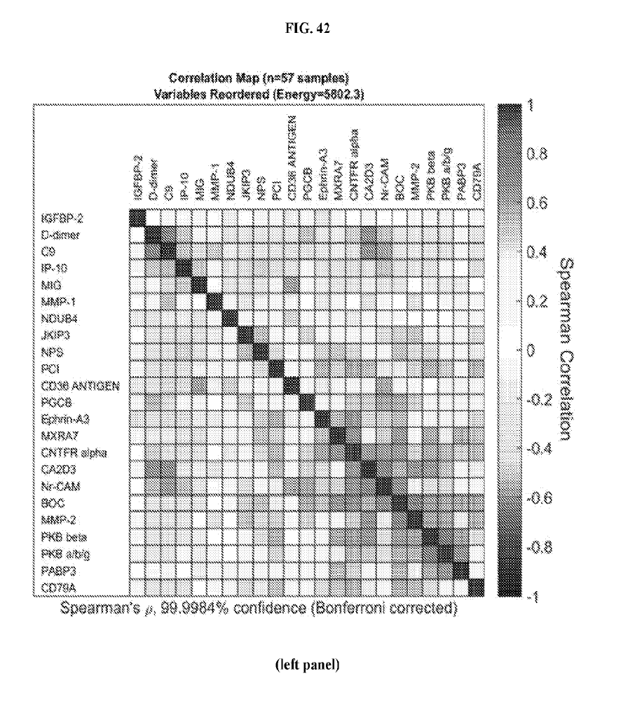

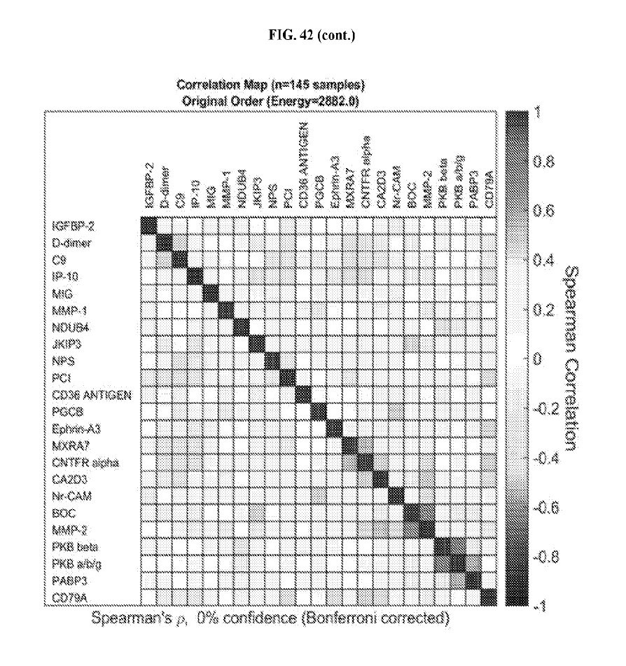

Seriation was performed on the RFU data for the TB and Control data separately to investigate the correlation structure in the responsive feature list. Seriation was first performed on the TB samples, then this structure was imposed upon the Control samples. FIG. 42 shows the correlation maps for the 23 proteins in Table 11 in Progressors (left). The structure from the Progressor matrix was then applied to the Control samples (right).

Since the proteins were selected to show monotonic increases or decreases in RFU level, it would be expected that they show a certain level of correlation. The correlation map on the left of FIG. 42 shows two clusters corresponding to features which increase with time (top left, all with positive MW statistics) and those which decrease (the rest). When this structure is imposed on the Control samples (right panel of FIG. 42) MMP-2/BOC and the PKB-beta/PKB family proteins show a high level of correlation.

Despite the uncertainty in the time to treatment estimates, an attempt was made to leverage the continuous nature of the variable. Loess Fits using a linear local regression span were used with bootstrap 95% confidence intervals (n=1000). The span parameter was either held at 0.5 or cross-validated. The responsiveness and classification performance of each protein was quantified by calculating the area between the lower 95% confidence bound of the Loess fit and the upper IQR of the control population (for a protein increasing towards initiation of treatment, or vice versa). Only distances between the confidence bound and upper IQR were taken into account and therefore all areas were positive. Although simple, this technique takes the temporal variance of the data into account.

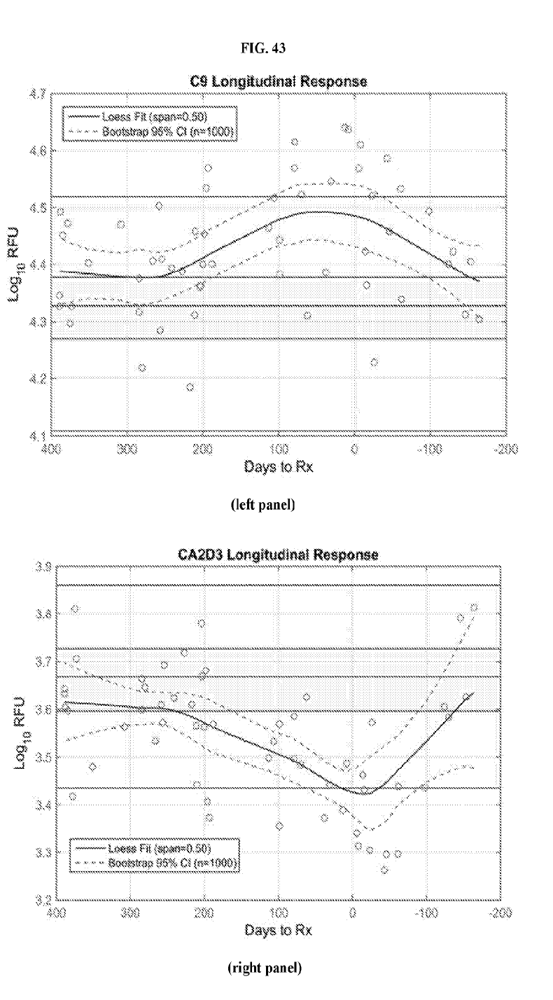

FIG. 43 shows example plots for C9 and CA2D3. The Loess fits and 95% confidence bounds are overlaid onto a standard control band for reference. When ranked by total area, the top 20 proteins were all found to be in Table 11. This indicates that, although crude, the combination of the Mack-Wolfe and KS tests were able to identify proteins as well as a more sophisticated technique.

In classification problems, techniques such as Naïve Bayes and Logistic Regression are fit to training data which is assumed to be stable. However, the assumption of a longitudinal model is that those parameters will change with time. In this case proteins whose mean RFU in the Progressors becomes more different from the Controls as diagnosis draws nearer were selected.

A modeling approach was taken to construct a classifier based on the control data, where samples are scored based on similarity to the mean, or centroid, of the control samples. The model is essentially an outlier detector based on a signed Mahalanobis distance (MD), which is a multidimensional measure of the distance between a point P (Progressor sample) and a distribution D (Control data).

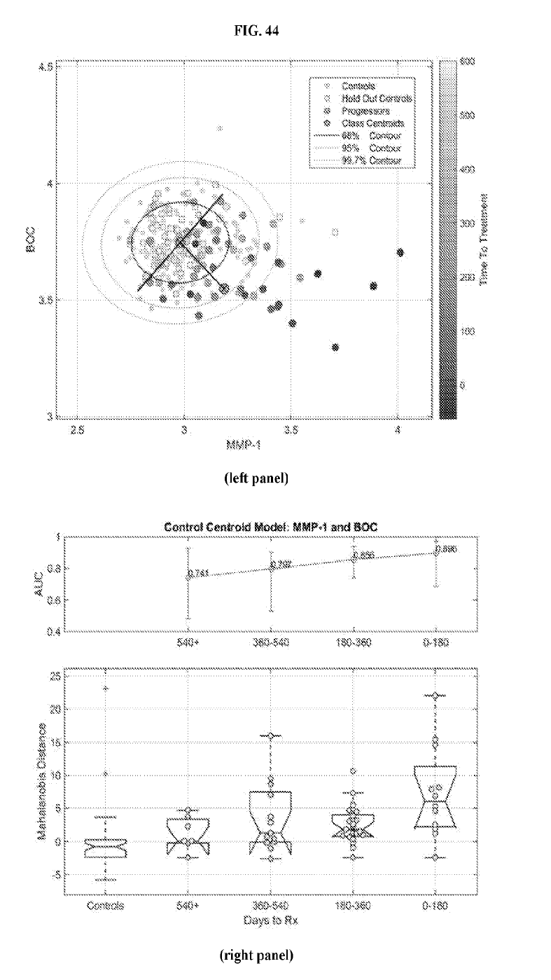

The left panel of FIG. 44 shows a representation of the model for the 2-dimensional case using two randomly chosen proteins from Table 11. The distribution of the control centroid is characterized by the matched controls, which is shown with contours of constant probability. For example, if a new control sample would only have a 5% chance of lying outside of the 95% contour, assuming it was taken from the same distribution as the Controls used to build the control centroid. For the model, TB data is only used to calculate the location of the TB centroid, which is used to orient an axis which describes the sign of the distance to the TB centroid. To reduce the impact of outliers on parameter estimates, robust methods were used (minimum covariance determinant).

As Progressors approach treatment, their samples should look less like controls and the signed MD will increase. The right panel of FIG. 44 shows example performance statistics. The simplicity of the Signed Mahanobis Distance model requires features to be optimally selected. Instead of using a greedy feature selection exhaustive feature selection was used, where every combination of [k] features from the list of 23 are used to fit a model and the model(s) with the optimal performance are selected based on this criterion.

To avoid the combinatorial explosion that comes with brute force methods, the number of proteins required for optimally performing models was investigated. All possible 2-6 feature models were fit using all TB samples with matched Controls, and the performance was evaluated using a set of non-matched controls (n=57) and the TB samples. No cross-validation was used. The cost function was the weighted AUC measure

$${AUC}_{Weighted} = \frac{2 ( {AUC}_{0 - 180} ) + ( {AUC}_{180 - 360} )}{3} ∈ [ 0 , 1 ]$$

Ten runs of three-fold cross-validation was used to estimate the performance of all models in the exhaustive model search. For each model size, a t-test was used to determine which models were not significantly different from the best model within cross-validated error at a 5% level. The left panel of FIG. 45 shows the weighted AUC statistic for all cross-validated performance estimates for all models. The right panel shows the combined performance estimates for each value of k. The median performance levels off at 3 proteins with the top models having the same performance at 4 proteins. Table A below shows the performance of all 4-protein models. Table B shows the performance of all 5-protein models. Table C shows 5 protein models with an AUC for 0 to 6 months of greater than 0.8.

To select a model, the longitudinal structure of the data was utilized by fitting a linear mixed effects model on the signed MD generated by each 5 feature model for Progressors with 2+ serial samples. Linear mixed effects models are extensions of linear regression, except certain coefficients can be allowed to randomly vary according to a grouping variable. In this instance, we allowed each Progressor to have a subject-specific intercept with the slope being the conventional linear regression slope calculated from all the data. This emphasized the slope of each Progressor's trajectory of signed MD while allowing for different starting points for each subject.

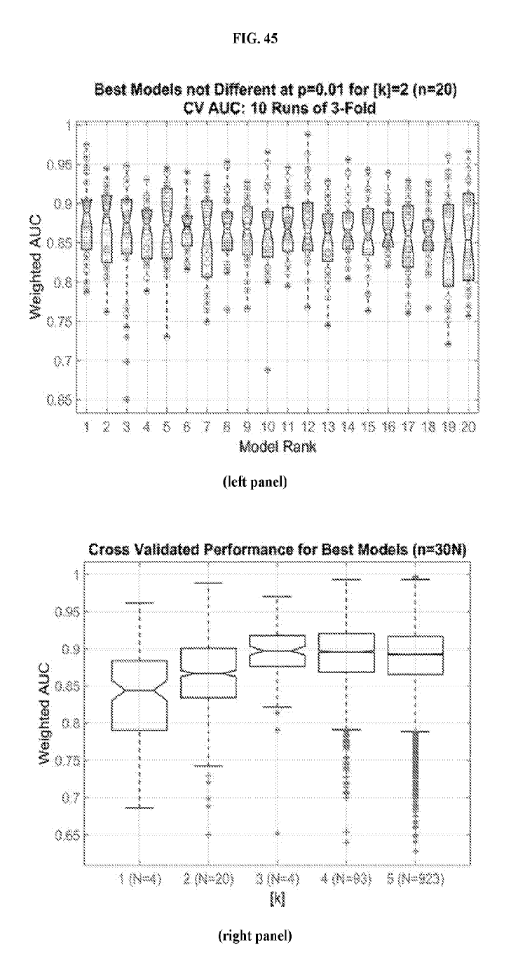

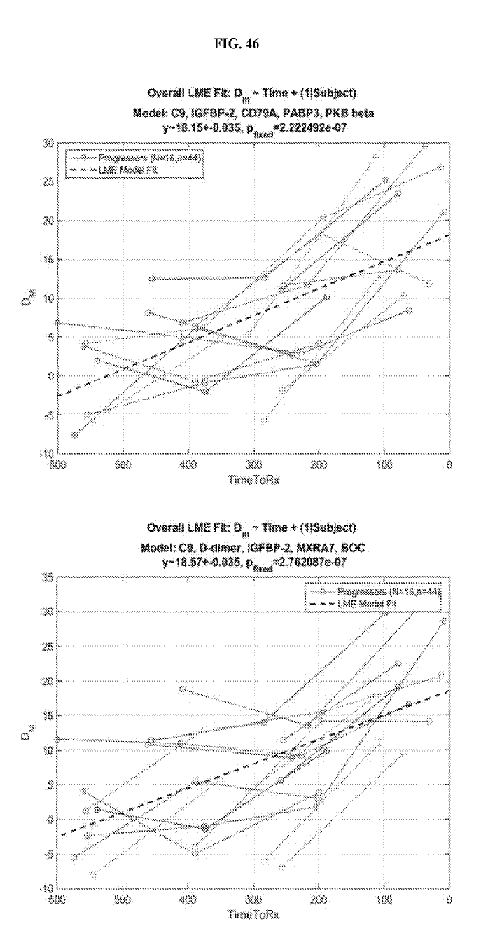

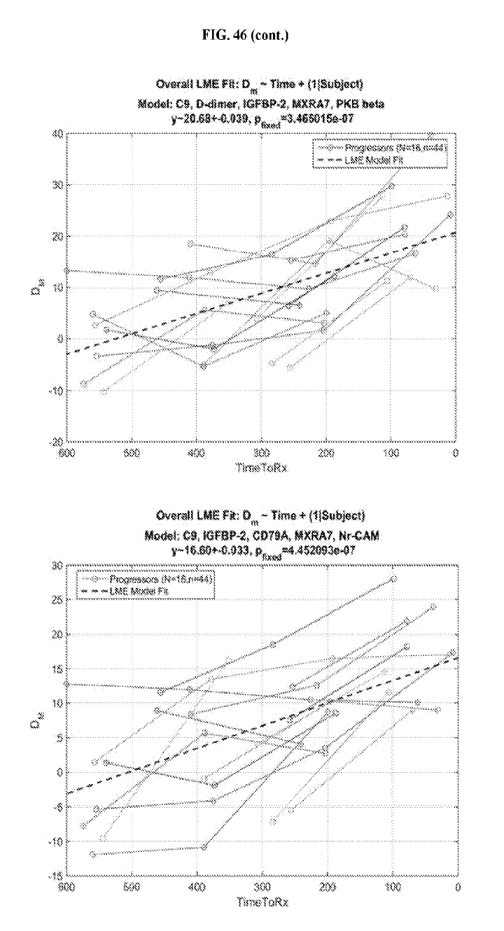

FIG. 46 shows the longitudinal trajectories of signed MD for Progressors as a function of Time to Treatment for the top 4 ranked models. The dashed black line shows the fitted slope to the data from the linear mixed effects model. Table 12 shows linear mixed effects model statistics for the top 25 ranked models.

TABLE 12
Table of statistics from a linear mixed model applied to the signed MD of the serials samples from Progressors.

| Rank | Protein 1 | Protein 2 | Protein 3 | Protein 4 | Protein 5 | p_{fixed} | Slope | Intercept |
|---|---|---|---|---|---|---|---|---|
| 1 | C9 | IGFBP-2 | CD79A | PABP3 | PKB beta | 2.22E−07 | −0.035 | 18.15 |
| 2 | C9 | D-dimer | IGFBP-2 | MXRA7 | BOC | 2.76E−07 | −0.035 | 18.57 |
| 3 | C9 | D-dimer | IGFBP-2 | MXRA7 | PKB beta | 3.47E−07 | −0.039 | 20.68 |
| 4 | C9 | IGFBP-2 | CD79A | MXRA7 | Nr-CAM | 4.45E−07 | −0.033 | 16.60 |
| 5 | C9 | IGFBP-2 | PABP3 | MXRA7 | Nr-CAM | 4.91E−07 | −0.037 | 18.76 |
| 6 | C9 | D-dimer | IGFBP-2 | CD79A | PKB beta | 7.15E−07 | −0.033 | 17.00 |
| 7 | C9 | IGFBP-2 | CA2D3 | PKB a/b/g | MXRA7 | 8.69E−07 | −0.036 | 19.35 |
| 8 | C9 | IGFBP-2 | CD79A | PABP3 | BOC | 9.69E−07 | −0.028 | 15.43 |
| 9 | C9 | IGFBP-2 | CD79A | NDUB4 | PKB beta | 1.14E−06 | −0.036 | 18.44 |
| 10 | C9 | IGFBP-2 | CA2D3 | PABP3 | MXRA7 | 1.28E−06 | −0.034 | 17.90 |
| 11 | C9 | PKB a/b/g | MXRA7 | CNTFR alpha | CD36 ANTIGEN | 1.33E−06 | −0.041 | 22.88 |
| 12 | C9 | IGFBP-2 | CD79A | PABP3 | MXRA7 | 1.42E−06 | −0.034 | 18.27 |
| 13 | C9 | IGFBP-2 | PKB a/b/g | PABP3 | PKB beta | 1.57E−06 | −0.038 | 20.33 |
| 14 | C9 | PABP3 | CNTFR alpha | CD36 ANTIGEN | PKB beta | 1.79E−06 | −0.039 | 21.70 |
| 15 | C9 | D-dimer | IGFBP-2 | PABP3 | PKB beta | 1.91E−06 | −0.034 | 18.42 |
| 16 | C9 | D-dimer | CD79A | PABP3 | MXRA7 | 1.93E−06 | −0.030 | 16.57 |
| 17 | C9 | IGFBP-2 | MXRA7 | NDUB4 | BOC | 1.97E−06 | −0.033 | 17.46 |
| 18 | C9 | PKB a/b/g | CNTFR alpha | CD36 ANTIGEN | PKB beta | 2.09E−06 | −0.043 | 24.02 |
| 19 | C9 | CA2D3 | PABP3 | CD36 ANTIGEN | PKB beta | 2.2E−06 | −0.041 | 20.66 |
| 20 | C9 | D-dimer | IGFBP-2 | PKB a/b/g | PKB beta | 2.27E−06 | −0.035 | 19.17 |
| 21 | C9 | IGFBP-2 | PABP3 | NDUB4 | PKB beta | 2.37E−06 | −0.033 | 17.73 |
| 22 | C9 | D-dimer | IGFBP-2 | CD79A | BOC | 2.61E−06 | −0.029 | 15.96 |
| 23 | C9 | D-dimer | IGFBP-2 | CD36 ANTIGEN | PKB beta | 2.61E−06 | −0.040 | 20.66 |
| 24 | C9 | D-dimer | PABP3 | NDUB4 | PKB beta | 2.65E−06 | −0.027 | 15.87 |
| 25 | C9 | D-dimer | IGFBP-2 | CNTFR alpha | BOC | 2.76E−06 | −0.037 | 19.40 |

To select a top model, D-dimer was excluded due to its involvement in the clotting cascade and subsequent absence from serum. Two proteins in the top ranked model (PABP3 and PKB beta) may be non-specific. In contrast, the fourth ranked model contains the same proteins except PABP3 and PKB beta are replaced by a matrix remodeling protein MXRA7 and immunoglobin-related protein Nr-CAM. Therefore, due to its performance and biological specificity, the fourth ranked model was chosen.

FIG. 47 shows the performance of this model, termed TRM5 for ‘Tuberculosis Responsive Model’, for each time bin. The left plot shows bootstrapped AUC estimates for Progressor samples within a time bin versus matched control samples, as well as a boxplot of the signed MD for each time bin. The right plot shows ROCs for each time bin versus matched controls. Table 13 shows the TRM model proteins.

TABLE 13
TRM5 Model proteins

| | | | | |
|---|---|---|---|---|
| C9 | IGFBP-2 | CD79A | MXRA7 | Nr-CAM |

The TRM5 model has an AUC of 0.72 for 300-540 days prior to treatment, an AUC of 0.8 for 180-300 days prior to treatment, and an AUC of 0.96 for 0-180 days prior to treatment. Thus, the TRM5 model performs reasonably well at predicting transition from latent to active TB 6-12 months prior to treatment, and very well at predicting transition from latent to active TB within 6 months of treatment. Tables A-C show additional panels that perform well for predicting the transition from latent to active TB.

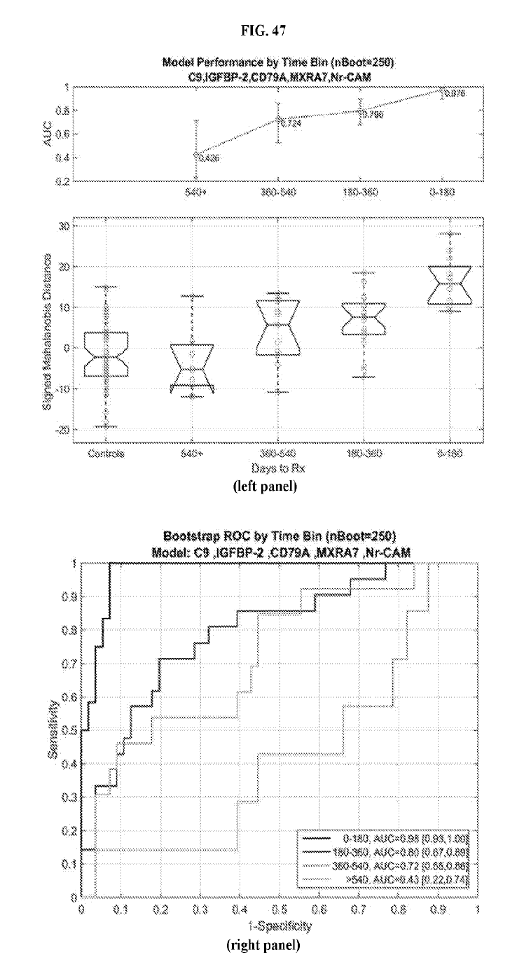

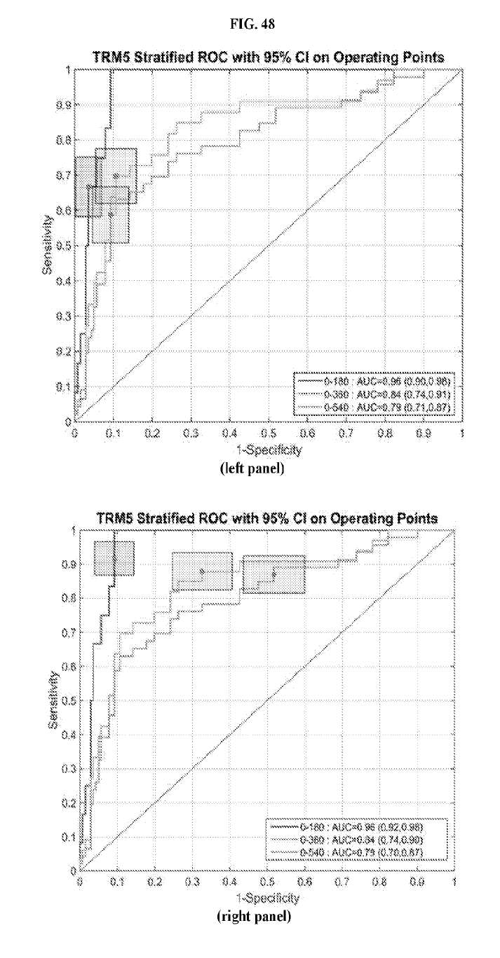

FIG. 48 shows ROCs with operating points stratified by time bin. The left plot shows operating points that emphasize specificity and the right plot emphasizes sensitivity. Shaded boxes show theoretical 95% joint confidence intervals for each operating point. Table 14 shows performance metrics for each emphasis stratified by time bin.

TABLE 14
Table of performance metrics for the TRM5 model
using different decision boundary thresholds.

| | Time | | | | | | | | | | |
|---|---|---|---|---|---|---|---|---|---|---|---|
| Emphasis | Bin | AUC | 95% CI | | Sensitivity | 95% CI | | Specificity | 95% CI | | Dm |
| Specificity | 0-180 | 0.96 | 0.92 | 0.98 | 0.67 | 0.58 | 0.75 | 0.96 | 1.00 | 0.93 | 14.55 |
| | 0-360 | 0.84 | 0.74 | 0.90 | 0.70 | 0.62 | 0.77 | 0.89 | 0.95 | 0.84 | 7.59 |
| | 0-540 | 0.79 | 0.70 | 0.87 | 0.59 | 0.51 | 0.67 | 0.91 | 0.95 | 0.86 | 8.41 |
| Sensitivity | 0-180 | 0.96 | 0.92 | 0.98 | 0.92 | 0.87 | 0.97 | 0.91 | 0.04 | 0.14 | 8.36 |
| | 0-360 | 0.84 | 0.74 | 0.90 | 0.88 | 0.82 | 0.93 | 0.77 | 0.25 | 0.41 | 2.69 |
| | 0-540 | 0.79 | 0.70 | 0.87 | 0.87 | 0.81 | 0.92 | 0.48 | 0.44 | 0.60 | −1.91 |

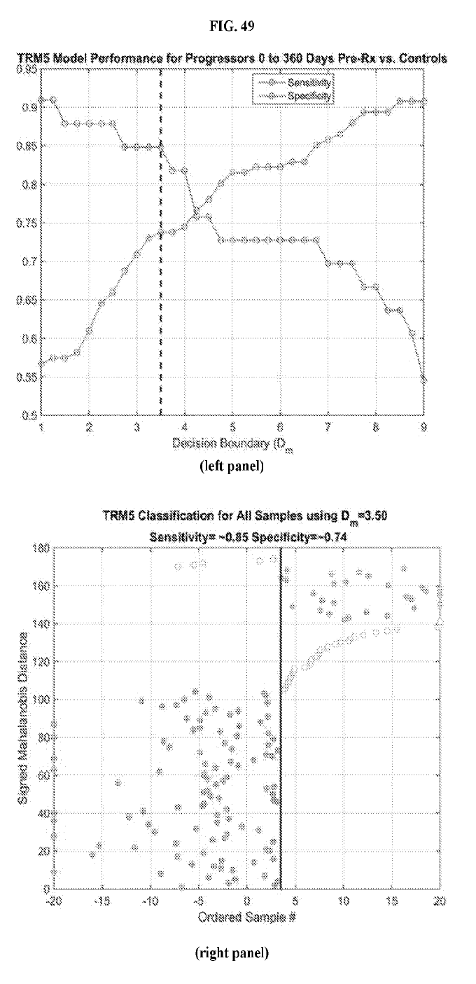

To select an operating point, a signed MD decisions boundary on [1, 9] was used to calculate the sensitivity and specificity. The left panel of FIG. 49 shows each as a function of the decision boundary for Progressors 0-360 before treatment versus controls. Where to operate on this spectrum depends on the intended use of the test. Given that this is a diagnostic product where positives will be either treated or followed for a period of time, a high sensitivity would be important.

An operating point of 3.5 was chosen as a balance between high sensitivity (0.85) and moderate specificity (0.74). The left plot of FIG. 49 shows a decision boundary plot using a signed MD of 3.5.

### Example 4

### Model Verification

A verification set was created comprising a) 38 samples from 15 progressors, with 12 samples collected after treatment; and b) 93 control samples from 36 participants, to confirm the performance of the TRM5 model.

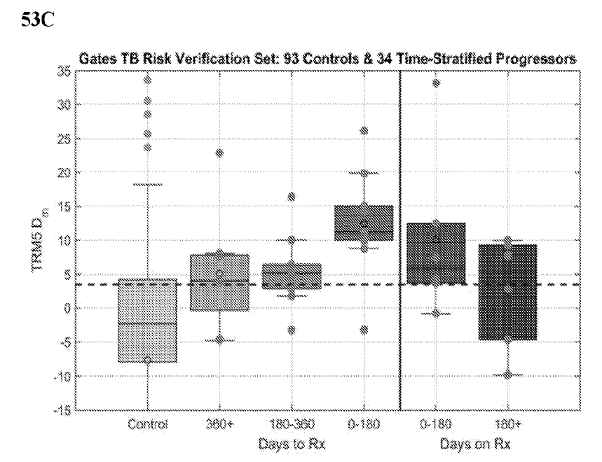

FIG. 53A shows the ROC curve for the all prospective samples. FIG. 53B shows time stratified ROC curves. Across all samples and time-points, the TRM5 model performed with AUC=0.76 (0.66, 0.84) in the verification set, which is very similar to the AUC=0.79 (0.70, 0.87) obtained in the Training set. Also consistent between training and verification was that the performance was best in the 0-180 days Pre-Rx. This data, using a blinded verification set, confirms the performance of the TRM5 model.

TRM5 was designed to be a responsive model, where the response metric (Mahalanobis Distance, or Dm) increases monotonically to the day of diagnosis. The left side of FIG. 53C shows a boxplot of Dm stratified by the time window. In the verification set, the TRM5 model produced TB risk scores below the threshold for progression in the controls. In the progressors, the TB risk scores increased toward the day of diagnosis and then dropped after treatment was initiated. Thus, the TB risk scores calculated with the TRM5 model were consistent between training and verification samples with regard to distinguishing cases from controls and reflecting the longitudinal changes.

The foregoing embodiments and examples are intended only as examples. No particular embodiment, example, or element of a particular embodiment or example is to be construed as a critical, required, or essential element or feature of any of the claims. Various alterations, modifications, substitutions, and other variations can be made to the disclosed embodiments without departing from the scope of the present application, which is defined by the appended claims. The specification, including the figures and examples, is to be regarded in an illustrative manner, rather than a restrictive one, and all such modifications and substitutions are intended to be included within the scope of the application. Steps recited in any of the method or process claims may be executed in any feasible order and are not limited to an order presented in any of the embodiments, the examples, or the claims. Further, in any of the aforementioned methods, one or more specifically listed biomarkers can be specifically excluded either as an individual biomarker or as a biomarker from any panel.

### REFERENCES

The following references are herein incorporated by reference in their entireties.
Gold L. et al. Aptamer-based multiplexed proteomic technology for biomarker discovery. PLoS One 5, e15004 (2010).
Gold L., Walker J J, Wilcox S K, Williams S. Advances in human proteomics at high scale with the SOMAscan proteomics platform. N Biotechnol 29, 543-9 (2012).
De Groote M A, Nahid P, Jarlsberg L, Johnson J L, Weiner M, Muzanyi G, Janjic N, Sterling D G, Ochsner U A. Elucidating novel serum biomarkers associated with pulmonary tuberculosis treatment. PLoS One 8, e61002 (2013).
Hanekom W A, Hawkridge A, Mahomed H, Scriba T J, Tameris M, Hughes J, Hatherill M, Day C L, Hussey G D. SATVI—after 10 years closing in on a new and better vaccine to prevent tuberculosis. S Afr Med J. 102:438-41 (2012).
Maitournam A, Simon R. On the efficiency of targeted clinical trials. Stat Med. 24:329-39 (2005).

TABLE A
4 Protein Models

| Prot1 | Prot2 | Prot3 | Prot4 | AUC_0_180 | AUC_180_360 | AUC_Weight | JTpval |
|---|---|---|---|---|---|---|---|
| D-dimer | CD79A | MXRA7 | NDUB4 | 0.969 | 0.802 | 0.915 | 0.021 |
| D-dimer | IP-10 | CA2D3 | MXRA7 | 0.963 | 0.782 | 0.912 | 0.027 |
| C9 | PABP3 | MXRA7 | NDUB4 | 0.984 | 0.759 | 0.911 | 0.005 |
| C9 | IGFBP-2 | CA2D3 | MXRA7 | 0.975 | 0.755 | 0.909 | 0.015 |
| C9 | D-dimer | IP-10 | PABP3 | 0.968 | 0.779 | 0.906 | 0.017 |
| C9 | MXRA7 | NDUB4 | JKIP3 | 0.980 | 0.753 | 0.906 | 0.003 |
| C9 | D-dimer | PABP3 | MXRA7 | 0.980 | 0.753 | 0.906 | 0.020 |
| D-dimer | CD79A | PABP3 | PCI | 0.955 | 0.802 | 0.906 | 0.050 |
| C9 | D-dimer | MXRA7 | NDUB4 | 0.979 | 0.751 | 0.906 | 0.011 |
| D-dimer | IP-10 | IGFBP-2 | MXRA7 | 0.967 | 0.757 | 0.903 | 0.009 |
| D-dimer | IP-10 | IGFBP-2 | PABP3 | 0.973 | 0.752 | 0.902 | 0.011 |
| C9 | D-dimer | IP-10 | MXRA7 | 0.970 | 0.761 | 0.902 | 0.010 |
| D-dimer | IP-10 | MXRA7 | JKIP3 | 0.956 | 0.803 | 0.902 | 0.015 |
| D-dimer | IGFBP-2 | CA2D3 | MXRA7 | 0.978 | 0.767 | 0.901 | 0.022 |
| C9 | IP-10 | MXRA7 | NDUB4 | 0.971 | 0.767 | 0.901 | 0.007 |
| C9 | D-dimer | MXRA7 | Nr-CAM | 0.972 | 0.768 | 0.901 | 0.023 |
| C9 | IP-10 | MXRA7 | JKIP3 | 0.965 | 0.764 | 0.900 | 0.010 |
| IP-10 | CA2D3 | MXRA7 | NDUB4 | 0.968 | 0.761 | 0.900 | 0.016 |
| C9 | IP-10 | PABP3 | NDUB4 | 0.960 | 0.770 | 0.899 | 0.010 |
| C9 | IP-10 | CA2D3 | MXRA7 | 0.967 | 0.768 | 0.899 | 0.005 |
| C9 | PABP3 | MXRA7 | JKIP3 | 0.972 | 0.777 | 0.898 | 0.004 |
| C9 | NDUB4 | PKB beta | JKIP3 | 0.969 | 0.755 | 0.898 | 0.004 |
| C9 | MXRA7 | NDUB4 | PKB beta | 0.980 | 0.751 | 0.898 | 0.007 |
| IP-10 | PKB a/b/g | PABP3 | NDUB4 | 0.948 | 0.809 | 0.898 | 0.032 |
| D-dimer | IP-10 | PKB a/b/g | MXRA7 | 0.959 | 0.780 | 0.898 | 0.037 |
| D-dimer | CA2D3 | MXRA7 | NDUB4 | 0.964 | 0.758 | 0.897 | 0.010 |
| C9 | PABP3 | NDUB4 | PCI | 0.966 | 0.752 | 0.896 | 0.028 |
| D-dimer | IP-10 | MXRA7 | NDUB4 | 0.964 | 0.757 | 0.895 | 0.018 |
| D-dimer | IP-10 | PABP3 | NDUB4 | 0.967 | 0.766 | 0.894 | 0.010 |
| D-dimer | IP-10 | CA2D3 | CD36 | 0.966 | 0.770 | 0.894 | 0.010 |
| | | | ANTIGEN | | | | |
| D-dimer | IP-10 | CD79A | MXRA7 | 0.964 | 0.775 | 0.894 | 0.018 |
| C9 | IP-10 | PKB a/b/g | MXRA7 | 0.967 | 0.752 | 0.893 | 0.016 |
| C9 | MMP-2 | PABP3 | MXRA7 | 0.968 | 0.755 | 0.893 | 0.027 |
| C9 | CD79A | PABP3 | JKIP3 | 0.950 | 0.758 | 0.891 | 0.003 |
| C9 | MMP-2 | CNTFR | NDUB4 | 0.966 | 0.769 | 0.890 | 0.027 |
| | | alpha | | | | | |
| D-dimer | MMP-2 | CD79A | PABP3 | 0.930 | 0.789 | 0.889 | 0.036 |
| D-dimer | MMP-2 | PABP3 | PCI | 0.938 | 0.804 | 0.888 | 0.042 |
| IP-10 | CD79A | PKB a/b/g | MXRA7 | 0.959 | 0.775 | 0.888 | 0.024 |
| D-dimer | CA2D3 | PABP3 | PCI | 0.937 | 0.791 | 0.882 | 0.027 |
| IP-10 | PABP3 | MXRA7 | NDUB4 | 0.954 | 0.763 | 0.881 | 0.015 |

TABLE B
5 Protein Models

| Prot1 | Prot2 | Prot3 | Prot4 | Prot5 | AUC_0_180 | AUC_180_360 | AUC_Weight | JTpval |
|---|---|---|---|---|---|---|---|---|
| C9 | D-dimer | IGFBP-2 | MMP-2 | MXRA7 | 0.982 | 0.774 | 0.917 | 0.018 |
| C9 | D-dimer | PABP3 | MXRA7 | NDUB4 | 0.979 | 0.792 | 0.917 | 0.014 |
| C9 | D-dimer | IGFBP-2 | CA2D3 | MXRA7 | 0.977 | 0.791 | 0.917 | 0.004 |
| MMP-1 | IP-10 | IGFBP-2 | PABP3 | MXRA7 | 0.960 | 0.812 | 0.917 | 0.022 |
| C9 | D-dimer | MMP-2 | MXRA7 | NDUB4 | 0.982 | 0.793 | 0.916 | 0.050 |
| C9 | D-dimer | MMP-2 | PABP3 | MXRA7 | 0.971 | 0.803 | 0.913 | 0.034 |
| D-dimer | IGFBP-2 | CA2D3 | PABP3 | MXRA7 | 0.964 | 0.775 | 0.913 | 0.025 |
| MMP-1 | IP-10 | IGFBP-2 | MXRA7 | NDUB4 | 0.960 | 0.821 | 0.913 | 0.023 |
| C9 | D-dimer | MMP-2 | MXRA7 | JKIP3 | 0.971 | 0.797 | 0.913 | 0.032 |
| MMP-1 | C9 | IP-10 | MXRA7 | BOC | 0.967 | 0.789 | 0.911 | 0.037 |
| C9 | D-dimer | MXRA7 | NDUB4 | JKIP3 | 0.981 | 0.788 | 0.911 | 0.004 |
| MMP-1 | C9 | IP-10 | MXRA7 | CNTFR | 0.957 | 0.786 | 0.910 | 0.024 |
| | | | | alpha | | | | |
| D-dimer | IP-10 | IGFBP-2 | CA2D3 | PABP3 | 0.975 | 0.796 | 0.910 | 0.010 |
| C9 | IGFBP-2 | PABP3 | MXRA7 | Nr-CAM | 0.973 | 0.777 | 0.910 | 0.007 |
| C9 | IP-10 | MXRA7 | NDUB4 | JKIP3 | 0.963 | 0.777 | 0.910 | 0.010 |
| C9 | D-dimer | MMP-2 | PABP3 | NDUB4 | 0.982 | 0.764 | 0.909 | 0.031 |
| MMP-1 | IP-10 | PABP3 | MXRA7 | NDUB4 | 0.964 | 0.824 | 0.909 | 0.023 |
| C9 | D-dimer | PABP3 | MXRA7 | Nr-CAM | 0.958 | 0.788 | 0.909 | 0.037 |
| MMP-1 | IP-10 | IGFBP-2 | CA2D3 | MXRA7 | 0.959 | 0.823 | 0.909 | 0.027 |
| C9 | MMP-2 | PABP3 | MXRA7 | NDUB4 | 0.985 | 0.782 | 0.908 | 0.022 |
| C9 | D-dimer | IP-10 | CA2D3 | MXRA7 | 0.973 | 0.798 | 0.908 | 0.006 |
| MMP-1 | IP-10 | PKB | PABP3 | MXRA7 | 0.945 | 0.823 | 0.907 | 0.046 |
| | | a/b/g | | | | | | |
| MMP-1 | IP-10 | CA2D3 | PABP3 | MXRA7 | 0.955 | 0.822 | 0.907 | 0.032 |
| D-dimer | IGFBP-2 | CA2D3 | CD79A | MXRA7 | 0.976 | 0.773 | 0.907 | 0.013 |
| MMP-1 | IP-10 | IGFBP-2 | PABP3 | CD36 | 0.967 | 0.801 | 0.907 | 0.029 |
| | | | | ANTIGEN | | | | |
| D-dimer | IP-10 | IGFBP-2 | PABP3 | CNTFR | 0.973 | 0.767 | 0.907 | 0.003 |
| | | | | alpha | | | | |
| MMP-1 | IP-10 | IGFBP-2 | MXRA7 | CNTFR | 0.959 | 0.819 | 0.906 | 0.017 |
| | | | | alpha | | | | |
| C9 | D-dimer | IP-10 | PABP3 | Nr-CAM | 0.967 | 0.794 | 0.906 | 0.018 |
| C9 | IP-10 | CA2D3 | PKB a/b/g | MXRA7 | 0.960 | 0.812 | 0.906 | 0.009 |
| D-dimer | IP-10 | IGFBP-2 | PABP3 | CD36 | 0.977 | 0.768 | 0.906 | 0.011 |
| | | | | ANTIGEN | | | | |
| C9 | IP-10 | PKB | MXRA7 | NDUB4 | 0.971 | 0.753 | 0.906 | 0.012 |
| | | a/b/g | | | | | | |
| D-dimer | IP-10 | IGFBP-2 | PABP3 | JKIP3 | 0.967 | 0.777 | 0.906 | 0.010 |
| C9 | D-dimer | IP-10 | PKB a/b/g | MXRA7 | 0.963 | 0.770 | 0.905 | 0.019 |
| D-dimer | IP-10 | IGFBP-2 | CA2D3 | JKIP3 | 0.967 | 0.787 | 0.905 | 0.005 |
| MMP-1 | IP-10 | IGFBP-2 | CD36 | PKB beta | 0.958 | 0.796 | 0.905 | 0.037 |
| | | | ANTIGEN | | | | | |
| MMP-1 | IP-10 | IGFBP-2 | PGCB | MXRA7 | 0.958 | 0.770 | 0.905 | 0.022 |
| D-dimer | IP-10 | IGFBP-2 | PABP3 | NDUB4 | 0.975 | 0.791 | 0.905 | 0.008 |
| C9 | PABP3 | NDUB4 | PKB beta | JKIP3 | 0.977 | 0.777 | 0.905 | 0.006 |
| C9 | IGFBP-2 | CD79A | PABP3 | MXRA7 | 0.987 | 0.757 | 0.905 | 0.003 |
| C9 | D-dimer | NPS | MMP-2 | MXRA7 | 0.971 | 0.782 | 0.905 | 0.050 |
| C9 | D-dimer | IGFBP-2 | MXRA7 | Nr-CAM | 0.972 | 0.772 | 0.905 | 0.008 |
| C9 | D-dimer | IP-10 | CA2D3 | PKB | 0.970 | 0.771 | 0.905 | 0.019 |
| | | | | a/b/g | | | | |
| C9 | CA2D3 | CD36 | PKB beta | JKIP3 | 0.974 | 0.765 | 0.904 | 0.021 |
| | | ANTIGEN | | | | | | |
| C9 | D-dimer | CA2D3 | PABP3 | JKIP3 | 0.969 | 0.761 | 0.904 | 0.012 |
| C9 | D-dimer | IP-10 | PABP3 | JKIP3 | 0.964 | 0.759 | 0.904 | 0.015 |
| C9 | D-dimer | MMP-2 | PKB a/b/g | MXRA7 | 0.972 | 0.780 | 0.904 | 0.022 |
| MMP-1 | IP-10 | PABP3 | MXRA7 | PCI | 0.950 | 0.817 | 0.904 | 0.042 |
| C9 | D-dimer | IGFBP-2 | PABP3 | MXRA7 | 0.971 | 0.780 | 0.904 | 0.007 |
| MMP-1 | IP-10 | PABP3 | MXRA7 | CNTFR | 0.961 | 0.819 | 0.904 | 0.038 |
| | | | | alpha | | | | |
| C9 | D-dimer | CA2D3 | MXRA7 | JKIP3 | 0.979 | 0.779 | 0.904 | 0.007 |
| C9 | IP-10 | PABP3 | MXRA7 | NDUB4 | 0.974 | 0.757 | 0.904 | 0.007 |
| MMP-1 | IP-10 | IGFBP-2 | PKB a/b/g | MXRA7 | 0.956 | 0.804 | 0.904 | 0.037 |
| MMP-1 | IP-10 | IGFBP-2 | PABP3 | PCI | 0.947 | 0.805 | 0.903 | 0.040 |
| C9 | CA2D3 | MXRA7 | NDUB4 | JKIP3 | 0.981 | 0.768 | 0.903 | 0.012 |
| IP-10 | CA2D3 | PABP3 | CNTFR | NDUB4 | 0.962 | 0.771 | 0.903 | 0.010 |
| | | | alpha | | | | | |
| C9 | D-dimer | PABP3 | MXRA7 | JKIP3 | 0.967 | 0.786 | 0.903 | 0.013 |
| C9 | D-dimer | IP-10 | MXRA7 | JKIP3 | 0.963 | 0.774 | 0.903 | 0.005 |
| C9 | D-dimer | CA2D3 | PKB a/b/g | MXRA7 | 0.976 | 0.759 | 0.903 | 0.016 |
| MMP-1 | IP-10 | IGFBP-2 | MXRA7 | PKB beta | 0.960 | 0.778 | 0.903 | 0.029 |
| MMP-1 | IP-10 | IGFBP-2 | MXRA7 | PCI | 0.953 | 0.813 | 0.903 | 0.030 |
| MMP-1 | IP-10 | CA2D3 | MXRA7 | NDUB4 | 0.956 | 0.811 | 0.903 | 0.050 |
| C9 | D-dimer | IGFBP-2 | CNTFR | BOC | 0.973 | 0.764 | 0.903 | 0.002 |
| | | | alpha | | | | | |
| D-dimer | IP-10 | MXRA7 | CD36 | JKIP3 | 0.973 | 0.770 | 0.903 | 0.016 |
| | | | ANTIGEN | | | | | |
| C9 | D-dimer | IP-10 | MMP-2 | NDUB4 | 0.954 | 0.796 | 0.903 | 0.026 |
| C9 | PABP3 | MXRA7 | NDUB4 | JKIP3 | 0.978 | 0.772 | 0.903 | 0.005 |
| MMP-1 | IP-10 | IGFBP-2 | PABP3 | NDUB4 | 0.959 | 0.802 | 0.903 | 0.044 |
| C9 | IGFBP-2 | CA2D3 | PABP3 | MXRA7 | 0.974 | 0.755 | 0.903 | 0.002 |
| D-dimer | IP-10 | CA2D3 | MXRA7 | CD36 | 0.968 | 0.755 | 0.903 | 0.016 |
| | | | | ANTIGEN | | | | |
| D-dimer | IP-10 | IGFBP-2 | CD79A | NDUB4 | 0.969 | 0.774 | 0.902 | 0.006 |
| C9 | D-dimer | PKB | MXRA7 | NDUB4 | 0.975 | 0.764 | 0.902 | 0.022 |
| | | a/b/g | | | | | | |
| C9 | D-dimer | MXRA7 | CNTFR | CD36 | 0.973 | 0.772 | 0.902 | 0.004 |
| | | | alpha | ANTIGEN | | | | |
| MMP-1 | IP-10 | PGCB | MXRA7 | CNTFR | 0.954 | 0.815 | 0.902 | 0.021 |
| | | | | alpha | | | | |
| IP-10 | IGFBP-2 | CD79A | PABP3 | CNTFR | 0.965 | 0.771 | 0.902 | 0.015 |
| | | | | alpha | | | | |
| MMP-1 | PKB a/b/g | NDUB4 | BOC | PKB beta | 0.958 | 0.763 | 0.902 | 0.020 |
| C9 | PABP3 | MXRA7 | CNTFR | CD36 | 0.969 | 0.756 | 0.902 | 0.009 |
| | | | alpha | ANTIGEN | | | | |
| C9 | D-dimer | IGFBP-2 | MXRA7 | NDUB4 | 0.982 | 0.769 | 0.902 | 0.014 |
| D-dimer | IP-10 | IGFBP-2 | MXRA7 | NDUB4 | 0.973 | 0.772 | 0.902 | 0.011 |
| C9 | D-dimer | IP-10 | MXRA7 | NDUB4 | 0.974 | 0.761 | 0.902 | 0.009 |
| C9 | D-dimer | IGFBP-2 | CD79A | CNTFR | 0.967 | 0.752 | 0.902 | 0.006 |
| | | | | alpha | | | | |
| D-dimer | IP-10 | CA2D3 | PABP3 | CD36 | 0.968 | 0.766 | 0.902 | 0.012 |
| | | | | ANTIGEN | | | | |
| C9 | D-dimer | PABP3 | NDUB4 | BOC | 0.970 | 0.774 | 0.902 | 0.009 |
| C9 | D-dimer | CA2D3 | MXRA7 | PKB beta | 0.975 | 0.757 | 0.902 | 0.009 |
| C9 | IGFBP-2 | CA2D3 | CD79A | MXRA7 | 0.977 | 0.750 | 0.902 | 0.006 |
| C9 | D-dimer | CA2D3 | MXRA7 | Nr-CAM | 0.970 | 0.759 | 0.902 | 0.027 |
| C9 | D-dimer | IP-10 | PABP3 | NDUB4 | 0.971 | 0.768 | 0.902 | 0.013 |
| MMP-1 | IP-10 | PKB | MXRA7 | PKB beta | 0.973 | 0.781 | 0.901 | 0.028 |
| | | a/b/g | | | | | | |
| IP-10 | IGFBP-2 | PABP3 | CNTFR | PCI | 0.951 | 0.781 | 0.901 | 0.017 |
| | | | alpha | | | | | |
| C9 | IP-10 | PABP3 | MXRA7 | CNTFR | 0.970 | 0.762 | 0.901 | 0.004 |
| | | | | alpha | | | | |
| C9 | D-dimer | IGFBP-2 | MXRA7 | BOC | 0.970 | 0.777 | 0.901 | 0.004 |
| C9 | IP-10 | CA2D3 | MXRA7 | NDUB4 | 0.973 | 0.759 | 0.901 | 0.014 |
| C9 | D-dimer | PKB | PABP3 | MXRA7 | 0.966 | 0.774 | 0.901 | 0.010 |
| | | a/b/g | | | | | | |
| C9 | IP-10 | MMP-2 | PABP3 | NDUB4 | 0.969 | 0.758 | 0.901 | 0.044 |
| D-dimer | IP-10 | PABP3 | CNTFR | CD36 | 0.971 | 0.759 | 0.901 | 0.008 |
| | | | alpha | ANTIGEN | | | | |
| IP-10 | IGFBP-2 | PABP3 | NDUB4 | PCI | 0.948 | 0.802 | 0.901 | 0.007 |
| C9 | IP-10 | IGFBP-2 | PABP3 | MXRA7 | 0.972 | 0.758 | 0.901 | 0.010 |
| C9 | D-dimer | IP-10 | CD36 | BOC | 0.960 | 0.782 | 0.901 | 0.015 |
| | | | ANTIGEN | | | | | |
| MMP-1 | IP-10 | IGFBP-2 | PABP3 | CNTFR | 0.952 | 0.784 | 0.901 | 0.016 |
| | | | | alpha | | | | |
| IP-10 | IGFBP-2 | CA2D3 | PABP3 | MXRA7 | 0.960 | 0.767 | 0.901 | 0.005 |
| C9 | D-dimer | IP-10 | CA2D3 | NDUB4 | 0.969 | 0.767 | 0.901 | 0.004 |
| IP-10 | PKB a/b/g | PABP3 | Nr-CAM | PKB beta | 0.956 | 0.778 | 0.900 | 0.018 |
| IP-10 | CD79A | PKB | PABP3 | PKB beta | 0.974 | 0.761 | 0.900 | 0.010 |
| | | a/b/g | | | | | | |
| MMP-1 | IP-10 | PABP3 | MXRA7 | JKIP3 | 0.953 | 0.821 | 0.900 | 0.046 |
| MMP-1 | IP-10 | CD79A | PABP3 | MXRA7 | 0.942 | 0.815 | 0.900 | 0.038 |
| MMP-1 | C9 | IP-10 | PKB a/b/g | MXRA7 | 0.957 | 0.764 | 0.900 | 0.043 |
| C9 | D-dimer | IP-10 | MXRA7 | Nr-CAM | 0.959 | 0.803 | 0.900 | 0.012 |
| C9 | D-dimer | IP-10 | IGFBP-2 | MXRA7 | 0.963 | 0.761 | 0.900 | 0.005 |
| D-dimer | IP-10 | CA2D3 | MXRA7 | JKIP3 | 0.959 | 0.767 | 0.900 | 0.015 |
| D-dimer | IGFBP-2 | CA2D3 | PABP3 | PCI | 0.958 | 0.781 | 0.900 | 0.015 |
| C9 | IP-10 | IGFBP-2 | PABP3 | Nr-CAM | 0.957 | 0.766 | 0.900 | 0.004 |
| D-dimer | IP-10 | MXRA7 | Nr-CAM | CD36 | 0.961 | 0.766 | 0.899 | 0.015 |
| | | | | ANTIGEN | | | | |
| C9 | D-dimer | IP-10 | CA2D3 | CD36 | 0.975 | 0.765 | 0.899 | 0.006 |
| | | | | ANTIGEN | | | | |
| MMP-1 | IP-10 | IGFBP-2 | MXRA7 | CD36 | 0.962 | 0.784 | 0.899 | 0.017 |
| | | | | ANTIGEN | | | | |
| MMP-1 | IGFBP-2 | MXRA7 | CNTFR | NDUB4 | 0.948 | 0.787 | 0.899 | 0.041 |
| | | | alpha | | | | | |
| C9 | D-dimer | IGFBP-2 | PKB a/b/g | MXRA7 | 0.967 | 0.758 | 0.899 | 0.008 |
| D-dimer | IP-10 | CD36 | NDUB4 | PCI | 0.967 | 0.758 | 0.899 | 0.016 |
| | | ANTIGEN | | | | | | |
| C9 | IP-10 | MMP-2 | MXRA7 | NDUB4 | 0.974 | 0.751 | 0.899 | 0.022 |
| MMP-1 | IP-10 | CD36 | NDUB4 | PKB beta | 0.950 | 0.787 | 0.899 | 0.036 |
| | | ANTIGEN | | | | | | |
| C9 | CA2D3 | PKB | MXRA7 | BOC | 0.978 | 0.759 | 0.899 | 0.008 |
| | | a/b/g | | | | | | |
| C9 | D-dimer | IGFBP-2 | Nr-CAM | PKB beta | 0.966 | 0.771 | 0.899 | 0.007 |
| MMP-1 | C9 | IP-10 | IGFBP-2 | BOC | 0.960 | 0.784 | 0.898 | 0.030 |
| MMP-1 | IP-10 | IGFBP-2 | CD79A | CNTFR | 0.946 | 0.795 | 0.898 | 0.027 |
| | | | | alpha | | | | |
| C9 | IP-10 | MXRA7 | PKB beta | JKIP3 | 0.967 | 0.771 | 0.898 | 0.008 |
| MMP-1 | IP-10 | CNTFR | CD36 | PKB beta | 0.947 | 0.776 | 0.898 | 0.037 |
| | | alpha | ANTIGEN | | | | | |
| C9 | D-dimer | IP-10 | CA2D3 | PABP3 | 0.962 | 0.772 | 0.898 | 0.012 |
| C9 | MMP-2 | MXRA7 | PKB beta | JKIP3 | 0.972 | 0.753 | 0.898 | 0.036 |
| C9 | MMP-2 | PKB | CNTFR | NDUB4 | 0.970 | 0.751 | 0.898 | 0.023 |
| | | a/b/g | alpha | | | | | |
| C9 | CA2D3 | PABP3 | MXRA7 | JKIP3 | 0.964 | 0.773 | 0.898 | 0.008 |
| MMP-1 | IP-10 | CA2D3 | CD36 | NDUB4 | 0.950 | 0.792 | 0.898 | 0.049 |
| | | | ANTIGEN | | | | | |
| IP-10 | CA2D3 | PABP3 | MXRA7 | Ephrin- | 0.952 | 0.767 | 0.898 | 0.016 |
| | | | | A3 | | | | |
| C9 | IP-10 | CA2D3 | PABP3 | MXRA7 | 0.968 | 0.798 | 0.898 | 0.016 |
| MMP-1 | IP-10 | PKB | CNTFR | PKB beta | 0.970 | 0.772 | 0.897 | 0.043 |
| | | a/b/g | alpha | | | | | |
| MMP-1 | IP-10 | MXRA7 | CD36 | PKB beta | 0.968 | 0.765 | 0.897 | 0.041 |
| | | | ANTIGEN | | | | | |
| MMP-1 | IP-10 | PABP3 | CNTFR | NDUB4 | 0.949 | 0.807 | 0.897 | 0.036 |
| | | | alpha | | | | | |
| C9 | IP-10 | PABP3 | MXRA7 | Nr-CAM | 0.966 | 0.767 | 0.897 | 0.007 |
| C9 | D-dimer | PKB | NDUB4 | BOC | 0.963 | 0.750 | 0.897 | 0.013 |
| | | a/b/g | | | | | | |
| C9 | IP-10 | PKB | MXRA7 | JKIP3 | 0.967 | 0.768 | 0.897 | 0.007 |
| | | a/b/g | | | | | | |
| C9 | D-dimer | NDUB4 | PKB beta | JKIP3 | 0.970 | 0.758 | 0.897 | 0.008 |
| C9 | D-dimer | CA2D3 | PABP3 | BOC | 0.966 | 0.757 | 0.897 | 0.006 |
| D-dimer | IP-10 | CA2D3 | MXRA7 | NDUB4 | 0.965 | 0.762 | 0.897 | 0.011 |
| C9 | D-dimer | IP-10 | MXRA7 | BOC | 0.968 | 0.754 | 0.897 | 0.020 |
| C9 | IP-10 | CD79A | PABP3 | MXRA7 | 0.965 | 0.761 | 0.897 | 0.011 |
| C9 | IP-10 | IGFBP-2 | MXRA7 | JKIP3 | 0.965 | 0.765 | 0.897 | 0.006 |
| C9 | IP-10 | MXRA7 | NDUB4 | PKB beta | 0.971 | 0.764 | 0.897 | 0.018 |
| C9 | D-dimer | IGFBP-2 | NDUB4 | PKB beta | 0.970 | 0.763 | 0.897 | 0.003 |
| MMP-1 | IP-10 | MXRA7 | CNTFR | NDUB4 | 0.950 | 0.822 | 0.897 | 0.039 |
| | | | alpha | | | | | |
| MMP-1 | IP-10 | IGFBP-2 | MXRA7 | JKIP3 | 0.956 | 0.765 | 0.897 | 0.021 |
| C9 | IP-10 | IGFBP-2 | MXRA7 | CNTFR | 0.964 | 0.759 | 0.897 | 0.003 |
| | | | | alpha | | | | |
| D-dimer | IGFBP-2 | CA2D3 | PGCB | MXRA7 | 0.980 | 0.769 | 0.896 | 0.021 |
| D-dimer | IP-10 | CD79A | PGCB | JKIP3 | 0.947 | 0.788 | 0.896 | 0.015 |
| MMP-1 | IP-10 | PKB | MXRA7 | NDUB4 | 0.953 | 0.801 | 0.896 | 0.042 |
| | | a/b/g | | | | | | |
| IP-10 | PKB a/b/g | PABP3 | CD36 | NDUB4 | 0.962 | 0.751 | 0.896 | 0.026 |
| | | | ANTIGEN | | | | | |
| C9 | IGFBP-2 | MXRA7 | NDUB4 | BOC | 0.973 | 0.756 | 0.896 | 0.005 |
| C9 | D-dimer | IP-10 | PABP3 | MXRA7 | 0.973 | 0.761 | 0.896 | 0.011 |
| MMP-1 | IP-10 | IGFBP-2 | PCI | BOC | 0.942 | 0.790 | 0.896 | 0.022 |
| D-dimer | IP-10 | IGFBP-2 | CA2D3 | MXRA7 | 0.969 | 0.779 | 0.896 | 0.007 |
| MMP-1 | MXRA7 | CNTFR | CD36 | PKB beta | 0.956 | 0.770 | 0.896 | 0.041 |
| | | alpha | ANTIGEN | | | | | |
| C9 | MMP-2 | PGCB | PABP3 | MXRA7 | 0.963 | 0.755 | 0.896 | 0.032 |
| D-dimer | CD79A | PKB | NDUB4 | PCI | 0.944 | 0.787 | 0.896 | 0.027 |
| | | a/b/g | | | | | | |
| C9 | D-dimer | CA2D3 | PABP3 | MXRA7 | 0.967 | 0.775 | 0.896 | 0.006 |
| D-dimer | IP-10 | PABP3 | Nr-CAM | NDUB4 | 0.943 | 0.782 | 0.896 | 0.023 |
| MMP-1 | IP-10 | PGCB | PABP3 | CNTFR | 0.951 | 0.795 | 0.896 | 0.031 |
| | | | | alpha | | | | |
| MMP-1 | IP-10 | IGFBP-2 | MXRA7 | BOC | 0.959 | 0.769 | 0.895 | 0.021 |
| MMP-1 | IP-10 | IGFBP-2 | PABP3 | PKB beta | 0.955 | 0.768 | 0.895 | 0.033 |
| D-dimer | IP-10 | IGFBP-2 | PGCB | PABP3 | 0.971 | 0.757 | 0.895 | 0.008 |
| D-dimer | IP-10 | IGFBP-2 | MXRA7 | CD36 | 0.972 | 0.754 | 0.895 | 0.008 |
| | | | | ANTIGEN | | | | |
| C9 | D-dimer | IGFBP-2 | MXRA7 | CNTFR | 0.978 | 0.751 | 0.895 | 0.005 |
| | | | | alpha | | | | |
| C9 | IP-10 | MMP-2 | PKB a/b/g | MXRA7 | 0.966 | 0.760 | 0.895 | 0.026 |
| C9 | D-dimer | CD79A | PABP3 | MXRA7 | 0.968 | 0.758 | 0.895 | 0.016 |
| D-dimer | IP-10 | IGFBP-2 | CA2D3 | CD36 | 0.965 | 0.755 | 0.895 | 0.004 |
| | | | | ANTIGEN | | | | |
| C9 | D-dimer | IP-10 | IGFBP-2 | JKIP3 | 0.952 | 0.787 | 0.895 | 0.004 |
| C9 | D-dimer | PKB | NDUB4 | PKB beta | 0.967 | 0.750 | 0.895 | 0.007 |
| | | a/b/g | | | | | | |
| C9 | D-dimer | IP-10 | MXRA7 | CD36 | 0.971 | 0.758 | 0.895 | 0.017 |
| | | | | ANTIGEN | | | | |
| C9 | IP-10 | IGFBP-2 | PABP3 | JKIP3 | 0.971 | 0.755 | 0.895 | 0.004 |
| IP-10 | MMP-2 | PKB | PABP3 | PKB beta | 0.961 | 0.774 | 0.895 | 0.049 |
| | | a/b/g | | | | | | |
| D-dimer | IP-10 | CA2D3 | PABP3 | MXRA7 | 0.959 | 0.763 | 0.895 | 0.020 |
| IP-10 | IGFBP-2 | PGCB | PABP3 | NDUB4 | 0.954 | 0.756 | 0.895 | 0.008 |
| D-dimer | IP-10 | CD79A | PABP3 | Nr-CAM | 0.953 | 0.771 | 0.894 | 0.024 |
| C9 | IP-10 | IGFBP-2 | CD79A | PABP3 | 0.962 | 0.752 | 0.894 | 0.005 |
| MMP-1 | IP-10 | PKB | BOC | PKB beta | 0.956 | 0.767 | 0.894 | 0.037 |
| | | a/b/g | | | | | | |
| C9 | IP-10 | PABP3 | Nr-CAM | NDUB4 | 0.946 | 0.794 | 0.894 | 0.017 |
| D-dimer | IP-10 | MMP-2 | MXRA7 | CD36 | 0.967 | 0.750 | 0.894 | 0.015 |
| | | | | ANTIGEN | | | | |
| C9 | IGFBP-2 | PABP3 | Nr-CAM | PKB beta | 0.962 | 0.765 | 0.894 | 0.006 |
| D-dimer | CA2D3 | PABP3 | MXRA7 | JKIP3 | 0.945 | 0.784 | 0.894 | 0.048 |
| C9 | D-dimer | IP-10 | MXRA7 | CNTFR | 0.958 | 0.778 | 0.894 | 0.004 |
| | | | | alpha | | | | |
| C9 | IP-10 | CA2D3 | MXRA7 | JKIP3 | 0.958 | 0.776 | 0.894 | 0.012 |
| C9 | D-dimer | IGFBP-2 | PABP3 | BOC | 0.967 | 0.763 | 0.894 | 0.005 |
| C9 | IP-10 | CD79A | PABP3 | NDUB4 | 0.961 | 0.769 | 0.894 | 0.016 |
| D-dimer | IP-10 | NPS | PABP3 | Nr-CAM | 0.939 | 0.771 | 0.894 | 0.025 |
| C9 | IP-10 | IGFBP-2 | CA2D3 | MXRA7 | 0.963 | 0.758 | 0.894 | 0.007 |
| C9 | D-dimer | PABP3 | PKB beta | JKIP3 | 0.961 | 0.757 | 0.894 | 0.006 |
| | | | CD36 | | | | | |
| D-dimer | IP-10 | CA2D3 | ANTIGEN | NDUB4 | 0.975 | 0.758 | 0.894 | 0.014 |
| C9 | D-dimer | IP-10 | MMP-2 | MXRA7 | 0.966 | 0.787 | 0.894 | 0.035 |
| MMP-1 | C9 | IP-10 | MXRA7 | PKB beta | 0.976 | 0.782 | 0.894 | 0.046 |
| IP-10 | CD79A | PABP3 | MXRA7 | NDUB4 | 0.953 | 0.758 | 0.894 | 0.019 |
| D-dimer | IP-10 | IGFBP-2 | NDUB4 | PCI | 0.951 | 0.772 | 0.894 | 0.015 |
| C9 | IGFBP-2 | MMP-2 | CD79A | MXRA7 | 0.976 | 0.772 | 0.894 | 0.016 |
| C9 | PKB a/b/g | PABP3 | MXRA7 | JKIP3 | 0.968 | 0.765 | 0.894 | 0.010 |
| IP-10 | IGFBP-2 | PKB | PABP3 | NDUB4 | 0.958 | 0.754 | 0.893 | 0.017 |
| | | a/b/g | | | | | | |
| C9 | IP-10 | IGFBP-2 | MXRA7 | NDUB4 | 0.967 | 0.755 | 0.893 | 0.005 |
| D-dimer | IP-10 | PABP3 | MXRA7 | JKIP3 | 0.959 | 0.774 | 0.893 | 0.016 |
| C9 | IGFBP-2 | NDUB4 | PCI | BOC | 0.952 | 0.767 | 0.893 | 0.014 |
| C9 | IP-10 | MMP-2 | PKB a/b/g | CD36 | 0.961 | 0.787 | 0.893 | 0.019 |
| | | | | ANTIGEN | | | | |
| C9 | CD79A | PABP3 | MXRA7 | NDUB4 | 0.966 | 0.760 | 0.893 | 0.006 |
| D-dimer | CD79A | PABP3 | MXRA7 | NDUB4 | 0.955 | 0.766 | 0.893 | 0.024 |
| D-dimer | IP-10 | IGFBP-2 | PKB a/b/g | PABP3 | 0.959 | 0.761 | 0.893 | 0.029 |
| IP-10 | CA2D3 | PABP3 | MXRA7 | JKIP3 | 0.953 | 0.792 | 0.893 | 0.009 |
| IP-10 | NPS | CA2D3 | PABP3 | MXRA7 | 0.947 | 0.776 | 0.893 | 0.029 |
| D-dimer | IP-10 | CA2D3 | CD36 | PKB beta | 0.967 | 0.751 | 0.893 | 0.011 |
| | | | ANTIGEN | | | | | |
| D-dimer | CA2D3 | PGCB | MXRA7 | NDUB4 | 0.965 | 0.764 | 0.893 | 0.017 |
| C9 | D-dimer | IP-10 | PKB a/b/g | NDUB4 | 0.962 | 0.760 | 0.893 | 0.039 |
| C9 | IGFBP-2 | CD79A | PABP3 | Nr-CAM | 0.964 | 0.769 | 0.893 | 0.005 |
| D-dimer | IP-10 | IGFBP-2 | PABP3 | PCI | 0.951 | 0.769 | 0.893 | 0.012 |
| C9 | IP-10 | PABP3 | NDUB4 | JKIP3 | 0.961 | 0.758 | 0.893 | 0.007 |
| D-dimer | IGFBP-2 | CD79A | PCI | BOC | 0.950 | 0.789 | 0.893 | 0.020 |
| IP-10 | CD79A | PKB | PABP3 | NDUB4 | 0.954 | 0.791 | 0.893 | 0.018 |
| | | a/b/g | | | | | | |
| C9 | IP-10 | NPS | CA2D3 | MXRA7 | 0.960 | 0.769 | 0.893 | 0.015 |
| MMP-1 | IGFBP-2 | CD79A | MXRA7 | CNTFR | 0.949 | 0.781 | 0.893 | 0.030 |
| | | | | alpha | | | | |
| D-dimer | CA2D3 | MXRA7 | NDUB4 | JKIP3 | 0.956 | 0.786 | 0.893 | 0.028 |
| D-dimer | IP-10 | IGFBP-2 | PABP3 | Nr-CAM | 0.955 | 0.783 | 0.893 | 0.009 |
| MMP-1 | IP-10 | MXRA7 | CD36 | NDUB4 | 0.967 | 0.759 | 0.892 | 0.038 |
| | | | ANTIGEN | | | | | |
| C9 | IGFBP-2 | CD79A | MXRA7 | Nr-CAM | 0.958 | 0.756 | 0.892 | 0.010 |
| IP-10 | IGFBP-2 | PABP3 | Nr-CAM | NDUB4 | 0.953 | 0.755 | 0.892 | 0.011 |
| MMP-1 | IP-10 | PABP3 | CNTFR | JKIP3 | 0.925 | 0.817 | 0.892 | 0.042 |
| | | | alpha | | | | | |
| MMP-1 | IP-10 | PKB | MXRA7 | CNTFR | 0.936 | 0.803 | 0.892 | 0.047 |
| | | a/b/g | | alpha | | | | |
| C9 | IGFBP-2 | MMP-2 | PABP3 | PCI | 0.955 | 0.753 | 0.892 | 0.028 |
| C9 | IP-10 | NPS | MXRA7 | JKIP3 | 0.951 | 0.795 | 0.892 | 0.015 |
| D-dimer | IP-10 | CD79A | PKB a/b/g | PCI | 0.938 | 0.782 | 0.892 | 0.031 |
| C9 | D-dimer | Nr-CAM | NDUB4 | PKB beta | 0.967 | 0.751 | 0.892 | 0.011 |
| C9 | IGFBP-2 | CD79A | PABP3 | BOC | 0.962 | 0.762 | 0.892 | 0.002 |
| C9 | D-dimer | IP-10 | NPS | PABP3 | 0.958 | 0.766 | 0.892 | 0.020 |
| MMP-1 | IP-10 | IGFBP-2 | CD36 | BOC | 0.958 | 0.773 | 0.892 | 0.034 |
| | | | ANTIGEN | | | | | |
| C9 | D-dimer | IP-10 | MMP-2 | CD36 | 0.969 | 0.753 | 0.892 | 0.020 |
| | | | | ANTIGEN | | | | |
| C9 | IP-10 | CA2D3 | CD36 | JKIP3 | 0.962 | 0.750 | 0.892 | 0.003 |
| | | | ANTIGEN | | | | | |
| C9 | D-dimer | MMP-2 | CD36 | PKB beta | 0.963 | 0.759 | 0.892 | 0.016 |
| | | | ANTIGEN | | | | | |
| MMP-1 | PKB a/b/g | PABP3 | MXRA7 | PKB beta | 0.964 | 0.754 | 0.891 | 0.046 |
| C9 | D-dimer | IGFBP-2 | MMP-2 | JKIP3 | 0.964 | 0.762 | 0.891 | 0.035 |
| C9 | IGFBP-2 | PKB | PABP3 | BOC | 0.969 | 0.767 | 0.891 | 0.012 |
| | | a/b/g | | | | | | |
| C9 | D-dimer | PKB | MXRA7 | Nr-CAM | 0.954 | 0.776 | 0.891 | 0.021 |
| | | a/b/g | | | | | | |
| C9 | IP-10 | IGFBP-2 | MXRA7 | Nr-CAM | 0.956 | 0.768 | 0.891 | 0.006 |
| C9 | IP-10 | NPS | PABP3 | MXRA7 | 0.953 | 0.772 | 0.891 | 0.017 |
| D-dimer | IP-10 | IGFBP-2 | PCI | JKIP3 | 0.951 | 0.784 | 0.891 | 0.009 |
| IP-10 | IGFBP-2 | MMP-2 | PABP3 | NDUB4 | 0.959 | 0.759 | 0.891 | 0.014 |
| MMP-1 | MXRA7 | NDUB4 | BOC | PKB beta | 0.953 | 0.762 | 0.891 | 0.045 |
| C9 | IGFBP-2 | CD79A | NDUB4 | PKB beta | 0.961 | 0.754 | 0.891 | 0.006 |
| C9 | D-dimer | CA2D3 | BOC | JKIP3 | 0.968 | 0.760 | 0.891 | 0.009 |
| C9 | IP-10 | IGFBP-2 | CD79A | NDUB4 | 0.955 | 0.758 | 0.891 | 0.005 |
| D-dimer | IP-10 | MMP-2 | CNTFR | CD36 | 0.971 | 0.759 | 0.891 | 0.009 |
| | | | alpha | ANTIGEN | | | | |
| C9 | MMP-2 | CNTFR | Nr-CAM | NDUB4 | 0.960 | 0.755 | 0.891 | 0.032 |
| | | alpha | | | | | | |
| IP-10 | IGFBP-2 | CA2D3 | CD79A | PABP3 | 0.967 | 0.763 | 0.891 | 0.008 |
| C9 | D-dimer | IP-10 | MMP-2 | PABP3 | 0.954 | 0.763 | 0.891 | 0.043 |
| IP-10 | IGFBP-2 | PKB | MXRA7 | CNTFR | 0.961 | 0.754 | 0.891 | 0.010 |
| | | a/b/g | | alpha | | | | |
| MMP-1 | IP-10 | MXRA7 | NDUB4 | JKIP3 | 0.948 | 0.795 | 0.891 | 0.041 |
| C9 | MMP-2 | PABP3 | MXRA7 | JKIP3 | 0.967 | 0.768 | 0.891 | 0.029 |
| D-dimer | IP-10 | CD79A | PKB a/b/g | MXRA7 | 0.949 | 0.756 | 0.891 | 0.034 |
| MMP-1 | IP-10 | IGFBP-2 | CA2D3 | CD36 | 0.951 | 0.807 | 0.891 | 0.029 |
| | | | | ANTIGEN | | | | |
| C9 | D-dimer | NPS | MXRA7 | JKIP3 | 0.968 | 0.760 | 0.891 | 0.020 |
| MMP-1 | IP-10 | MXRA7 | CNTFR | CD36 | 0.960 | 0.760 | 0.890 | 0.021 |
| | | | alpha | ANTIGEN | | | | |
| C9 | D-dimer | MMP-2 | PABP3 | JKIP3 | 0.953 | 0.752 | 0.890 | 0.033 |
| C9 | IP-10 | MXRA7 | Nr-CAM | JKIP3 | 0.952 | 0.754 | 0.890 | 0.007 |
| C9 | IP-10 | MMP-2 | PABP3 | MXRA7 | 0.955 | 0.769 | 0.890 | 0.031 |
| C9 | PABP3 | MXRA7 | Nr-CAM | JKIP3 | 0.957 | 0.759 | 0.890 | 0.019 |
| D-dimer | IP-10 | IGFBP-2 | PABP3 | BOC | 0.961 | 0.774 | 0.890 | 0.020 |
| D-dimer | IP-10 | PKB | MXRA7 | JKIP3 | 0.957 | 0.775 | 0.890 | 0.034 |
| | | a/b/g | | | | | | |
| C9 | IGFBP-2 | MMP-2 | PABP3 | MXRA7 | 0.964 | 0.760 | 0.890 | 0.023 |
| C9 | IP-10 | PKB | PABP3 | NDUB4 | 0.955 | 0.757 | 0.890 | 0.023 |
| | | a/b/g | | | | | | |
| IP-10 | IGFBP-2 | CA2D3 | PABP3 | NDUB4 | 0.963 | 0.753 | 0.890 | 0.007 |
| C9 | IP-10 | IGFBP-2 | CA2D3 | PABP3 | 0.960 | 0.750 | 0.890 | 0.004 |
| C9 | MMP-2 | CD79A | PABP3 | NDUB4 | 0.953 | 0.781 | 0.889 | 0.018 |
| MMP-1 | IP-10 | CA2D3 | CNTFR | CD36 | 0.953 | 0.782 | 0.889 | 0.015 |
| | | | alpha | ANTIGEN | | | | |
| MMP-1 | PKB a/b/g | CD36 | BOC | PKB beta | 0.956 | 0.757 | 0.889 | 0.043 |
| | | ANTIGEN | | | | | | |
| C9 | D-dimer | IP-10 | CA2D3 | JKIP3 | 0.952 | 0.786 | 0.889 | 0.006 |
| C9 | IP-10 | MXRA7 | CD36 | JKIP3 | 0.973 | 0.770 | 0.889 | 0.009 |
| | | | ANTIGEN | | | | | |
| C9 | D-dimer | NPS | BOC | JKIP3 | 0.953 | 0.752 | 0.888 | 0.010 |
| IP-10 | IGFBP-2 | PABP3 | NDUB4 | JKIP3 | 0.957 | 0.751 | 0.888 | 0.009 |
| C9 | D-dimer | IP-10 | CD79A | JKIP3 | 0.933 | 0.766 | 0.888 | 0.010 |
| IP-10 | NPS | CA2D3 | MXRA7 | NDUB4 | 0.961 | 0.758 | 0.888 | 0.038 |
| C9 | MMP-2 | CNTFR | NDUB4 | JKIP3 | 0.969 | 0.751 | 0.888 | 0.014 |
| | | alpha | | | | | | |
| MMP-1 | IP-10 | CD36 | PCI | PKB beta | 0.939 | 0.777 | 0.888 | 0.050 |
| | | ANTIGEN | | | | | | |
| D-dimer | IP-10 | MXRA7 | NDUB4 | JKIP3 | 0.956 | 0.774 | 0.888 | 0.009 |
| C9 | D-dimer | IGFBP-2 | PABP3 | PKB beta | 0.968 | 0.750 | 0.888 | 0.007 |
| C9 | IGFBP-2 | CA2D3 | CD79A | PABP3 | 0.974 | 0.758 | 0.888 | 0.004 |
| D-dimer | MMP-2 | PKB | CD36 | PKB beta | 0.959 | 0.762 | 0.888 | 0.018 |
| | | a/b/g | ANTIGEN | | | | | |
| MMP-1 | PGCB | PABP3 | BOC | PKB beta | 0.943 | 0.779 | 0.888 | 0.038 |
| D-dimer | CA2D3 | PABP3 | PCI | JKIP3 | 0.943 | 0.806 | 0.888 | 0.033 |
| C9 | D-dimer | IP-10 | PKB a/b/g | JKIP3 | 0.951 | 0.781 | 0.888 | 0.022 |
| D-dimer | IP-10 | MXRA7 | NDUB4 | PKB beta | 0.961 | 0.752 | 0.888 | 0.038 |
| C9 | IP-10 | MMP-2 | PKB a/b/g | NDUB4 | 0.947 | 0.774 | 0.887 | 0.035 |
| D-dimer | IP-10 | IGFBP-2 | CA2D3 | CD79A | 0.961 | 0.762 | 0.887 | 0.005 |
| D-dimer | IP-10 | PABP3 | NDUB4 | JKIP3 | 0.956 | 0.771 | 0.887 | 0.012 |
| MMP-1 | IP-10 | IGFBP-2 | CNTFR | CD36 | 0.958 | 0.754 | 0.887 | 0.018 |
| | | | alpha | ANTIGEN | | | | |
| C9 | PABP3 | Nr-CAM | NDUB4 | PKB beta | 0.963 | 0.752 | 0.887 | 0.011 |
| D-dimer | IP-10 | CA2D3 | MXRA7 | PKB beta | 0.958 | 0.768 | 0.887 | 0.020 |
| MMP-1 | IGFBP-2 | MXRA7 | PCI | BOC | 0.949 | 0.786 | 0.887 | 0.042 |
| D-dimer | IP-10 | NPS | CA2D3 | MXRA7 | 0.942 | 0.766 | 0.886 | 0.024 |
| C9 | D-dimer | IP-10 | MMP-2 | JKIP3 | 0.942 | 0.776 | 0.886 | 0.040 |
| MMP-1 | IP-10 | CNTFR | CD36 | NDUB4 | 0.956 | 0.773 | 0.886 | 0.046 |
| | | alpha | ANTIGEN | | | | | |
| MMP-1 | IGFBP-2 | CA2D3 | MXRA7 | PKB beta | 0.956 | 0.784 | 0.886 | 0.043 |
| C9 | IP-10 | NPS | MXRA7 | NDUB4 | 0.953 | 0.755 | 0.886 | 0.014 |
| D-dimer | IP-10 | PABP3 | MXRA7 | NDUB4 | 0.955 | 0.762 | 0.886 | 0.031 |
| C9 | IGFBP-2 | MMP-2 | PKB a/b/g | CNTFR | 0.957 | 0.767 | 0.886 | 0.009 |
| | | | | alpha | | | | |
| C9 | D-dimer | MMP-2 | PKB beta | JKIP3 | 0.955 | 0.753 | 0.886 | 0.033 |
| C9 | D-dimer | IP-10 | NPS | MXRA7 | 0.956 | 0.750 | 0.886 | 0.031 |
| IP-10 | IGFBP-2 | CA2D3 | MXRA7 | PCI | 0.953 | 0.751 | 0.886 | 0.013 |
| C9 | IP-10 | MXRA7 | NDUB4 | BOC | 0.966 | 0.752 | 0.886 | 0.015 |
| C9 | IP-10 | PKB | MXRA7 | BOC | 0.956 | 0.759 | 0.885 | 0.011 |
| | | a/b/g | | | | | | |
| IP-10 | PABP3 | MXRA7 | NDUB4 | PCI | 0.947 | 0.765 | 0.885 | 0.021 |
| C9 | D-dimer | IGFBP-2 | MMP-2 | BOC | 0.963 | 0.770 | 0.885 | 0.033 |
| C9 | D-dimer | IP-10 | IGFBP-2 | CD79A | 0.952 | 0.770 | 0.885 | 0.005 |
| MMP-1 | IP-10 | IGFBP-2 | CNTFR | JKIP3 | 0.946 | 0.783 | 0.885 | 0.026 |
| | | | alpha | | | | | |
| D-dimer | IP-10 | CD79A | MXRA7 | JKIP3 | 0.950 | 0.764 | 0.885 | 0.008 |
| MMP-1 | IP-10 | IGFBP-2 | CNTFR | NDUB4 | 0.945 | 0.794 | 0.885 | 0.016 |
| | | | alpha | | | | | |
| C9 | D-dimer | PABP3 | Nr-CAM | PKB beta | 0.955 | 0.754 | 0.885 | 0.013 |
| C9 | D-dimer | MXRA7 | Nr-CAM | JKIP3 | 0.956 | 0.772 | 0.885 | 0.012 |
| D-dimer | IP-10 | PKB | MXRA7 | NDUB4 | 0.957 | 0.762 | 0.885 | 0.037 |
| | | a/b/g | | | | | | |
| MMP-1 | IP-10 | IGFBP-2 | CNTFR | PKB beta | 0.946 | 0.788 | 0.885 | 0.024 |
| | | | alpha | | | | | |
| D-dimer | IP-10 | IGFBP-2 | CNTFR | PCI | 0.946 | 0.755 | 0.885 | 0.008 |
| | | | alpha | | | | | |
| C9 | IP-10 | CA2D3 | PABP3 | PKB beta | 0.962 | 0.755 | 0.884 | 0.012 |
| D-dimer | IP-10 | IGFBP-2 | PKB a/b/g | PGCB | 0.956 | 0.752 | 0.884 | 0.013 |
| MMP-1 | IP-10 | IGFBP-2 | PKB a/b/g | PKB beta | 0.954 | 0.783 | 0.884 | 0.017 |
| D-dimer | MMP-2 | MXRA7 | CD36 | PCI | 0.963 | 0.768 | 0.884 | 0.042 |
| | | | ANTIGEN | | | | | |
| IP-10 | CA2D3 | MXRA7 | NDUB4 | JKIP3 | 0.958 | 0.752 | 0.884 | 0.011 |
| IP-10 | PABP3 | MXRA7 | NDUB4 | JKIP3 | 0.940 | 0.782 | 0.884 | 0.015 |
| D-dimer | IGFBP-2 | CA2D3 | CD79A | PCI | 0.949 | 0.783 | 0.884 | 0.017 |
| D-dimer | IP-10 | PGCB | PABP3 | JKIP3 | 0.947 | 0.766 | 0.884 | 0.021 |
| C9 | IP-10 | PKB | MXRA7 | Nr-CAM | 0.947 | 0.754 | 0.883 | 0.011 |
| | | a/b/g | | | | | | |
| IP-10 | IGFBP-2 | PABP3 | MXRA7 | NDUB4 | 0.953 | 0.775 | 0.883 | 0.018 |
| C9 | IP-10 | MMP-2 | MXRA7 | PKB beta | 0.958 | 0.750 | 0.883 | 0.014 |
| C9 | D-dimer | IGFBP-2 | MMP-2 | PKB | 0.961 | 0.756 | 0.883 | 0.028 |
| | | | | a/b/g | | | | |
| C9 | CA2D3 | MMP-2 | PKB beta | JKIP3 | 0.953 | 0.751 | 0.882 | 0.031 |
| MMP-1 | IGFBP-2 | MXRA7 | NDUB4 | BOC | 0.946 | 0.757 | 0.882 | 0.024 |
| D-dimer | IP-10 | CA2D3 | MXRA7 | PCI | 0.946 | 0.780 | 0.880 | 0.024 |
| IP-10 | CA2D3 | PKB | PABP3 | MXRA7 | 0.946 | 0.785 | 0.879 | 0.031 |
| | | a/b/g | | | | | | |
| C9 | IGFBP-2 | CA2D3 | PKB a/b/g | MXRA7 | 0.967 | 0.768 | 0.877 | 0.005 |
| MMP-1 | IGFBP-2 | MXRA7 | CNTFR | PKB beta | 0.947 | 0.761 | 0.877 | 0.040 |
| | | | alpha | | | | | |
| D-dimer | IP-10 | NPS | PABP3 | NDUB4 | 0.943 | 0.777 | 0.876 | 0.022 |

TABLE C
5 Protein Models with AUC for 0-6 months of >0.8

| Prot1 | Prot2 | Prot3 | Prot4 | Prot5 | AUC_0_180 | AUC_180_360 | AUC_Weight | JTpval |
|---|---|---|---|---|---|---|---|---|
| MMP-1 | IP-10 | IGFBP-2 | PABP3 | MXRA7 | 0.960 | 0.812 | 0.917 | 0.022 |
| C9 | D-dimer | MMP-2 | PABP3 | MXRA7 | 0.971 | 0.803 | 0.913 | 0.034 |
| MMP-1 | IP-10 | IGFBP-2 | MXRA7 | NDUB4 | 0.960 | 0.821 | 0.913 | 0.023 |
| MMP-1 | IP-10 | PABP3 | MXRA7 | NDUB4 | 0.964 | 0.824 | 0.909 | 0.023 |
| MMP-1 | IP-10 | IGFBP-2 | CA2D3 | MXRA7 | 0.959 | 0.823 | 0.909 | 0.027 |
| MMP-1 | IP-10 | PKB | PABP3 | MXRA7 | 0.945 | 0.823 | 0.907 | 0.046 |
| | | a/b/g | | | | | | |
| MMP-1 | IP-10 | CA2D3 | PABP3 | MXRA7 | 0.955 | 0.822 | 0.907 | 0.032 |
| MMP-1 | IP-10 | IGFBP-2 | PABP3 | CD36 | 0.967 | 0.801 | 0.907 | 0.029 |
| | | | | ANTIGEN | | | | |
| MMP-1 | IP-10 | IGFBP-2 | MXRA7 | CNTFR | 0.959 | 0.819 | 0.906 | 0.017 |
| | | | | alpha | | | | |
| C9 | IP-10 | CA2D3 | PKB | MXRA7 | 0.960 | 0.812 | 0.906 | 0.009 |
| | | | a/b/g | | | | | |
| MMP-1 | IP-10 | PABP3 | MXRA7 | PCI | 0.950 | 0.817 | 0.904 | 0.042 |
| MMP-1 | IP-10 | PABP3 | MXRA7 | CNTFR | 0.961 | 0.819 | 0.904 | 0.038 |
| | | | | alpha | | | | |
| MMP-1 | IP-10 | IGFBP-2 | PKB | MXRA7 | 0.956 | 0.804 | 0.904 | 0.037 |
| | | | a/b/g | | | | | |
| MMP-1 | IP-10 | IGFBP-2 | PABP3 | PCI | 0.947 | 0.805 | 0.903 | 0.040 |
| MMP-1 | IP-10 | IGFBP-2 | MXRA7 | PCI | 0.953 | 0.813 | 0.903 | 0.030 |
| MMP-1 | IP-10 | CA2D3 | MXRA7 | NDUB4 | 0.956 | 0.811 | 0.903 | 0.050 |
| MMP-1 | IP-10 | IGFBP-2 | PABP3 | NDUB4 | 0.959 | 0.802 | 0.903 | 0.044 |
| MMP-1 | IP-10 | PGCB | MXRA7 | CNTFR | 0.954 | 0.815 | 0.902 | 0.021 |
| | | | | alpha | | | | |
| IP-10 | IGFBP-2 | PABP3 | NDUB4 | PCI | 0.948 | 0.802 | 0.901 | 0.007 |
| MMP-1 | IP-10 | PABP3 | MXRA7 | JKIP3 | 0.953 | 0.821 | 0.900 | 0.046 |
| MMP-1 | IP-10 | CD79A | PABP3 | MXRA7 | 0.942 | 0.815 | 0.900 | 0.038 |
| C9 | D-dimer | IP-10 | MXRA7 | Nr-CAM | 0.959 | 0.803 | 0.900 | 0.012 |
| MMP-1 | IP-10 | PABP3 | CNTFR | NDUB4 | 0.949 | 0.807 | 0.897 | 0.036 |
| | | | alpha | | | | | |
| MMP-1 | IP-10 | MXRA7 | CNTFR | NDUB4 | 0.950 | 0.822 | 0.897 | 0.039 |
| | | | alpha | | | | | |
| MMP-1 | IP-10 | PKB | MXRA7 | NDUB4 | 0.953 | 0.801 | 0.896 | 0.042 |
| | | a/b/g | | | | | | |
| MMP-1 | IP-10 | PABP3 | CNTFR | JKIP3 | 0.925 | 0.817 | 0.892 | 0.042 |
| | | | alpha | | | | | |
| MMP-1 | IP-10 | PKB | MXRA7 | CNTFR | 0.936 | 0.803 | 0.892 | 0.047 |
| | | a/b/g | | alpha | | | | |
| MMP-1 | IP-10 | IGFBP-2 | CA2D3 | CD36 | 0.951 | 0.807 | 0.891 | 0.029 |
| | | | | ANTIGEN | | | | |
| D-dimer | CA2D3 | PABP3 | PCI | JKIP3 | 0.943 | 0.806 | 0.888 | 0.033 |

1-36. (canceled)

37. A kit comprising at least two, at least three, at least four, or at least five protein biomarker capture reagents, wherein each protein biomarker capture reagent specifically binds to a target selected from C9, IGFBP-2, CD79A, MXRA7, and Nr-CAM, and wherein each protein biomarker capture reagent specifically binds to a different target protein.

38-43. (canceled)

44. A composition comprising proteins of a sample from a subject and at least two, at least three, at least four, or at least five protein biomarker capture reagents, wherein each protein biomarker capture reagent specifically binds to a target protein selected from C9, IGFBP-2, CD79A, MXRA7, and Nr-CAM, and wherein each protein biomarker capture reagent specifically binds a different target protein.

45-56. (canceled)

57. The kit of claim 37, further comprising one or more of: a solid support, a signal generating material, instructions for use of the kit, a computer system or software to analyze the data, one or more reagents for processing a biological sample from a subject, blocking agents, positive control samples, and negative control samples.

58. The kit of claim 37, wherein at least one biomarker capture reagent includes an antibody or aptamer.

59. The kit of claim 57 wherein the instructions for use of the kit include instructions for monitoring a latent TB infection in a subject for the likelihood of the latent TB infection transitioning to active TB disease comprising detecting the level of at least two, at least three, at least four or at least five of the target proteins in a biological sample from a subject, wherein the subject is identified as having a latent TB infection that is likely to transition into active TB disease if the level of at least two, at least three, at least four or at least five of the target proteins is altered relative to a control level of the respective target proteins.

60. The kit of claim 59, wherein the one or more target proteins are indicative of one or more of: the presence of latent TB infection, the presence of active TB disease, the strain of TB, the antibiotic resistance/sensitivity of TB, and/or the presence of other diseases.

61. The kit of claim 59, wherein the subject is identified as being likely to transition to active TB disease within 30 days, 45 days, 60 days, 90 days, 120 days, 180 days, 270 days, 360 days, 450 days, or 540 days if the level of the target protein(s) detected is altered relative to a control level of the target protein(s).

62. The kit of claim 59, wherein the monitoring a latent TB infection in a subject for the likelihood of the latent TB infection transitioning to active TB disease comprises detecting the level of at least at least two, at least three, at least four or at least five of the target proteins in a sample from a subject at a first time point, and measuring the level of the respective target proteins in a sample from the subject at a second time point, wherein:
(i) if the level of at least two, at least three, at least four or at least five of the target proteins is further from a control level of the respective target proteins at the second time point than at the first time point, the likelihood of the latent TB infection transitioning to active TB disease has increased; and
(ii) if the level of at least two, at least three, at least four or at least five of the target proteins is closer to a control level of the respective target proteins at the second time point than at the first time point, the likelihood of the latent TB infection transitioning to active TB disease has decreased.

63. The kit of claim 62, wherein:
(i) a level of one or both of target proteins C9 and IGFBP-2 that is higher at the second time point than the first time point, and/or a level of one or more of target proteins CD79A, MXRA7 and Nr-CAM that is lower at the second time point than at the first time point, indicates that the likelihood of the latent TB infection transitioning to active TB disease has increased; and
(ii) a level of one or more of target proteins C9 and IGFBP-2 that is lower at the second time point than the first time point, and/or a level of one or more of target proteins CD79A, MXRA7 and Nr-CAM that is higher at the second time point than at the first time point, indicates that the likelihood of the latent TB infection transitioning to active TB disease has decreased.

64. The kit of claim 57, wherein the instructions for use of the kit include instructions for monitoring treatment of a latent TB infection, comprising detecting the level of at least at least two, at least three, at least four or at least five of the target proteins in a biological sample from a subject at a first time point, administering at least one treatment for TB infection to the subject, and detecting the level of the respective target proteins in a sample from the subject at a second time point, wherein the treatment is effective at reducing the likelihood of the latent TB infection transitioning to active TB disease if the level of the target proteins is closer to a control level of the respective target proteins at the second time point compared to the first time point.

65. The kit of claim 64, wherein a level of one or both of target proteins C9 and IGFBP-2 that is lower at the second time point than the first time point, and/or a level of one or more of target proteins CD79A, MXRA7 and Nr-CAM that is higher at the second time point than at the first time point, indicates that the treatment is effective at reducing the likelihood of the latent TB infection transitioning to active TB disease.

66. The kit of claim 57 wherein the instructions for use direct the use of any one or more of histological or cytological detection methods, mass spectroscopy and immunoassay.

67. The composition of claim 44, wherein at least one biomarker capture reagent includes an antibody or aptamer.

68. The composition of claim 44, wherein the sample is a blood sample, a plasma sample or a serum sample.
